(19) United States
(12) Patent Application Publication
(10) Pub. No.: US 2007/0087360 A1
(43) Pub. Date: Apr. 19, 2007

(54) Methods and compositions for detecting nucleotides

(76) Inventor: Victoria L. Boyd, San Carlos, CA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/471,462

(22) Filed: Jun. 19, 2006

Related U.S. Application Data
(60) Provisional application No. 60/692,820, filed on Jun. 20, 2005.

(51) Int. Cl. C12Q 1/68

(52) U.S. Cl. 435/6

(57) ABSTRACT

The present teachings generally relate to methods and materials for the detection of target nucleotides and/or the methylation state of target nucleotides.

This application claims the benefit of U.S. Provisional Application No. 60/692,820, filed Jun. 20, 2005, which is incorporated by reference herein for any purpose.

### FIELD

The present teachings generally relate to methods and materials for the detection of target nucleotides and/or the methylation state of target nucleotides.

### BACKGROUND

Detection of target nucleotides and assessment of methylation of DNA are useful in many research, diagnostic, medical, forensic and industrial fields.

In certain instances, methylation has regulatory effects on gene expression and may play an important role in a variety of settings, including gene inactivation, cell differentiation, tumor growth, X-chromosome inactivation, and genomic imprinting. For example, in certain instances, extensive methylation in a promoter region has been shown to suppress transcription. Thus, in certain instances, methylation may play a role in developmental gene regulation and cell differentiation.

Also, aberrant methylation has been described in several tumors and immortalized and transformed cells. Hypermethylation of tumor suppressor regions has been associated with human cancers. Thus, in certain instances, determination of methylation may be useful in tumor assessment.

In certain instances, determining methylation may be used to study gene regulation and may serve as a marker for various disease states and may be useful for tissue typing. Determining methylation in certain instances may be useful for identifying individuals (i.e. fingerprinting) or for other industrial applications.

### SUMMARY

In certain embodiments, a method for determining the methylation state of a target nucleotide in at least one target nucleic acid sequence in a sample is provided. In certain embodiments, a ligation reaction composition is formed comprising the sample, at least one blocking probe, and a ligation probe set for each target nucleic acid sequence. In certain embodiments, the ligation probe set comprises (a) a first probe, comprising a first target-specific portion; and (b) a second probe, comprising a second target-specific portion. In certain embodiments, the ligation reaction composition is subjected to at least one cycle of ligation, under conditions effective to ligate together first and second probes that are hybridized adjacent to one another on the target nucleic acid sequence if the target nucleotide is methylated to form a ligation product. In certain embodiments, the blocking probe hybridizes to a portion of the target nucleic acid sequence comprising the target nucleotide if the target nucleotide is unmethylated. In certain embodiments, hybridization of the blocking probe to the portion of the target nucleic acid sequence blocks hybridization of the first probe, the second probe, or both the first probe and the second probe to the portion of the target nucleic acid sequence. In certain embodiments, the presence or absence of the ligation product is detected to determine the methylation state of the target nucleotide.

In certain embodiments, a method for determining the methylation state of a target nucleotide in at least one target nucleic acid sequence in a sample is provided. In certain embodiments, a ligation reaction composition is formed comprising the sample, at least one blocking probe, and a ligation probe set for each target nucleic acid sequence. In certain embodiments, the ligation probe set comprises (a) a first probe, comprising a first target-specific portion; and (b) a second probe, comprising a second target-specific portion. In certain embodiments, the ligation reaction composition is subjected to at least one cycle of ligation, under conditions effective to ligate together first and second probes that are hybridized adjacent to one another on the target nucleic acid sequence if the target nucleotide is unmethylated to form a ligation product. In certain embodiments, the blocking probe hybridizes to a portion of the target nucleic acid sequence comprising the target nucleotide if the target nucleotide is methylated. In certain embodiments, hybridization of the blocking probe to the portion of the target nucleic acid sequence blocks hybridization of the first probe, the second probe, or both the first probe and the second probe to the portion of the target nucleic acid sequence. In certain embodiments, the presence or absence of the ligation product is detected to determine the methylation state of the target nucleotide.

In certain embodiments, a kit for determining the methylation state of a target nucleotide in at least one target nucleic acid sequence in a sample is provided. In certain embodiments, the kit for determining the methylation state of a target nucleotide in at least one target nucleic acid sequence in a sample comprises at least one blocking probe and a ligation probe set for each target nucleic acid sequence. In certain embodiments the ligation probe set comprises: (a) a first probe, comprising a first target-specific portion, and (b) a second probe, comprising a second target-specific portion. In certain embodiments, the first probe and the second probe in each ligation probe set are suitable for ligation together when hybridized adjacent to one another on a complementary target nucleic acid sequence. In certain embodiments, the blocking probe is capable of hybridizing to a portion of the target nucleic acid sequence comprising the target nucleotide if the target nucleotide is unmethylated. In certain embodiments, the blocking probe is capable of hybridizing to a portion of the target nucleic acid sequence comprising the target nucleotide if the target nucleotide is methylated. In certain embodiments, hybridization of the blocking probe to the portion of the target nucleic acid sequence blocks hybridization of the first probe, the second probe, or both the first probe and the second probe to the portion of the target nucleic acid sequence.

In certain embodiments, a method for determining the methylation state of a target nucleotide in at least one target nucleic acid sequence in a sample is provided. In certain embodiments, a test composition is formed by incubating the at least one target nucleic acid sequence with a modifying agent that modifies unmethylated target nucleotide to a modified target nucleotide, but does not modify methylated target nucleotide to the modified target nucleotide, to obtain at least one test target nucleic acid sequence. In certain embodiments, a ligation reaction composition is formed comprising at least a portion of the test composition, at least one blocking probe, and a ligation probe set for each target nucleic acid sequence. In certain embodiments, the ligation probe set comprises (a) a first probe, comprising a first target-specific portion; and (b) a second probe, comprising a second target-specific portion. In certain embodiments, the first probe and the second probe in each ligation probe set are suitable for ligation together when hybridized adjacent to one another on a complementary test target nucleic acid sequence. In certain embodiments, at least one of the first probe and the second probe of each ligation probe set comprises a test nucleotide that aligns opposite the target nucleotide if the probe is hybridized to the test target nucleic acid sequence, wherein the test nucleotide is complementary to the target nucleotide. In certain embodiments, the blocking probe hybridizes to a portion of the test target nucleic acid sequence comprising the modified target nucleotide if the target nucleotide has been modified to the modified target nucleotide. In certain embodiments, hybridization of the blocking probe to the portion of the test target nucleic acid sequence blocks hybridization of the first probe, the second probe, or both the first probe and the second probe to the portion of the test target nucleic-acid sequence. In certain embodiments, the ligation reaction composition is subjected to at least one cycle of ligation, wherein adjacently hybridizing probes are ligated together to form a ligation product. In certain embodiments, the presence or absence of the ligation product is detected to determine the methylation state of the target nucleotide.

In certain embodiments, a method for determining the methylation state of a target nucleotide in at least one target nucleic acid sequence in a sample is provided. In certain embodiments, a test composition is formed by incubating the at least one target nucleic acid sequence with a modifying agent that modifies unmethylated target nucleotide to a modified target nucleotide, but does not modify methylated target nucleotide to the modified target nucleotide, to obtain at least one test target nucleic acid sequence. In certain embodiments, a ligation reaction composition is formed comprising at least a portion of the test composition, at least one blocking probe, and a ligation probe set for each target nucleic acid sequence. In certain embodiments, the ligation probe set comprises: (a) a first probe, comprising a first target-specific portion; and (b) a second probe, comprising a second target-specific portion. In certain embodiments, the first probe and the second probe in each ligation probe set are suitable for ligation together when hybridized adjacent to one another on a complementary test target nucleic acid sequence. In certain embodiments, at least one of the first probe and the second probe of each ligation probe set comprises a test nucleotide that aligns opposite the modified target nucleotide if the probe is hybridized to the test target nucleic acid sequence, wherein the test nucleotide is complementary to the modified target nucleotide. In certain embodiments, the blocking probe hybridizes to a portion of the test target nucleic acid sequence comprising the target nucleotide if the target nucleotide has not been modified to the modified target nucleotide. In certain embodiments, hybridization of the blocking probe to the portion of the test target nucleic acid sequence blocks hybridization of the first probe, the second probe, or both the first probe and the second probe to the portion of the test target nucleic acid sequence. In certain embodiments, the ligation reaction composition is subjected to at least one cycle of ligation, wherein adjacently hybridizing probes are ligated together to form a ligation product. In certain embodiments, the presence or absence of the ligation product is detected to determine the methylation state of the target nucleotide.

In certain embodiments, a method for determining the methylation state of a target nucleotide in at least one target nucleic acid sequence in a sample is provided. In certain embodiments, a test composition is formed by incubating the at least one target nucleic acid sequence with a modifying agent that modifies methylated target nucleotide to a modified target nucleotide, but does not modify unmethylated target nucleotide to the modified target nucleotide, to obtain at least one test target nucleic acid sequence. In certain embodiments, a ligation reaction composition, is formed comprising at least a portion of the test composition, at least one blocking probe, and a ligation probe set for each target nucleic acid sequence. In certain embodiments, the ligation probe set comprises (a) a first probe, comprising a first target-specific portion; and (b) a second probe, comprising a second target-specific portion. In certain embodiments, the first probe and the second probe in each ligation probe set are suitable for ligation together when hybridized adjacent to one another on a complementary test target nucleic acid sequence. In certain embodiments, at least one of the first probe and the second probe of each ligation probe set comprises a test nucleotide that aligns opposite the target nucleotide if the probe is hybridized to the test target nucleic acid sequence, wherein the test nucleotide is complementary to the target nucleotide. In certain embodiments, the blocking probe hybridizes to a portion of the test target nucleic acid sequence comprising the modified target nucleotide if the target nucleotide has been modified to the modified target nucleotide. In certain embodiments, hybridization of the blocking probe to the portion of the test target nucleic acid sequence blocks hybridization of the first probe, the second probe, or both the first probe and the second probe to the portion of the test target nucleic acid sequence. In certain embodiments, the ligation reaction composition is subjected to at least one cycle of ligation, wherein adjacently hybridizing probes are ligated together to form a ligation product. In certain embodiments, the presence or absence of the ligation product is detected to determine the methylation state of the target nucleotide.

In certain embodiments, method for determining the methylation state of a target nucleotide in at least one target nucleic acid sequence in a sample is provided. In certain embodiments, a test composition is formed by incubating the at least one target nucleic acid sequence with a modifying agent that modifies methylated target nucleotide to a modified target nucleotide, but does not modify unmethylated target nucleotide to the modified target nucleotide, to obtain at least one test target nucleic acid sequence. In certain embodiments, a ligation reaction composition is formed comprising at least a portion of the test composition, at least one blocking probe, and a ligation probe set for each target nucleic acid sequence. In certain embodiments, the ligation probe set comprises: (a) a first probe, comprising a first target-specific portion; and (b) a second probe, comprising a second target-specific portion. In certain embodiments, the first probe and the second probe in each ligation probe set are suitable for ligation together when hybridized adjacent to one another on a complementary test target nucleic acid sequence. In certain embodiments, at least one of the first probe and the second probe of each ligation probe set comprises a test nucleotide that aligns opposite the modified target nucleotide if the probe is hybridized to the test target nucleic acid sequence, wherein the test nucleotide is complementary to the modified target nucleotide. In certain embodiments, the blocking probe hybridizes to a portion of the test target nucleic acid sequence comprising the target nucleotide if the target nucleotide has not been modified to the modified target nucleotide. In certain embodiments, hybridization of the blocking probe to the portion of the test target nucleic acid sequence blocks hybridization of the first probe, the second probe, or both the first probe and the second probe to the portion of the test target nucleic acid sequence. In certain embodiments, the ligation reaction composition is subjected to at least one cycle of ligation, wherein adjacently hybridizing probes are ligated together to form a ligation product. In certain embodiments, the presence or absence of the ligation product is detected to determine the methylation state of the target nucleotide.

In certain embodiments, a kit for determining the methylation state of a target nucleotide in at least one target nucleic acid sequence in a sample is provided. In certain embodiments, the kit for determining the methylation state of a target nucleotide in at least one target nucleic acid sequence in a sample comprises (a) a modifying agent that modifies unmethylated target nucleotide, but does not modify methylated target nucleotide, to form a test target nucleic acid sequence; (b) at least one blocking probe; and (c) a ligation probe set for each target nucleic acid sequence. In certain embodiments, the ligation probe set comprises: (a) a first probe, comprising a first target-specific portion, and (b) a second probe, comprising a second target-specific portion. In certain embodiments, the first probe and the second probe in each ligation probe set are suitable for ligation together when hybridized adjacent to one another on a complementary target nucleic acid sequence. In certain embodiments, the blocking probe is capable of hybridizing to a portion of the test target nucleic acid sequence comprising the modified target nucleotide if the target nucleotide has been modified to the modified target nucleotide. In certain other embodiments, the blocking probe is capable of hybridizing to a portion of the test target nucleic acid sequence comprising the target nucleotide if the target nucleotide has not been modified to the modified target nucleotide. In certain embodiments, hybridization of the blocking probe to the portion of the test target nucleic acid sequence blocks hybridization of the first probe, the second probe, or both the first probe and the second probe to the portion of the test target nucleic acid sequence.

In certain embodiments, a kit for determining the methylation state of a target nucleotide in at least one target nucleic acid sequence in a sample is provided. In certain embodiments, the kit for determining the methylation state of a target nucleotide in at least one target nucleic acid sequence in a sample comprises: (a) a modifying agent that modifies methylated target nucleotide, but does not modify unmethylated target nucleotide, to form a test target nucleic acid sequence; (b) at least one blocking probe; and (c) a ligation probe set for each target nucleic acid sequence. In certain embodiments, the ligation probe set comprises: (a) a first probe, comprising a first target-specific portion, and (b) a second probe, comprising a second target-specific portion. In certain embodiments, the first probe and the second probe in each ligation probe set are suitable for ligation together when hybridized adjacent to one another on a complementary target nucleic acid sequence. In certain embodiments, the blocking probe is capable of hybridizing to a portion of the test target nucleic acid sequence comprising the modified target nucleotide if the target nucleotide has been modified to the modified target nucleotide. In certain other embodiments, the blocking probe is capable of hybridizing to a portion of the test target nucleic acid sequence comprising the target nucleotide if the target nucleotide has not been modified to the modified target nucleotide. In certain embodiments, hybridization of the blocking probe to the portion of the test target nucleic acid sequence blocks hybridization of the first probe, the second probe, or both the first probe and the second probe to the portion of the test target nucleic acid sequence.

In certain embodiments, a method for detecting a first nucleotide at a test position in at least one first target nucleic acid sequence in a sample, wherein the sample comprises at least one second target nucleic acid sequence comprising a second different nucleotide at the test position is provided. In certain embodiments, a ligation reaction composition is formed comprising: (i) the sample; (ii) at least one blocking probe, comprising at least one modification that either: (a) increases the affinity of the blocking probe for a nucleic acid sequence that is complementary to the blocking probe sequence without any mismatches, (b) decreases the affinity of the blocking probe for a nucleic acid sequence that differs by at least one nucleotide from a sequence that is complementary to the blocking probe without any mismatches, or (c) both increases the affinity of the blocking probe for a nucleic acid sequence that is complementary to the blocking probe sequence without any mismatches and decreases the affinity of the blocking probe for a nucleic acid sequence that differs by at least one nucleotide from a sequence that is complementary to the blocking probe without any mismatches; and (iii) a ligation probe set for the at least one first target nucleic acid sequence. In certain embodiments, the ligation probe set comprises: (a) a first probe, comprising a first target-specific portion; and (b) a second probe, comprising a second target-specific portion. In certain embodiments, the ligation reaction composition is subjected to at least one cycle of ligation, under conditions effective to ligate together first and second probes that are hybridized adjacent to one another on the first target nucleic acid sequence if the first nucleotide is present at the test position to form a ligation product. In certain embodiments, the blocking probe hybridizes to a portion of the second target nucleic acid sequence comprising the second different nucleotide at the test position. In certain embodiments, hybridization of the blocking probe to the portion of the second target nucleic acid sequence blocks hybridization of the first probe, the second probe, or the first probe and the second probe to the portion of the second target nucleic acid sequence. In certain embodiments, the presence or absence of the ligation product is detected to detect the first nucleotide at the test position in the at least one first target nucleic acid sequence.

In certain embodiments, a kit for detecting a first nucleotide at a test position in at least one first target nucleic acid sequence in a sample, wherein the sample comprises at least one second target nucleic acid sequence comprising a second different nucleotide at the test position, is provided. In certain embodiments the kit for detecting a first nucleotide at a test position in at least one first target nucleic acid sequence in a sample, wherein the sample comprises at least one second target nucleic acid sequence comprising a second different nucleotide at the test position, comprises: (i) at least one blocking probe, comprising at least one modification that either: (a) increases the affinity of the blocking probe for a nucleic acid sequence that is exactly complementary to the blocking probe sequence without any mismatches, (b) decreases the affinity of the blocking probe for a nucleic acid sequence that differs by at least one nucleotide from a sequence that is complementary to the blocking probe sequence without any mismatches, or (c) both increases the affinity of the blocking probe for a nucleic acid sequence that is exactly complementary to the blocking probe sequence without any mismatches and decreases the affinity of the blocking probe for a nucleic acid sequence that differs by at least one nucleotide from a sequence that is complementary to the blocking probe without any mismatches; and (ii) a ligation probe set for the first target nucleic acid sequence. In certain embodiments, the ligation probe set comprises: (a) a first probe, comprising a first target-specific portion, and (b) a second probe, comprising a second target-specific portion. In certain embodiments, the first probe and the second probe in each ligation probe set are suitable for ligation together when hybridized adjacent to one another on the first target nucleic acid sequence. In certain embodiments, the blocking probe hybridizes to a portion of the second target nucleic acid sequence comprising the second different nucleotide at the test position. In certain embodiments, hybridization of the blocking probe to the portion of the second target nucleic acid sequence blocks hybridization of the first probe, the second probe, or the first probe and the second probe to the portion of the second target nucleic acid sequence.

These and other features of the present teachings are set forth herein.

### DRAWINGS

The skilled artisan will understand that the drawings, described below, are for illustration purposes only. The figures are not intended to limit the scope of the present teachings in any way.

FIGS. 1(A) to 1(D). Schematic showing of ligation probe sets according to certain exemplary embodiments.

FIGS. 2(A) to 2(D) depict certain embodiments comprising ligation. Nucleotides marked Gm represent modified guanine. Nucleotides marked Cₘ represent methylated cytosines.

FIGS. 3(A) to 3(E) depict certain embodiments comprising amplification. Nucleotides marked Cₘrepresent methylated cytosines.

FIGS. 4(A) to 4(D) depict certain embodiments comprising ligation.

FIGS. 5(A) to 5(K) depict certain embodiments comprising ligation and amplification. Nucleotides marked Cₘrepresent methylated cytosines.

FIG. 6 depicts reaction products from three oligonucleotide reaction compositions. The three reaction compositions comprise Primers P15 Me P B-OLA (CGACGCTAACCAAACCC (SEQ ID NO.: X)); P15 Me FAM B-OLA ((FAM)-CTAATCCCCGCGCCG (SEQ ID NO.: X)); and 0, 0.5 μM, or 5 μM of P15 Blocking Probe (CCCACACCACAACACTAACC (SEQ ID NO.: X)), as described in Example 1.

FIG. 7 depicts reaction products from three oligonucleotide reaction compositions. The three reaction compositions comprise P15 UnMe P (CAACACTAACCAAACCC (SEQ ID NO.: X)); P15 UnMe ASO ((FAM)-CTAATCCCCACACCA (SEQ ID NO.: X)); and 0, 0.5 μM, or 5 μM of P15 Blocking Probe (CCCACACCACAACACTAACC (SEQ ID NO.: X)), as described in Example 1.

### DESCRIPTION OF VARIOUS EMBODIMENTS

In this application, the use of the singular includes the plural unless specifically stated otherwise. In this application, the word “a” or “an” means “at least one” unless specifically stated otherwise. In this application, the use of “or” means “and/or” unless stated otherwise. In this application, the meaning of the phrase “at least one” is equivalent to the meaning of the phrase “one or more.” Furthermore, the use of the term “including,” as well as other forms, such as “includes” and “included,” is not limiting. Also, terms such as “element” or “component” encompass both elements or components comprising one unit and elements or components that comprise more than one unit unless specifically stated otherwise.

The section headings used herein are for organizational purposes only and are not to be construed as limiting the subject matter described. All documents, or portions of documents, cited in this application, including but not limited to patents, patent applications, articles, books, and treatises, are hereby expressly incorporated by reference in their entirety for any purpose. In the event that one or more of the incorporated documents or portions of documents defines a term that contradicts that term's definition in this application, this application controls.

U.S. patent application Ser. No. 09/584,905, filed May 30, 2000, and 09/724,755, filed Nov. 28, 2000, Ser. No. 10/011,993, filed Dec. 5, 2001; U.S. Provisional Patent Application Nos. 60/412,225 filed Sep. 19, 2002, and 60/421,035 filed Oct. 23, 2002; and Patent Cooperation Treaty Application No. PCT/US01/17329, filed May 30, 2001, which published as International Publication Number WO 01/092579, are hereby expressly incorporated by reference in their entirety for any purpose.

Definitions

The term “nucleotide base” refers to a substituted or unsubstituted aromatic ring or rings. In certain embodiments, the aromatic ring or rings contain at least one nitrogen atom. In certain embodiments, the nucleotide base is capable of forming Watson-Crick and/or Hoogsteen hydrogen bonds with an appropriately complementary nucleotide base. Exemplary nucleotide bases and analogs thereof include, but are not limited to, naturally occurring nucleotide bases, e.g., adenine, guanine, cytosine, uracil, and thymine, and analogs of the naturally occurring nucleotide bases, e.g., 7-deazaadenine, 7-deazaguanine, 7-deaza-8-azaguanine, 7-deaza-8-azaadenine, N6-Δ2-isopentenyladenine (6iA), N6-Δ2-isopentenyl-2-methylthioadenine (2 ms6iA), N2-dimethylguanine (dmG), 7-methylguanine (7mG), inosine, nebularine, 2-aminopurine, 2-amino-6-chloropurine, 2,6-diaminopurine, hypoxanthine, pseudouridine, pseudocytosine, pseudoisocytosine, 5-propynylcytosine, isocytosine, isoguanine, 7-deazaguanine, 2-thiopyrimidine, 6-thioguanine, 4-thiothymine, 4-thiouracil, O⁶-methylguanine, N⁶-methyladenine, O⁴-methylthymine, 5,6-dihydrothymine, 5,6-dihydrouracil, pyrazolo[3,4-D]pyrimidines (see, e.g., U.S. Pat. Nos. 6,143,877 and 6,127,121 and PCT published application WO 01/38584), ethenoadenine, indoles such as nitroindole and 4-methylindole, and pyrroles such as nitropyrrole. Certain exemplary nucleotide bases can be found, e.g., in Fasman, 1989, Practical Handbook of Biochemistry and Molecular Biology, pp. 385-394, CRC Press, Boca Raton, Fla., and the references cited therein.

The term “nucleotide” refers to a compound comprising a nucleotide base linked to the C-1′ carbon of a sugar, such as ribose, arabinose, xylose, and pyranose, and sugar analogs thereof. The term nucleotide also encompasses nucleotide analogs. The sugar may be substituted or unsubstituted. Substituted ribose sugars include, but are not limited to, those riboses in which one or more of the carbon atoms, for example the 2′-carbon atom, is substituted with one or more of the same or different Cl, F, —R, —OR, —NR₂ or halogen groups, where each R is independently H, C₁-C₆ alkyl or C₅-C₁₄ aryl. Exemplary riboses include, but are not limited to, 2′-(C1-C6)alkoxyribose, 2′-(C5-C14)aryloxyribose, 2′,3′-didehydroribose, 2′-deoxy-3′-haloribose, 2′-deoxy-3′-fluororibose, 2′-deoxy-3′-chlororibose, 2′-deoxy-3′-aminoribose, 2′-deoxy-3′-(C1-C6)alkylribose, 2′-deoxy-3′-(C1-C6)alkoxyribose and 2′-deoxy-3′-(C5-C14)aryloxyribose, ribose, 2′-deoxyribose, 2′,3′-dideoxyribose, 2′-haloribose, 2′-fluororibose, 2′-chlororibose, and 2′-alkylribose, e.g., 2′-O-methyl, 4′-α-anomeric nucleotides, 1′-α-anomeric nucleotides, 2′-4′- and 3′-4′-linked and other “locked” or “LNA”, bicyclic sugar modifications (see, e.g., PCT Published Application Nos. WO 98/22489, WO 98/39352, and WO 99/14226). Exemplary LNA sugar analogs within a polynucleotide include, but are not limited to, the structures: 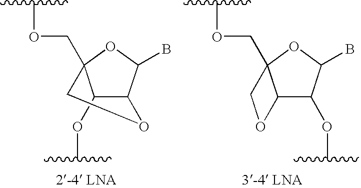
where B is any nucleotide base.

Modifications at the 2′- or 3′-position of ribose include, but are not limited to, hydrogen, hydroxy, methoxy, ethoxy, allyloxy, isopropoxy, butoxy, isobutoxy, methoxyethyl, alkoxy, phenoxy, azido, amino, alkylamino, fluoro, chloro and bromo. Nucleotides include, but are not limited to, the natural D optical isomer, as well as the L optical isomer forms (see, e.g., Garbesi et al., (1993) Nucl. Acids Res. 21:4159-65; Fujimori et al., (1990) J. Amer. Chem. Soc. 112:7436-38; Urata et al., (1993) Nucleic Acids Symposium Ser. No. 29:69-70). When the nucleotide base is a purine, e.g. A or G, the ribose sugar is attached to the N⁹-position of the nucleotide base. When the nucleotide base is a pyrimidine, e.g. C, T or U, the pentose sugar is attached to the N′-position of the nucleotide base, except for pseudouridines, in which the pentose sugar is attached to the C5 position of the uracil nucleotide base (see, e.g., Kornberg and Baker, (1992) DNA Replication, 2 Ed., Freeman, San Francisco, Calif.).

One or more of the pentose carbons of a nucleotide may be substituted with a phosphate ester having the formula: 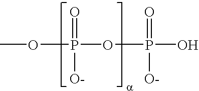
where α is an integer from 0 to 4. In certain embodiments, α is 2 and the phosphate ester is attached to the 3′- or 5′-carbon of the pentose. In certain embodiments, the nucleotides are those in which the nucleotide base is a purine, a 7-deazapurine, a pyrimidine, or an analog thereof. “Nucleotide 5′-triphosphate” refers to a nucleotide with a triphosphate ester group at the 5′ position, and are sometimes denoted as “NTP”, or “dNTP” and “ddNTP” to particularly point out the structural features of the ribose sugar. The triphosphate ester group may include sulfur substitutions for the various oxygens, e.g. α-thio-nucleotide 5′-triphosphates. For a review of nucleotide chemistry, see, e.g., Shabarova, Z. and Bogdanov, A. Advanced Organic Chemistry of Nucleic Acids, VCH, New York, 1994.

The term “nucleotide analog” refers to embodiments in which the pentose sugar and/or the nucleotide base and/or one or more of the phosphate esters of a nucleotide may be replaced with its respective analog. In certain embodiments, exemplary pentose sugar analogs are those described above. In certain embodiments, the nucleotide analogs have a nucleotide base analog as described above. In certain embodiments, exemplary phosphate ester analogs include, but are not limited to, alkylphosphonates, methylphosphonates, phosphoramidates, phosphotriesters, phosphorothioates, phosphorodithioates, phosphoroselenoates, phosphorodiselenoates, phosphoroanilothioates, phosphoroanilidates, phosphoroamidates, boronophosphates, etc., and may include associated counterions;

Also included within the definition of “nucleotide analog” are nucleotide analog monomers which can be polymerized into polynucleotide analogs in which the DNA/RNA phosphate ester and/or the sugar phosphate ester backbone is replaced with a different type of internucleotide linkage. Exemplary polynucleotide analogs include, but are not limited to, peptide nucleic acids, in which the sugar phosphate backbone of the polynucleotide is replaced by a peptide backbone.

An “extendable nucleotide” is a nucleotide which is: (i) capable of being enzymatically or synthetically incorporated onto the terminus of a polynucleotide chain, and

(ii) capable of supporting further enzymatic or synthetic extension. Extendable nucleotides include nucleotides that have already been enzymatically or synthetically incorporated into a polynucleotide chain, and have either supported further enzymatic or synthetic extension, or are capable of supporting further enzymatic or synthetic extension. Extendable nucleotides include, but are not limited to, nucleotide 5′-triphosphates, e.g., dNTP and NTP, phosphoramidites suitable for chemical synthesis of polynucleotides, and nucleotide units in a polynucleotide chain that have already been incorporated enzymatically or chemically, but do not include nucleotide terminators.

The term “nucleotide terminator” or “terminator” refers to an enzymatically-incorporable nucleotide, which does not support incorporation of subsequent nucleotides in a primer extension reaction. A terminator is therefore not an extendable nucleotide. In certain embodiments, terminators are those in which the nucleotide is a purine, a 7-deaza-purine, a pyrimidine, or a nucleotide analog, and the sugar moiety is a pentose which includes a 3′-substituent that blocks further synthesis, such as a dideoxynucleotide triphosphate (ddNTP). In certain embodiments, substituents that block further synthesis include, but are not limited to, amino, deoxy, halogen, alkoxy and aryloxy groups. Exemplary terminators include, but are not limited to, those in which the sugar-phosphate ester moiety is 3′-(C1-C6)alkylribose-5′-triphosphate, 2′-deoxy-3′-(C1-C6)alkylribose-5′-triphosphate, 2′-deoxy-3′-(C1-C6)alkoxyribose-5-triphosphate, 2′-deoxy-3′-(C5-C14)aryloxyribose-5′-triphosphate, 2′-deoxy-3′-haloribose-5′-triphosphate, 2′-deoxy-3′-aminoribose-5′-triphosphate, 2′,3′-dideoxyribose-5′-triphosphate or 2′,3′-didehydroribose-5′-triphosphate. Terminators include, but are not limited to, T terminators, including ddTTP and ddUTP, which incorporate opposite an adenine, or adenine analog, in a template; A terminators, including ddATP, which incorporate opposite a thymine, uracil, or an analog of thymine or uracil, in the template; C terminators, including ddCTP, which incorporate opposite a guanine, or guanine analog, in the template; and G terminators, including ddGTP and ddITP, which incorporate opposite a cytosine, or cytosine analog, in the template.

As used herein, the terms “polynucleotide”, “oligonucleotide”, and “nucleic acid” are used interchangeably and refer to single-stranded and double-stranded polymers of nucleotide monomers, including 2′-deoxyribonucleotides (DNA) and ribonucleotides (RNA) linked by internucleotide phosphodiester bond linkages, or internucleotide analogs, and associated counter ions, e.g., H⁺, NH₄⁺, trialkylammonium, Mg²⁺, Na⁺, and the like. A polynucleotide may be composed entirely of deoxyribonucleotides, entirely of ribonucleotides, or chimeric mixtures thereof. The nucleotide monomer units may comprise any of the nucleotides described herein, including, but not limited to, nucleotides and nucleotide analogs. Polynucleotides typically range in size from a few monomeric units, e.g. 5-40 when they are sometimes referred to in the art as oligonucleotides, to several thousands of monomeric nucleotide units. Unless denoted otherwise, whenever a polynucleotide sequence is represented, it will be understood that the nucleotides are in 5′ to 3′ order from left to right and that “A” denotes deoxyadenosine or an analog thereof, “C” denotes deoxycytidine or an analog thereof, “G” denotes deoxyguanosine or an analog thereof, and “T” denotes thymine or an analog thereof, unless otherwise noted.

Polynucleotides may be composed of a single type of sugar moiety, e.g., as in the case of RNA and DNA, or mixtures of different sugar moieties, e.g., as in the case of RNA/DNA chimeras. In certain embodiments, nucleic acids are ribopolynucleotides and 2′-deoxyribopolynucleotides according to the structural formulae below: 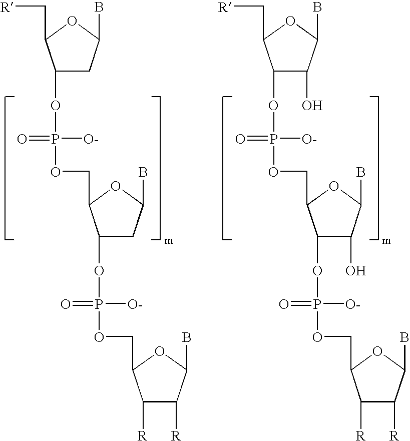
wherein each B is independently the base moiety of a nucleotide, e.g., a purine, a 7-deazapurine, a pyrimidine, or an analog thereof; each m defines the length of the respective nucleic acid and can range from zero to thousands, tens of thousands, or even more; each R is independently selected from the group comprising hydrogen, hydroxyl, halogen, —R″, —OR″, and —NR″R″, where each R″ is independently (C₁-C₆) alkyl or (C₅-C1₄) aryl, or two adjacent Rs may be taken together to form a bond such that the ribose sugar is 2′,3′-didehydroribose, and each R′ may be independently hydroxyl or 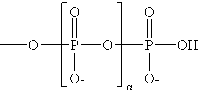
where α is zero, one or two.

In certain embodiments of the ribopolynucleotides and 2′-deoxyribopolynucleotides illustrated above, the nucleotide bases B are covalently attached to the C1′ carbon of the sugar moiety as previously described.

The terms “nucleic acid”, “polynucleotide”, and “oligonucleotide” may also include nucleic acid analogs, polynucleotide analogs, and oligonucleotide analogs. The terms “nucleic acid analog”, “polynucleotide analog” and “oligonucleotide analog” are used interchangeably, and refer to a polynucleotide that contains at least one nucleotide analog and/or at least one phosphate ester analog and/or at least one pentose sugar analog. A polynucleotide analog may comprise one or more lesions. Also included within the definition of polynucleotide analogs are polynucleotides in which the phosphate ester and/or sugar phosphate ester linkages are replaced with other types of linkages, such as N-(2-aminoethyl)-glycine amides and other amides (see, e.g., Nielsen et al., 1991, Science 254: 1497-1500; WO 92/20702; U.S. Pat. No. 5,719,262; U.S. Pat. No. 5,698,685;); morpholinos (see, e.g., U.S. Pat. No. 5,698,685; U.S. Pat. No. 5,378,841; U.S. Pat. No. 5,185,144); carbamates (see, e.g., Stirchak & Summerton, 1987, J. Org. Chem. 52: 4202); methylene(methylimino) (see, e.g., Vasseur et al., 1992, J. Am. Chem. Soc. 114: 4006); 3′-thioformacetals (see, e.g., Jones et al., 1993, J. Org. Chem. 58: 2983); sulfamates (see, e.g., U.S. Pat. No. 5,470,967); 2-aminoethylglycine, commonly referred to as PNA (see, e.g., Buchardt, WO 92/20702; Nielsen (1991) Science 254:1497-1500); and others (see, e.g., U.S. Pat. No. 5,817,781; Frier & Altman, 1997, Nucl. Acids Res. 25:4429 and the references cited therein). Phosphate ester analogs include, but are not limited to, (i) C₁-C₄ alkylphosphonate, e.g. methylphosphonate; (ii) phosphoramidate; (iii) C₁-C₆ alkyl-phosphotriester; (iv) phosphorothioate; and (v) phosphorodithioate.

An “enzymatically active mutant or variant thereof,” when used in reference to an enzyme such as a polymerase or a ligase, means a protein with appropriate enzymatic activity. Thus, for example, but without limitation, an enzymatically active mutant or variant of a DNA polymerase is a protein that is able to catalyze the stepwise addition of appropriate deoxynucleoside triphosphates into a nascent DNA strand in a template-dependent manner. An enzymatically active mutant or variant differs from the “generally-accepted” or consensus sequence for that enzyme by at least one amino acid, including, but not limited to, substitutions of one or more amino acids, addition of one or more amino acids, deletion of one or more amino acids, and alterations to the amino acids themselves. With the change, however, at least some catalytic activity is retained. In certain embodiments, the changes involve conservative amino acid substitutions. Conservative amino acid substitution may involve replacing one amino acid with another that has, e.g., similar hydrophobicity, hydrophilicity, charge, or aromaticity. In certain embodiments, conservative amino acid substitutions may be made on the basis of similar hydropathic indices. A hydropathic index takes into account the hydrophobicity and charge characteristics of an amino acid, and in certain embodiments, may be used as a guide for selecting conservative amino acid substitutions. The hydropathic index is discussed, e.g., in Kyte et al., J. Mol. Biol., 157:105-131 (1982). It is understood in the art that conservative amino acid substitutions may be made on the basis of any of the aforementioned characteristics.

Alterations to the amino acids may include, but are not limited to, glycosylation, methylation, phosphorylation, biotinylation, and any covalent and noncovalent additions to a protein that do not result in a change in amino acid sequence. “Amino acid” as used herein refers to any amino acid, natural or non-natural, that may be incorporated, either enzymatically or synthetically, into a polypeptide or protein.

Fragments, for example, but without limitation, proteolytic cleavage products, are also encompassed by this term, provided that at least some enzyme catalytic activity is retained.

The skilled artisan will readily be able to measure catalytic activity using an appropriate well-known assay. Thus, an appropriate assay for polymerase catalytic activity might include, for example, measuring the ability of a variant to incorporate, under appropriate conditions, rNTPs or dNTPs into a nascent polynucleotide strand in a template-dependent manner. Likewise, an appropriate assay for ligase catalytic activity might include, for example, the ability to ligate adjacently hybridized oligonucleotides comprising appropriate reactive groups. Protocols for such assays may be found, among other places, in Sambrook et al., Molecular Cloning, A Laboratory Manual, Cold Spring Harbor Press (1989) (hereinafter “Sambrook et al.”), Sambrook and Russell, Molecular Cloning, Third Edition, Cold Spring Harbor Press (2000) (hereinafter “Sambrook and Russell”), Ausubel et al., Current Protocols in Molecular Biology (1993) including supplements through May 2005, John Wiley & Sons (hereinafter “Ausubel et al.”).

The term “methylation state” refers to the presence or absence of a methyl group on a particular nucleotide.

A “target” or “target nucleic acid sequence” is a nucleic acid sequence in a sample. In certain embodiments, a target nucleic acid sequence serves as a template for amplification in a PCR reaction. In certain embodiments, a target nucleic acid sequence serves as a ligation template. Target nucleic acid sequences may include both naturally occurring and synthetic molecules.

Different target nucleic acid sequences may be different portions of a single contiguous nucleic acid or may be on different nucleic acids. Different portions of a single contiguous nucleic acid may overlap.

The term “target nucleotide” means a nucleotide that can be distinguished by a probe and/or primer. In certain embodiments, the type of nucleotide is distinguished. In certain embodiments, the methylation state of the nucleotide is distinguished. In certain embodiments, both the type of nucleotide and the methylation state are distinguished.

The term “target cytosine” means a target nucleotide of a target nucleic acid sequence that is a cytosine. In certain embodiments, the methylation state of a target cytosine is sought to be determined.

A target nucleic acid sequence may comprise one or more lesions. In certain embodiments, a target nucleic acid sequence comprising one or more lesions is called a “lesion-containing target nucleic acid sequence.” Lesions include, but are not limited to, one or more nucleotides with at least one abnormal alteration in its chemical properties, e.g., a base alteration, a base deletion, a sugar alteration, or an alteration which causes a strand break. Specifically, lesions include, but are not limited to, abasic sites; AAF adducts, including, but not limited to, N-(deoxyguanosine-8-yl)-2-acetylaminofluorene and N-(deoxyguanosine-8-yl)-2-aminofluorene; cis-cyn pyrimidine dimers (also referred to as cyclobutane pyrimidine dimers), including, but not limited to, cis-syn thymine-thymine dimers; 6-4 pyrimidine-pyrimidone dimers; benzo[a]pyrene diol epoxide adducts, including, but not limited to, benzo[a]pyrene diol epoxide deoxyadenosine adducts and benzo[a]pyrene diol epoxide deoxyguanosine adducts; oxidized guanine, including, but not limited to, 7,8-dihydro-8-oxoguanine, and 8-oxoguanine, (8-hydroxyguanine); oxidized adenine, including, but not limited to, 7,8-dihydro-8-oxoadenine, and 8-oxoadenine, (8-hydroxyadenine); 5-hydroxycytosine; 5-hydroxyuracil; 5,6-dihydouracil; cisplatin adducts, including but not limited to, 1,2-cisplatinated guanine; 5,6-dihydro-5,6-dihyroxythymine (thymine glycol); 1,N⁶-ethenodeoxyadenosine; O⁶-methylguanine; cyclodeoxyadenosine; 2,6-diamino-4-hydroxyformamidopyrimidine; 8-nitroguanine; N²-guanine monoadducts of 1,3-butadiene metabolites; and oxidized cytosine.

Lesions also include, but are not limited to, any alteration in a polynucleotide resulting from radiation, oxidative damage, and chemical mutagens. Sources of radiation include, but are not limited to, nonionizing radiation (e.g., UV radiation), or ionizing radiation (e.g., X-rays, gamma radiation, and corpuscular radiation (e.g., α-particle and β-particle radiation)). Sources of oxidative damage include, but are not limited to, oxidative damage mediated by one or more transition metals (e.g., the combination of H₂O₂ and CuCl₂)), and chemical mutagens. Chemical mutagens include, but are not limited to, base analogs (e.g., bromouracil or aminopurine), chemicals which alter the structure and pairing properties of bases (e.g., nitrous acid, nitrosoguanidine, methyl methanesulfonate (MMS), and ethyl methanesulfonate (EMS)), intercalating agents (e.g., ethidium bromide, acridine orange, and proflavin), agents altering DNA structure (e.g., large molecules that bind to bases in DNA and cause them to be noncoding (e.g., acetyl aminofluorene (AAF), N-acetoxy-2-aminofluorene (NAAAF), or cisplatin), agents causing inter- and intrastrand crosslinks (e.g., psoralens), methylated and acetylated bases, and chemicals causing DNA strand breaks (e.g., peroxides)).

The term “microsatellite” refers to a repetitive stretch of a short sequence of DNA. In certain embodiments, the short sequence of DNA is two bases in length. In certain embodiments, the short sequence of DNA is three bases in length. In certain embodiments, the short sequence of DNA is four bases in length. In certain embodiments, the short sequence of DNA is more than four bases in length. In certain embodiments, microsatellites include short tandem repeats (STRs). In certain embodiments, microsatellites can be used as genetic markers.

The term “genotype” refers to the specific allelic composition of one or more genes of an organism. The term “genotyping” refers to testing that reveals certain specific alleles carried by an individual.

The term “sample” refers to any substance comprising nucleic acid material.

In certain embodiments, a sample may contain a mixture of target nucleic acid sequences, some of which are methylated at a particular target nucleotide and some of which are not methylated at that target nucleotide. As used herein, the term “degree of methylation” refers to the relative number of target nucleic acid sequences within a sample that are methylated at a target nucleotide, compared to those that are not methylated at that target nucleotide.

The term “combined methylation” refers to the degree of methylation of two or more different target nucleotides in a sample. In certain embodiments, the term “overall degree of methylation” includes the degree of methylation of all of the nucleotides in a sample.

The term “addressable portion” refers to a nucleic acid sequence designed to hybridize to the complement of the addressable portion. For a pair of addressable portions that are complementary to one another, one member will be called an addressable portion and the other will be called a complementary addressable portion.

The term “probe” comprises a polynucleotide that comprises a specific portion designed to hybridize in a sequence-specific manner with a complementary region of a specific nucleic acid sequence, e.g., a target polynucleotide. In certain embodiments, the specific portion of the probe may be specific for a particular sequence, or alternatively, may be degenerate, e.g., specific for a set of sequences.

The term “ligation probe set” refers to a group of two or more probes designed to detect at least one target. As a non-limiting example, a ligation probe set may comprise two nucleic acid probes designed to hybridize to a target such that, when the two probes are hybridized to the target adjacent to one another, they are suitable for ligation together.

When used in the context of the present teachings, “suitable for ligation” refers to at least one first ligation probe and at least one second ligation probe, each comprising an appropriately reactive group. Exemplary reactive groups include, but are not limited to, a free hydroxyl group on the 3′ end of the first probe and a free phosphate group on the 5′ end of the second probe. Exemplary pairs of reactive groups include, but are not limited to: phosphorothioate and tosylate or iodide; esters and hydrazide; RC(O)S⁻, haloalkyl, or RCH₂S and α-haloacyl; thiophosphoryl and bromoacetoamido groups. Exemplary reactive groups include, but are not limited to, S-pivaloyloxymethyl-4-thiothymidine. Additionally, in certain embodiments, first and second ligation probes are hybridized to the target sequence such that the 3′ end of the first ligation probe and the 5′ end of the second ligation probe are immediately adjacent to allow ligation.

The “proximal end” of a probe of a ligation probe set refers to the end of a nucleic acid probe that is designed to hybridize adjacent to another nucleic acid probe of the ligation probe set. In certain embodiments, the proximal ends of two nucleic acid probes are suitable for ligation together when hybridized to a target nucleic acid sequence.

The term “interaction probe” refers to a probe that comprises at least two moieties that can interact with one another to provide a detectably different signal value depending upon whether a given nucleic acid sequence is present or absent. The signal value that is detected from the interaction probe is different depending on whether the two moieties are sufficiently close to one another or are spaced apart from one another. In certain embodiments employing interaction probes, the proximity of the two moieties to one another is different depending upon whether the given nucleic acid sequence is present or absent.

The term “5′-nuclease probe,” refers to a probe which comprises a signal moiety linked to a quencher moiety or a donor moiety through a short oligonucleotide link element. When the 5′-nuclease probe is intact, the quencher moiety or the donor moiety influences the detectable signal from the signal moiety. According to certain embodiments, the 5′-nuclease probe binds to a specific nucleic acid sequence, and is cleaved by the 5′ nuclease activity of at least one of a polymerase and another enzymatic construct when the probe is replaced by a newly polymerized strand during an amplification reaction such as PCR or some other strand displacement protocol.

When the oligonucleotide link element of the 5′-nuclease probe is cleaved, the detectable signal from the signal moiety changes when the signal moiety becomes further separated from the quencher moiety or the donor moiety. In certain such embodiments that employ a quencher moiety, the signal value increases when the signal moiety becomes further separated from the quencher moiety. In certain such embodiments that employ a donor moiety, the signal value decreases when the signal moiety becomes further separated from the donor moiety.

The term “detectable signal value” refers to a value of the signal that is detected from a label. In certain embodiments, the detectable signal value is the amount or intensity of signal that is detected from a label. Thus, if there is no detectable signal value from a label, its detectable signal value is zero (0). In certain embodiments, the detectable signal value is a characteristic of the signal other than the amount or intensity of the signal, such as the spectra, wavelength, color, or lifetime of the signal.

“Detectably different signal” means that detectable signals from different labels are distinguishable from one another by at least one detection method.

“Detectably different signal value” means that one or more detectable signal values are distinguishable from one another by at least one detection method.

The term “threshold difference between detectable signal values” refers to a set difference between a first detectable signal value and a second detectable signal value that results when the target nucleic acid sequence that is being sought is present in a sample, but that does not result when the target nucleic acid sequence is absent. The first detectable signal value of a labeled probe is the detectable signal value from the probe when it is not exposed to a given nucleic acid sequence. The second detectable signal value is detected during and/or after an amplification reaction using a composition that comprises the labeled probe.

The term “quencher moiety” refers to a moiety that causes the signal value of a signal moiety to differ depending on whether the quencher moiety is sufficiently close to the signal moiety or is spaced apart from the signal moiety. In certain embodiments, the quencher moiety decreases the detectable signal value from the signal moiety when the quencher moiety is sufficiently close to the signal moiety. In certain embodiments, the quencher moiety decreases the detectable signal value to zero or close to zero when the quencher moiety is sufficiently close to the signal moiety.

The term “labeled probe” refers to a probe that comprises a label.

The term “variable signal value probe” refers to a probe that provides a detectably different signal value depending upon whether a given nucleic acid sequence is present or absent. In certain embodiments, a variable signal value probe provides a detectably different signal value when the intact variable signal value probe is hybridized to a given nucleic acid sequence than when the intact variable signal value probe is not hybridized to a given nucleic acid sequence. Thus, if a given nucleic acid sequence is present, the variable signal value probe provides a detectably different signal value than when the given nucleic acid sequence is absent. In certain embodiments, a variable signal value probe provides a detectably different signal value when the probe is intact than when the probe is not intact. In certain such embodiments, a variable signal value probe remains intact unless a given nucleic acid sequence is present. In certain such embodiments, if a given nucleic acid sequence is present, the variable signal value probe is cleaved, which results in a detectably different signal value than when the probe is intact.

The term “double-stranded-dependent label” refers to a label that provides a detectably different signal value when it is exposed to double-stranded nucleic acid than when it is not exposed to double-stranded nucleic acid.

The term “quantitating,” when used in reference to an amplification product, refers to determining the quantity, amount, or relative quantity of a particular sequence that is representative of a target nucleic acid sequence in the sample. For example, but without limitation, one may measure the intensity of the signal from a label. The intensity or quantity of the signal is typically related to the amount of amplification product. The amount of amplification product generated correlates with the amount of target nucleic acid sequence present prior to ligation and amplification, and thus, in certain embodiments, may indicate the level of expression for a particular gene.

The terms “annealing” and “hybridization” are used interchangeably and mean the base-pairing interaction of one nucleic acid with another nucleic acid that results in formation of a duplex, triplex, or other higher-ordered structure. In certain embodiments, the primary interaction is base specific, e.g., A/T and G/C, by Watson/Crick and Hoogsteen-type hydrogen bonding. In certain embodiments, base-stacking and hydrophobic interactions may also contribute to duplex stability.

The term “amplification product” as used herein refers to the product of an amplification reaction including, but not limited to, primer extension, the polymerase chain reaction, RNA transcription, and the like. Thus, exemplary amplification products may comprise at least one of primer extension products, PCR amplicons, RNA transcription products, and the like.

The term “primer” refers to a polynucleotide that anneals to a target polynucleotide and allows the synthesis from its 3′ end of a sequence complementary to the target polynucleotide.

A “universal primer” is capable of hybridizing to the primer-specific portion (or its complement) of more than one species of probe, ligation product, or amplification product, as appropriate. A “universal primer set” comprises a first primer and a second primer that hybridize with a plurality of species of probes, ligation products, or amplification products, as appropriate.

A “ligation agent” according to the present teachings may comprise any number of enzymatic or chemical (i.e., non-enzymatic) agents that can effect ligation of nucleic acids to one another.

In this application, a statement that one sequence is the same as or is complementary to another sequence encompasses situations where both of the sequences are completely the same or complementary to one another, and situations where only a portion of one of the sequences is the same as, or is complementary to, a portion or the entire other sequence. Here, the term “sequence” encompasses, but is not limited to, nucleic acid sequences, polynucleotides, oligonucleotides, probes, primers, primer-specific portions, addressable portions, and target-specific portions.

In this application, a statement that one sequence is complementary to another sequence encompasses situations in which the two sequences have mismatches. Here, the term “sequence” encompasses, but is not limited to, nucleic acid sequences, polynucleotides, oligonucleotides, probes, primers, primer-specific portions, addressable portions, and target-specific portions. Despite the mismatches, the two sequences should selectively hybridize to one another under appropriate conditions.

The term “selectively hybridize” means that, for particular identical sequences, a substantial portion of the particular identical sequences hybridize to a given desired sequence or sequences, and a substantial portion of the particular identical sequences do not hybridize to other undesired sequences. A “substantial portion of the particular identical sequences” in each instance refers to a portion of the total number of the particular identical sequences, and it does not refer to a portion of an individual particular identical sequence. In certain embodiments, “a substantial portion of the particular identical sequences” means at least 70% of the particular identical sequences. In certain embodiments, “a substantial portion of the particular identical sequences” means at least 80% of the particular identical sequences. In certain embodiments, “a substantial portion of the particular identical sequences” means at least 90% of the particular identical sequences. In certain embodiments, “a substantial portion of the particular identical sequences” means at least 95% of the particular identical sequences.

In certain embodiments, the number of mismatches that may be present may vary in view of the complexity of the composition. Thus, in certain embodiments, the more complex the composition, the more likely undesired sequences will hybridize. For example, in certain embodiments, with a given number of mismatches, a probe may more likely hybridize to undesired sequences in a composition with the entire genomic DNA than in a composition with fewer DNA sequences, when the same hybridization and wash conditions are employed for both compositions. Thus, that given number of mismatches may be appropriate for the composition with fewer DNA sequences, but fewer mismatches may be more optimal for the composition with the entire genomic DNA.

In certain embodiments, sequences are complementary if they have no more than 20% mismatched nucleotides. In certain embodiments, sequences are complementary if they have no more than 15% mismatched nucleotides. In certain embodiments, sequences are complementary if they have no more than 10% mismatched nucleotides. In certain embodiments, sequences are complementary if they have no more than 5% mismatched nucleotides.

In this application, a statement that one sequence hybridizes or binds to another sequence encompasses situations where the entirety of both of the sequences hybridize or bind to one another, and situations where only a portion of one or both of the sequences hybridizes or binds to the entire other sequence or to a portion of the other sequence. Here, the term “sequence” encompasses, but is not limited to, nucleic acid sequences, polynucleotides, oligonucleotides, probes, primers, primer-specific portions, addressable portions, and target-specific portions.

In certain embodiments, the term “to a measurably lesser extent” encompasses situations in which the event in question is reduced at least 10 fold. In certain embodiments, the term “to a measurably lesser extent” encompasses situations in which the event in question is reduced at least 100 fold.

In certain embodiments, a statement that a component may be, is, or has been “substantially removed” means that at least 90% of the component may be, is, or has been removed. In certain embodiments, a statement that a component may be, is, or has been “substantially removed” means that at least 95% of the component may be, is, or has been removed.

The term “mismatch hybridization” refers to hybridization between two nucleic acids where at least one mismatch is present in the hybridized nucleic acids. The term “mismatch” refers to naturally-occurring bases that align, but that are not an A-T, A-U, or G-C base pair.

The term “mismatch discrimination” refers to the ability of a probe to hybridize with greater affinity to a first nucleic acid sequence than to a second nucleic acid sequence, where the second nucleic acid sequence/probe duplex comprises more mismatches than the first nucleic acid sequence/probe duplex.

The term “mismatch ligation” refers to a ligation reaction where a ligation product is formed even though at least one mismatch exists between at least one of the probes of the ligation probe set and the target nucleic acid sequence.

A “blocking probe” is a probe that hybridizes to a portion of a nucleic acid sequence and blocks hybridization of at least one probe of a ligation probe set to the portion of the nucleic acid sequence. In certain embodiments, a blocking probe competes with at least one of the ligation probes of the ligation probe set for hybridization to nucleic acid sequences. In certain such embodiments, the blocking probe differs from the competing ligation probes in the ligation probe set by at least one nucleotide. In certain such embodiments, the at least one nucleotide difference results in the blocking probe binding to a nucleic acid sequence to which binding of the competing ligation probe is not desired. In certain such embodiments, the blocking probe is designed to block mismatch ligation by specifically binding to the nucleic acid sequence to which binding of the competing ligation probe is not desired and blocking mismatch hybridization of the competing ligation probe to that nucleic acid sequence. In certain embodiments, a blocking probe comprises sufficient sequence specificity to hybridize to a modified nucleic acid sequence, but does not hybridize to the unmodified version of that nucleic acid sequence.

In certain embodiments, target nucleic acid sequences are treated with a modifying agent. The term “modifying agent” refers to any agent that can modify a nucleic acid.

In certain embodiments, a modifying agent converts a target nucleotide into a different nucleotide. The different nucleotide that results from this conversion is called a “converted nucleotide”. For example, and not limitation, in certain embodiments, a modifying agent converts cytosine to uracil. In those embodiments, uracil is a converted nucleotide.

“Mobility modifiers” mean any moieties that effect a particular mobility of a polynucleotide in a mobility-dependent analysis technique, such as electrophoresis.

“Mobility-dependent analysis technique” refers to any analysis based on different rates of migration between different analytes. Exemplary mobility-dependent analyses include, but are not limited to, electrophoresis, mass spectroscopy, chromatography, sedimentation, gradient centrifugation, field-flow fractionation, and multi-stage extraction techniques.

The term “capture moiety” means any molecule that can be used to at least partially isolate a nucleic acid. In certain embodiments, the term “capture moiety” includes affinity sets.

As used herein, an “affinity set” is a set of molecules that specifically bind to one another. Affinity sets include, but are not limited to, biotin and avidin, biotin and streptavidin, receptor and ligand, antibody and ligand, antibody and antigen, and a polynucleotide sequence and its complement. One or more members of an affinity set may be coupled to a solid support. Exemplary solid supports include, but are not limited to, beads, agarose, sepharose, magnetic beads, polystyrene, polyacrylamide, glass, membranes, silica, semiconductor materials, silicon, and organic polymers.

Certain Exemplary Components

Exemplary target nucleic acid sequences include, but are not limited to, RNA and DNA. Exemplary RNA target sequences include, but are not limited to, mRNA, rRNA, tRNA, snRNA, viral RNA, and variants of RNA, such as splicing variants. Exemplary DNA target sequences include, but are not limited to, genomic DNA, plasmid DNA, phage DNA, nucleolar DNA, mitochondrial DNA, chloroplast DNA, cDNA, synthetic DNA, yeast artificial chromosomal DNA (“YAC”), bacterial artificial chromosome DNA (“BAC”), other extrachromosomal DNA, and primer extension products. Target nucleic acid sequences also include, but are not limited to, analogs of both RNA and DNA. Exemplary nucleic acid analogs include, but are not limited to, locked nucleic acids (“LNAs”), peptide nucleic acids (“PNAs”), 8-aza-7-deazaguanine (“PPG's”), and other nucleic acid analogs. In certain embodiments, target nucleic acid sequences include chimeras of RNA and DNA.

A variety of methods are available for obtaining certain target nucleic acid sequences for use with the compositions and methods of certain embodiments. When the nucleic acid target is obtained through isolation from a biological matrix, certain isolation techniques include, but are not limited to, (1) organic extraction followed by ethanol precipitation, e.g., using a phenol/chloroform organic reagent (e.g., Ausubel et al., eds., Current Protocols in Molecular Biology Volume 1, Chapter 2, Section I, John Wiley & Sons, New York (1993)), in certain embodiments, using an automated DNA extractor, e.g., the Model 341 DNA Extractor available from Applied Biosystems (Foster City, Calif.); (2) stationary phase adsorption methods (e.g., Boom et al., U.S. Pat. No. 5,234,809; Walsh et al., Biotechniques 10(4): 506-513 (1991)); and (3) salt-induced DNA precipitation methods (e.g., Miller et al., Nucleic Acids Research, 16(3): 9-10 (1988)), such precipitation methods being typically referred to as “salting-out” methods. In certain embodiments, the above isolation methods may be preceded by an enzyme digestion step to help eliminate unwanted protein from the sample, e.g., digestion with proteinase K, or other like proteases. See, e.g., U.S. patent application Ser. No. 09/724,613.

In certain embodiments, a target nucleic acid sequence may be derived from any living, or once living, organism, including but not limited to, prokaryotes, eukaryotes, including plants and animals, and viruses. Animals from which a target nucleic acid sequence may be derived include worms and flies. In certain embodiments, the target nucleic acid sequence may originate from a nucleus of a cell, e.g., genomic DNA, or may be extranuclear nucleic acid, e.g., plasmid, mitrochondrial nucleic acid, various RNAs, and the like. In certain embodiments, if the sequence from the organism is RNA, it may be reverse-transcribed into a cDNA target nucleic acid sequence. Furthermore, in certain embodiments, the target nucleic acid sequence may be present in a double stranded or single stranded form.

Exemplary target nucleic acid sequences include, but are not limited to, amplification products, ligation products, transcription products, reverse transcription products, primer extension products, methylated DNA, and cleavage products. Exemplary amplification products include, but are not limited to, PCR products and isothermal amplification products.

In certain embodiments, nucleic acids in a sample may be subjected to a cleavage procedure. In certain embodiments, such cleavage products may be targets.

In certain embodiments, a target nucleic acid sequence may be derived from a crude cell lysate, a clinical sample, or a forensic sample. Examples of target nucleic acid sequences include, but are not limited to, nucleic acids from buccal swabs, crude bacterial lysates, blood, skin, semen, hair, and bone.

In certain embodiments, a target nucleic acid sequence may comprise one or more forensic markers. The term “forensic marker” refers to one or more characteristics which can be used to distinguish a first nucleic acid from a second nucleic acid. In certain embodiments, one or more forensic markers can be used to distinguish the source of a first nucleic acid from the source of a second nucleic acid. In certain embodiments, a single forensic marker can be used to distinguish the source of a first nucleic acid from the source of a second nucleic acid. In certain embodiments, two or more forensic markers can be used to distinguish the source of a first nucleic acid from the source of a second nucleic acid.

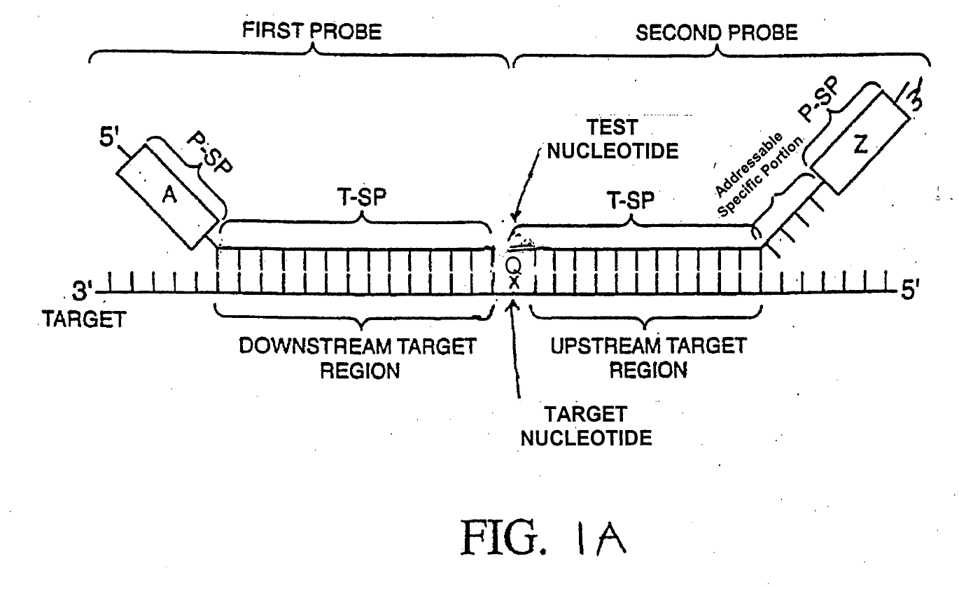

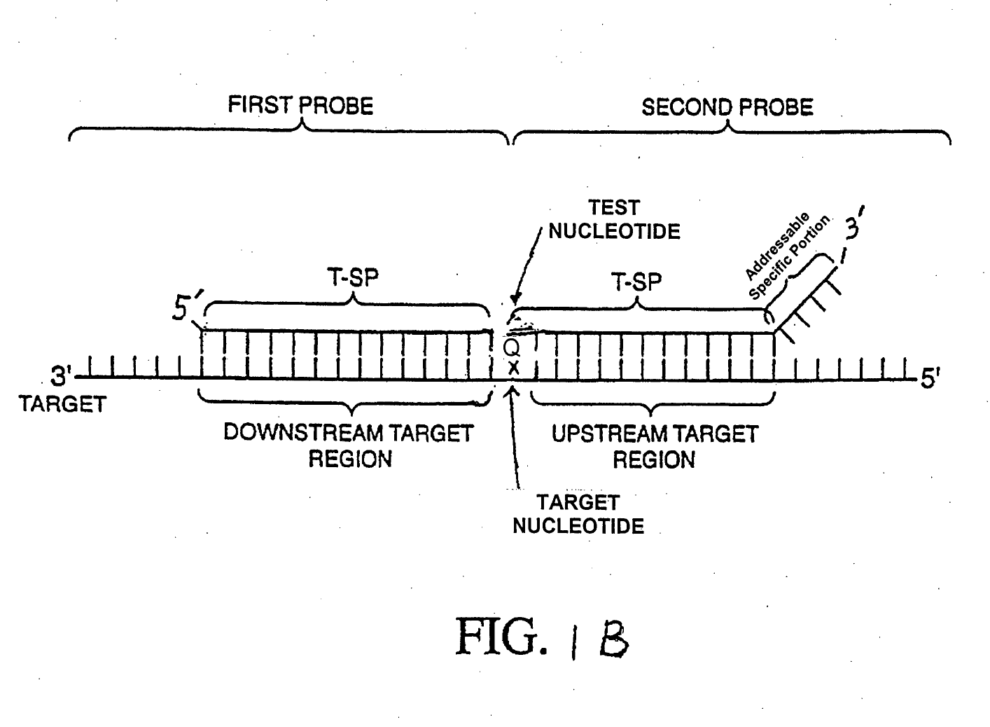

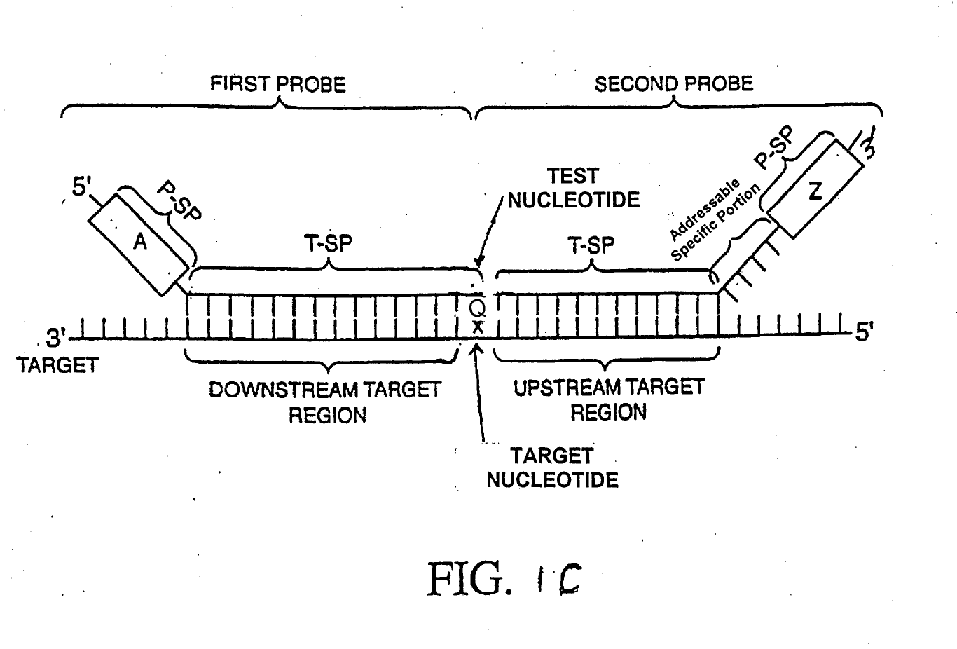

In certain embodiments, a target nucleic acid sequence comprises an upstream or 5′ region, a downstream or 3′ region, and a target nucleotide located in the upstream region or the downstream region (see, e.g., FIGS. 1(A)-(D). In certain embodiments, the target nucleotide may be the nucleotide being detected by the ligation probe set to determine the methylation state of a target cytosine. In certain embodiments, more than one target nucleotide is present. In certain embodiments, one or more target nucleotides are located in the upstream region, and one or more target nucleotides are located in the downstream region. In certain embodiments, more than one target nucleotide is located in the upstream region or the downstream region.

In certain embodiments, a target nucleic acid sequence is treated with one or more modifying agents. Non-limiting examples of compounds that may serve as suitable modifying agents include bisulfite compounds, for example but not limited to, sodium bisulfite, magnesium bisulfite, manganese bisulfite, potassium bisulfite, ammonium bisulfite; 5-bromouracil; and certain sulfhydryl compounds, for example but not limited to, mercaptoethanol, cysteine methyl ester, glutathione, and cysteamine. Descriptions of exemplary modifying agents can be found in, among other places, Hayatsu, Prog. Nuc. Acid Res. Mol. Biol. 16:75-124, 1975; Hayatsu, Proc. Japanese Acad. Ser. B, 80:189-94, 2004; Boyd and Zon, Anal. Biochem. 326:278-80, 2004; U.S. patent application Ser. No. 10/926,530; and U.S. Published Patent Application No. US 2005-008989A1).

In certain embodiments, one may subject an initial sample comprising a target nucleic acid sequence to an amplification reaction to increase the amount of target nucleic acid sequence to which probes in a ligation probe set will hybridize. In certain embodiments, amplification is carried out as described in U.S. Provisional Patent Application No. 60/654,192.

The person of ordinary skill will appreciate that while a target nucleic acid sequence is typically described as a single-stranded molecule, the opposing strand of a double-stranded molecule comprises a complementary sequence that may also be used as a target sequence.

A ligation probe set, according to certain embodiments, comprises two or more probes that each comprise a target-specific portion that is designed to hybridize in a sequence-specific manner with a complementary region on a specific target nucleic acid sequence (see, e.g., first and second probes in FIGS. 1(A)-(D)). In certain embodiments, a probe of a ligation probe set may further comprise a primer-specific portion, an addressable portion, all or part of a promoter or its complement, or a combination of these additional components. In certain embodiments, any of the probe's components may overlap any other probe component(s). For example, but without limitation, the target-specific portion may overlap the primer-specific portion, the promoter or its complement, or both. Also, without limitation, the addressable portion may overlap with the target-specific portion or the primer specific-portion, or both.

In certain embodiments, at least one probe of a ligation probe set comprises an addressable portion located between a target-specific portion and a primer-specific portion (see, e.g., second probe in FIGS. 1(A) and 1(C)). In certain embodiments, a probe's addressable portion may comprise a sequence that is the same as, or complementary to, a portion of a capture oligonucleotide sequence located on an addressable support or a bridging oligonucleotide. In certain embodiments, the probe's addressable portion may comprise a mobility modifier that allows detection of the ligation or amplification products based on their location at a particular mobility address due to a mobility detection process, such as, but without limitation, electrophoresis. In certain embodiments, one employs a mobility-modifier comprising (1) a complementary addressable portion or addressable portion for selectively binding to the addressable portion or complementary addressable portion of a ligation product and/or an amplification product, and (2) a tail for effecting a particular mobility in a mobility-dependent analysis technique, e.g., electrophoresis, see, e.g., U.S. Pat. No. 6,395,486. In certain embodiments, a probe's addressable portion is not complementary with target nucleic acid sequences, primer sequences, or probe sequences.

The sequence-specific portions of probes are of sufficient length to permit specific annealing to complementary sequences in primers, mobility modifier cassettes, and targets as appropriate. In certain embodiments, the length of the addressable portions are 6 to 35 nucleotides. In certain embodiments, the length of the addressable portions are greater than 35 nucleotides. In certain embodiments, the length of the addressable portions are less than 6 nucleotides. In certain embodiments, the length of the target-specific portions are 6 to 35 nucleotides. In certain embodiments, the length of the target-specific portions are greater than 35 nucleotides. In certain embodiments, the length of the target-specific portions are less than 6 nucleotides. In certain embodiments, the length of the primer-specific portions are 6 to 35 nucleotides. In certain embodiments, the length of the primer-specific portions are greater than 35 nucleotides. In certain embodiments, the length of the primer-specific portions are less than 6 nucleotides.

A ligation probe set according to certain embodiments comprises at least one first probe and at least one second probe that are designed to adjacently hybridize to the same target nucleic acid sequence. According to certain embodiments, a ligation probe set is designed so that the target-specific portion of the first probe will hybridize with the downstream target region (see, e.g., FIGS. 1(A)-(D)) and the target-specific portion of the second probe will hybridize with the upstream target region (see, e.g., FIGS. 1(A)-(D)). The sequence-specific portions of the probes are of sufficient length to permit specific annealing with complementary sequences in targets and primers, as appropriate. In certain embodiments, one of the at least one first probe and the at least one second probe in a ligation probe set further comprises an addressable portion.

Under appropriate conditions, adjacently hybridized probes may be ligated together to form a ligation product, provided that they comprise appropriate reactive groups, for example, without limitation, a free 3′-hydroxyl or 5′-phosphate group.

According to certain embodiments, some ligation probe sets may comprise more than one first probe or more than one second probe to allow sequence discrimination between target sequences that differ by one or more nucleotides.

In certain embodiments, a nucleotide base complementary to the target nucleotide (X), the “test nucleotide” is present on the proximal end of the second probe of the ligation probe set (see, e.g., 5′ end (Q) of the second probe in FIGS. 1(A) and (B)). In certain embodiments, the second probe further comprises an addressable portion (see, e.g., FIGS. 1(A) and (B)). In certain embodiments, the first probe may comprise a test nucleotide and the second probe may comprise an addressable portion (see, e.g., FIG. 1(C)). In certain embodiments, the first probe may comprise a test nucleotide and an addressable portion. In certain embodiments, the second probe may comprise a test nucleotide and the first probe may comprise an addressable portion.

The skilled artisan will appreciate that the target nucleotide(s) may be located anywhere in the target sequence and that likewise, the test nucleotide may be located anywhere within the target-specific portion of the probe(s). For example, according to various embodiments, the test nucleotide may be located at the 3′ end of a probe, at the 5′ end of a probe, or anywhere between the 3′ end and the 5′ end of a probe. Furthermore, in certain embodiments, two or more test nucleotides may be located on a probe.

In certain embodiments, when the first and second probes of the ligation probe set are hybridized to the appropriate upstream and downstream target regions, and when the test nucleotide is at the 5′ end of one probe or the 3′ end of the other probe, and the test nucleotide is base-paired with the target nucleotide on the target sequence, the hybridized first and second probes may be ligated together to form a ligation product. In certain embodiments, a mismatched base at the test nucleotide, however, interferes with ligation, even if both probes are otherwise fully hybridized to their respective target regions.

In certain embodiments, other mechanisms may be employed to avoid ligation of probes that do not include the correct complementary nucleotide at the test nucleotide. For example, in certain embodiments, conditions may be employed such that a probe of a ligation probe set will hybridize to the target sequence to a measurably lesser extent if there is a mismatch at the target nucleotide. Thus, in such embodiments, such non-hybridized probes will not be ligated to the other probe in the ligation probe set.

In certain embodiments, the first probes and second probes in a ligation probe set are designed with similar melting temperatures (Tₘ). Where a probe includes a test nucleotide, in certain embodiments, the Tₘ for the probe(s) comprising the test nucleotide(s) will be approximately 4-15° C. lower than the other probe(s) that do not contain the test nucleotide in the ligation probe set. In certain such embodiments, the probe comprising the test nucleotide(s) will also be designed with a Tₘ near the ligation temperature. Thus, a probe with a mismatched nucleotide will more readily dissociate from the target at the ligation temperature. The ligation temperature, therefore, in certain embodiments provides another way to discriminate between, for example, target cytosine methylation states in the target.

In certain embodiments, the Tₘ of the blocking probe is about the same as the Tₘ of the probes in a ligation probe set. In certain embodiments, the Tₘ of the blocking probe is higher than the Tₘ of the probes in a ligation probe set. In certain embodiments, the Tₘ of the blocking probe is lower than the Tₘ of the probes in a ligation probe set. In certain embodiments, by varying the Tₘ of the blocking probe relative to the ligation probe sets, one can control the effectiveness with which the blocking probe blocks at least one of the probes of the ligation probe set from binding to a particular target nucleic acid sequence.

Further, in certain embodiments, ligation probe sets do not comprise a test nucleotide at the terminus of the first or the second probe (e.g., at the 3′ end or the 5′ end of the first or second probe). Rather, the test nucleotide is located somewhere between the 5′ end and the 3′ end of the first or second probe. In certain such embodiments, probes with target-specific portions that are fully complementary with their respective target regions will hybridize under high stringency conditions. Probes with one or more mismatched bases in the target-specific portion, by contrast, will hybridize to their respective target region to a measurably lesser extent. Both the first probe and the second probe must be hybridized to the target for a ligation product to be generated.

In certain embodiments, one of the first probe or the second probe may contain a test nucleotide and the other of the first probe or the second probe may contain an addressable portion.

In certain embodiments, one of the first or second probes of a ligation probe set may include an addressable portion and the first and second probes may not include primer-specific portions. In certain such embodiments, at least one first probe of a ligation probe set comprises a test nucleotide and an addressable portion and at least one second probe of a ligation probe set comprises a label. In certain embodiments, at least one first probe of a ligation probe set comprises a test nucleotide and a label and at least one second probe of a ligation probe set comprises an addressable portion.

In certain embodiments, a blocking probe comprises at least one modification that enhances mismatch discrimination. Exemplary modifications that enhance mismatch discrimination include, but are not limited to, minor groove binder groups. Examples of minor groove binder groups that can be used to enhance mismatch discrimination are found, e.g., in Kutyavin et al., Nucleic Acids Research, 28(2): 655-661 (2000). In certain embodiments, a modification that enhances mismatch discrimination will either (a) increase the affinity of the blocking probe for a nucleic acid sequence that is complementary to the blocking probe sequence without any mismatches, (b) decrease the affinity of the blocking probe for a nucleic acid sequence that differs by at least one nucleotide from a sequence that is complementary to the blocking probe without any mismatches, or (c) both increase the affinity of the blocking probe for a nucleic acid sequence that is complementary to the blocking probe sequence without any mismatches and decrease the affinity of the blocking probe for a nucleic acid sequence that differs by at least one nucleotide from a sequence that is complementary to the blocking probe without any mismatches.

In certain embodiments, a blocking probe comprises LNA, PNA, or other polynucleotide modifications to enhance mismatch discrimination.

In certain embodiments, a blocking probe comprises a modified guanine. In certain embodiments, modified guanines will not base pair with methylated cytosine, but will base pair with unmethylated cytosine.

In certain embodiments, conditions are employed such that at least one probe will hybridize to the target sequence to a measurably lesser extent if the target nucleotide is not in the appropriate methylation state. In certain embodiments, conditions are employed such that at least one probe will not hybridize to the target sequence if the target nucleotide is not in the appropriate methylation state.

In certain embodiments, conditions are employed such that probes of a ligation probe set will ligate together to a measurably lesser extent if the target nucleotide is not in the appropriate methylation state, even if the probes of the ligation probe set are hybridized to the target sequence. In certain embodiments, conditions are employed such that probes of a ligation probe set will not ligate together if the target nucleotide is not in the appropriate methylation state, even if the probes of the ligation probe set are hybridized to the target sequence.

In certain embodiments, conditions are employed such that at least one probe will hybridize to the target sequence to a measurably lesser extent if the target nucleotide is methylated. In certain embodiments, conditions are employed such that at least one probe will not hybridize to the target sequence if the target nucleotide is methylated.

In certain embodiments, conditions are employed such that probes of a ligation probe set will ligate together to a measurably lesser extent if the target nucleotide is methylated, even if the probes of the ligation probe set are hybridized to the target sequence. In certain embodiments, conditions are employed such that probes of a ligation probe set will not ligate together if the target nucleotide is methylated, even if the probes of the ligation probe set are hybridized to the target sequence.

In certain embodiments, conditions are employed such that at least one probe will hybridize to the target sequence to a measurably lesser extent if the target nucleotide is unmethylated. In certain embodiments, conditions are employed such that at least one probe will not hybridize to the target sequence if the target nucleotide is unmethylated.

In certain embodiments, conditions are employed such that probes of a ligation probe set will ligate together to a measurably lesser extent if the target nucleotide is unmethylated, even if the probes of the ligation probe set are hybridized to the target sequence. In certain embodiments, conditions are employed such that probes of a ligation probe set will not ligate together if the target nucleotide is unmethylated, even if the probes of the ligation probe set are hybridized to the target sequence.

In certain embodiments, conditions are employed such that at least one probe will hybridize to the target sequence to a measurably lesser extent if the target nucleotide is modified. In certain embodiments, conditions are employed such that at least one probe will not hybridize to the target sequence if the target nucleotide is modified.

In certain embodiments, conditions are employed such that probes of a ligation probe set will ligate together to a measurably lesser extent if the target nucleotide is modified, even if the probes of the ligation probe set are hybridized to the target sequence. In certain embodiments, conditions are employed such that probes of a ligation probe set will not ligate together if the target nucleotide is modified, even if the probes of the ligation probe set are hybridized to the target sequence.

In certain embodiments, conditions are employed such that at least one probe will hybridize to the target sequence to a measurably lesser extent if the target nucleotide is unmodified. In certain embodiments, conditions are employed such that at least one probe will not hybridize to the target sequence unless the target nucleotide is modified.

In certain embodiments, conditions are employed such that probes of a ligation probe set will ligate together to a measurably lesser extent if the target nucleotide is unmodified, even if the probes of the ligation probe set are hybridized to the target sequence. In certain embodiments, conditions are employed such that probes of a ligation probe set will not ligate together unless the target nucleotide is modified, even if the probes of the ligation probe set are hybridized to the target sequence.

In certain embodiments, conditions are employed such that at least one probe will hybridize to the target sequence to a measurably lesser extent if the target nucleotide is converted to a converted nucleotide. In certain embodiments, conditions are employed such that at least one probe will not hybridize to the target sequence if the target nucleotide is converted to a converted nucleotide.

In certain embodiments, conditions are employed such that probes of a ligation probe set will ligate together to a measurably lesser extent if the target nucleotide is converted to a converted nucleotide, even if the probes of the ligation probe set are hybridized to the target sequence. In certain embodiments, conditions are employed such that probes of a ligation probe set will not ligate together if the target nucleotide is converted to a converted nucleotide, even if the probes of the ligation probe set are hybridized to the target sequence.

In certain embodiments, conditions are employed such that at least one probe will hybridize to the target sequence to a measurably lesser extent if the target nucleotide has not been converted to a converted nucleotide. In certain embodiments, conditions are employed such that at least one probe will not hybridize to the target sequence unless the target nucleotide is converted to a converted nucleotide.

In certain embodiments, conditions are employed such that probes of a ligation probe set will ligate together to a measurably lesser extent if the target nucleotide has not been converted to a converted nucleotide, even if the probes of the ligation probe set are hybridized to the target sequence. In certain embodiments, conditions are employed such that probes of a ligation probe set will not ligate together unless the target nucleotide is converted to a converted nucleotide, even if the probes of the ligation probe set are hybridized to the target sequence.

A primer set according to certain embodiments comprises at least one primer capable of hybridizing with the primer-specific portion of at least one probe of a ligation probe set. In certain embodiments, a primer set comprises at least one first primer and at least one second primer, wherein the at least one first primer specifically hybridizes with one probe of a ligation probe set (or a complement of such a probe) and the at least one second primer of the primer set specifically hybridizes with a second probe of the same ligation probe set (or a complement of such a probe). In certain embodiments, at least one primer of a primer set further comprises all or part of a promoter sequence or its complement. In certain embodiments, the first and second primers of a primer set have different hybridization temperatures, to permit temperature-based asymmetric PCR reactions.

The skilled artisan will appreciate that while the probes and primers of the present teachings may be described in the singular form, a plurality of probes or primers can be encompassed by the singular term, as will be apparent from the context. Thus, for example, in certain embodiments, a ligation probe set typically comprises a plurality of first probes and a plurality of second probes.

The criteria for designing sequence-specific primers and probes are well known to persons of ordinary skill in the art. Detailed descriptions of primer design that provide for sequence-specific annealing can be found, among other places, in Diffenbach and Dveksler, PCR Primer, A Laboratory Manual, Cold Spring Harbor Press, 1995, and Kwok et al. (Nucl. Acid Res. 18:999-1005, 1990). The sequence-specific portions of the primers are of sufficient length to permit specific annealing to complementary sequences in ligation products and amplification products, as appropriate.

According to certain embodiments, a primer set comprises at least one second primer. The second primer in that primer set is designed to hybridize with a 3′ primer-specific portion of a ligation or amplification product in a sequence-specific manner. In certain embodiments, the primer set further comprises at least one first primer. The first primer of a primer set is designed to hybridize with the complement of the 5′ primer-specific portion of that same ligation or amplification product in a sequence-specific manner. In certain embodiments, at least one primer of the primer set comprises a promoter sequence or its complement or a portion of a promoter sequence or its complement. For a discussion of primers comprising promoter sequences, see, e.g., Sambrook and Russell. In certain embodiments, at least one primer of the primer set further comprises a label. In certain embodiments, labels are fluorescent dyes attached to a nucleotide(s) in the primer (see, e.g., L. Kricka, Nonisotopic DNA Probe Techniques, Academic Press, San Diego, Calif. (1992)). In certain embodiments, a label is attached to the primer in such a way as not to interfere with sequence-specific hybridization or amplification.

A universal primer or primer set may be employed according to certain embodiments. In certain embodiments, a universal primer or a universal primer set hybridizes with all or most of the probes, ligation products, or amplification products in a reaction, as appropriate. When universal primer sets are used in certain amplification reactions, such as, but not limited to, PCR, qualitative or quantitative results may be obtained for a broad range of template concentrations.

The term “label” refers to any molecule that can be detected. In certain embodiments, a label can be a moiety that produces a signal or that interacts with another moiety to produce a signal. In certain embodiments, a label can interact with another moiety to modify a signal of the other moiety. In certain embodiments, a label can bind to another moiety or complex that produces a signal or that interacts with another moiety to produce a signal. In certain embodiments, the label emits a detectable signal only when the probe is bound to a complementary target nucleic acid sequence. In certain embodiments, the label emits a detectable signal only when the label is cleaved from the polynucleotide probe. In certain embodiments, the label emits a detectable signal only when the label is cleaved from the polynucleotide probe by a 5′ exonuclease reaction.

Exemplary, labels include, but are not limited to, light-emitting or light-absorbing compounds which generate or quench a detectable fluorescent, chemiluminescent, or bioluminescent signal (see, e.g., Kricka, L. in Nonisotopic DNA Probe Techniques (1992), Academic Press, San Diego, pp. 3-28). Fluorescent reporter dyes useful as labels include, but are not limited to, fluoresceins (see, e.g., U.S. Pat. Nos. 5,188,934; 6,008,379; and 6,020,481), rhodamines (see, e.g., U.S. Pat. Nos. 5,366,860; 5,847,162; 5,936,087; 6,051,719; and 6,191,278), benzophenoxazines (see, e.g., U.S. Pat. No. 6,140,500), energy-transfer fluorescent dyes, comprising pairs of donors and acceptors (see, e.g., U.S. Pat. Nos. 5,863,727; 5,800,996; and 5,945,526), and cyanines (see, e.g., Kubista, WO 97/45539), as well as any other fluorescent moiety capable of generating a detectable signal. Examples of fluorescein dyes include, but are not limited to, 6-carboxyfluorescein; 2′,4′,1,4,-tetrachlorofluorescein; and 2′,4′,5′,7′,1,4-hexachlorofluorescein.

Exemplary labels include, but are not limited to, quantum dots. “Quantum dots” refer to semiconductor nanocrystalline compounds capable of emitting a second energy in response to exposure to a first energy. Typically, the energy emitted by a single quantum dot has the same predictable wavelength. Exemplary semiconductor nanocrystalline compounds include, but are not limited to, crystals of CdSe, CdS, and ZnS. Suitable quantum dots according to certain embodiments are described, e.g., in U.S. Pat. Nos. 5,990,479 and 6,207,392 B1, and in “Quantum-dot-tagged microbeads for multiplexed optical coding of biomolecules,” Han et al., Nature Biotechnology, 19:631-635 (2001).

Exemplary labels include, but are not limited to, phosphors and luminescent molecules, fluorophores, radioisotopes, chromogens, enzymes, antigens, heavy metals, dyes, magnetic probes, phosphorescence groups, chemiluminescent groups, and electrochemical detection moieties. Exemplary fluorophores include, but are not limited to, rhodamine, cyanine 3 (Cy 3), cyanine 5 (Cy 5), fluorescein, Vic™, LiZ™, Tamra™, 5-Fam™, 6-Fam™, and Texas Red (Molecular Probes). (Vic™, LiZ™, Tamra™, 5-Fam™, and 6-Fam™ are all available from Applied Biosystems, Foster City, Calif.) Exemplary radioisotopes include, but are not limited to, ³²P, ³³P, and ³⁵S. Exemplary labels also include elements of multi-element indirect reporter systems, e.g., biotin/avidin, antibody/antigen, ligand/receptor, enzyme/substrate, and the like, in which the element interacts with other elements of the system in order to effect a detectable signal. One exemplary multi-element reporter system includes a biotin reporter group attached to a primer and an avidin conjugated with a fluorescent label.

The skilled artisan will appreciate that, in certain embodiments, one or more of the primers, probes, deoxyribonucleotide triphosphates, ribonucleotide triphosphates disclosed herein may further comprise one or more labels. Exemplary detailed protocols for methods of attaching labels to oligonucleotides and polynucleotides can be found in, among other places, G. T. Hermanson, Bioconjugate Techniques, Academic Press, San Diego, Calif. (1996) and S. L. Beaucage et al., Current Protocols in Nucleic Acid Chemistry, John Wiley & Sons, New York, N.Y. (2000).

Certain exemplary non-radioactive labeling methods, techniques, and reagents are reviewed in: Non-Radioactive Labelling, A Practical Introduction, Garman, A. J. (1997) Academic Press, San Diego.

In certain embodiments, a mobility modifier may be employed. In certain embodiments, mobility modifiers may be nucleotides of different lengths effecting different mobilities. In certain embodiments, mobility modifiers may be non-nucleotide polymers, such as a polyethylene oxide (PEO), polyglycolic acid, polyurethane polymers, polypeptides, or oligosaccharides, as non-limiting examples. In certain embodiments, mobility modifiers may work by adding size to a polynucleotide, or by increasing the “drag” of the molecule during migration through a medium without substantially adding to the size. Certain mobility modifiers such as PEO's have been described, e.g., in U.S. Pat. Nos. 5,470,705; 5,580,732; 5,624,800; and 5,989,871.

Certain linkage of polymers such as PEO's to polynucleotides is well known in the art. Standard DNA chemistry linkages are described, e.g., in Grossman et al., Nucleic Acids Research, 22(21):4527-34 (1994).

Certain embodiments include a ligation agent. For example, ligase is an enzymatic ligation agent that, under appropriate conditions, forms phosphodiester bonds between the 3′-OH and the 5′-phosphate of adjacent nucleotides in DNA or RNA molecules, or hybrids. Exemplary ligases include, but are not limited to, Tth K294R ligase and Tsp AK16D ligase. See, e.g., Luo et al., Nucleic Acids Res., 24(14):3071-3078 (1996); Tong et al., Nucleic Acids Res., 27(3):788-794 (1999); and Published PCT Application No. WO 00/26381. Exemplary temperature sensitive ligases, include, but are not limited to, T4 DNA ligase, T7 DNA ligase, and E. coli ligase. Exemplary thermostable ligases include, but are not limited to, Taq ligase, Tth ligase, Tsc ligase, and Pfu ligase. Certain thermostable ligases may be obtained from thermophilic or hyperthermophilic organisms, including but not limited to, prokaryotic, eucaryotic, or archael organisms. Certain RNA ligases may be employed in certain embodiments. In certain embodiments, the ligase is a RNA dependent DNA ligase, which may be employed with RNA template and DNA ligation probes. An exemplary, but nonlimiting example, of a ligase with such RNA dependent DNA ligase activity is T4 DNA ligase. In certain embodiments, the ligation agent is an “activating” or reducing agent.

Exemplary chemical ligation agents include, without limitation, activating, condensing, and reducing agents, such as carbodiimide, cyanogen bromide (BrCN), N-cyanoimidazole, imidazole, 1-methylimidazole/carbodiimide/cystamine, dithiothreitol (DTT) and ultraviolet light. Autoligation, i.e., spontaneous ligation in the absence of a ligating agent, is also within the scope of certain embodiments of the present teachings. Detailed protocols for certain chemical ligation methods and descriptions of appropriate reactive groups can be found, among other places, in Xu et al., Nucleic Acid Res., 27:875-81 (1999); Gryaznov and Letsinger, Nucleic Acid Res. 21:1403-08 (1993); Gryaznov et al., Nucleic Acid Res. 22:2366-69 (1994); Kanaya and Yanagawa, Biochemistry 25:7423-30 (1986); Luebke and Dervan, Nucleic Acids Res. 20:3005-09 (1992); Sievers and von Kiedrowski, Nature 369:221-24 (1994); Liu and Taylor, Nucleic Acids Res. 26:3300-04 (1999); Wang and Kool, Nucleic Acids Res. 22:2326-33 (1994); Purmal et al., Nucleic Acids Res. 20:3713-19 (1992); Ashley and Kushlan, Biochemistry 30:2927-33 (1991); Chu and Orgel, Nucleic Acids Res. 16:3671-91 (1988); Sokolova et al., FEBS Letters 232:153-55 (1988); Naylor and Gilham, Biochemistry 5:2722-28 (1966); and U.S. Pat. No. 5,476,930.

In certain embodiments, at least one polymerase is included. In certain embodiments, at least one thermostable polymerase is included. Exemplary thermostable polymerases, include, but are not limited to, Taq polymerase, Pfx polymerase, Pfu polymerase, Vent® polymerase, Deep Vent™ polymerase, Pwo polymerase, Tth polymerase, UITma polymerase and enzymatically active mutants and variants thereof. Descriptions of these polymerases may be found, among other places, at the world wide web URL: the-scientist.com/yr1998/jan/profile1_{—}980105.html; at the world wide web URL: the-scientist.com/yr2001/jan/profile_{—}010903.html; at the world wide web URL: the-scientist.com/yr2001/sep/profile2_{—}010903.html; at the article The Scientist 12(1):17 (Jan. 5, 1998); and at the article The Scientist 15(17):1 (Sep. 3, 2001).

The skilled artisan will appreciate that the complement of the disclosed probe, target, and primer sequences, or combinations thereof, may be employed in certain embodiments of the present teachings. For example, without limitation, a genomic DNA sample may comprise both the target sequence and its complement. Thus, in certain embodiments, when a genomic sample is denatured, both the target sequence and its complement are present in the sample as single-stranded sequences. In certain embodiments, ligation probes may be designed to specifically hybridize to an appropriate sequence, either the target sequence or its complement.

Certain Exemplary Component Methods

Ligation according to the present teachings comprises any enzymatic or chemical process wherein an internucleotide linkage is formed between the opposing ends of nucleic acid sequences. In certain embodiments, the nucleic acid sequences are adjacently hybridized to a template such that their opposing ends are proximal. Additionally, the opposing ends of the annealed nucleic acid sequences should be suitable for ligation (suitability for ligation is a function of the ligation method employed). The internucleotide linkage may include, but is not limited to, phosphodiester bond formation. Such bond formation may include, without limitation, those created enzymatically by a DNA or RNA ligase, such as bacteriophage T4 DNA ligase, T4 RNA ligase, T7 DNA ligase, Thermus thermophilus (Tth) ligase, Thermus aquaticus (Taq) ligase, TspAK16D ligase, or Pyrococcus furiosus (Pfu) ligase. Other internucleotide linkages include, without limitation, covalent bond formation between appropriate reactive groups such as between an (α-haloacyl group and a phosphothioate group to form a thiophosphorylacetylamino group; and between a phosphorothioate and a tosylate or iodide group to form a 5′-phosphorothioester or pyrophosphate linkages.

In certain embodiments, chemical ligation may, under appropriate conditions, occur spontaneously such as by autoligation. Alternatively, in certain embodiments, “activating,” condensing, or reducing agents may be used. Examples of activating agents, condensing agents, and reducing agents include, without limitation, carbodiimide, cyanogen bromide (BrCN), imidazole, 1-methylimidazole/carbodiimide/cystamine, N-cyanoimidazole, and dithiothreitol (DTT) (see, e.g., Xu et al., Nucl. Acids Res. 27:875-81, 1999; Gryaznov and Letsinger, Nucl. Acids Res. 21: 1403-08, 1993; Gryaznov et al., Nucleic Acid Res. 22:2366-69, 1994; Kanaya and Yanagawa, Biochemistry 25:7423-30, 1986; Luebke and Dervan, Nucl. Acids Res. 20:3005-09, 1992; Sievers and von Kiedrowski, Nature 369:221-24, 1994; Liu and Taylor, Nucl. Acids res. 26:3300-04, 1999; Wang and Kool, Nucl. Acids Res. 22:2326-33, 1994; Purmal et al., Nucl. Acids Res. 20:3713-19, 1992; Ashley and Kushlan, Biochemistry 30:2927-33, 1991; Chu and Orgel, Nucl. Acids Res. 16:3671-91, 1988; Sokolova et al., FEBS Letters 232:153-55, 1988; Naylor and Gilham, Biochemistry 5:2722-28, 1966; Hames and Ellington, Chem. & Biol. 4:595-605, 1997; and U.S. Pat. No. 5,476,930). Non-enzymatic ligation according to certain embodiments may utilize specific reactive groups on the respective 3′ and 5′ ends of the aligned probes. In certain embodiments, chemical ligation may occur by photoligation. Photoligation includes, but is not limited to: probes comprising nucleotide analogs, including but not limited to, 4-thiothymidine (s4T), 5-vinyluracil and its derivatives, or combination thereof; light in the UV-A range (about 320 nm to about 400 nm); light in the UV-B range (about 290 nm to about 320 nm); combinations of light in the UV-A and UV-B range; light with a wavelength between about 300 nm and about 375 nm; light with a wavelength of about 360 nm to about 370 nm; light with a wavelength of about 364 nm to about 368 nm; and/or light with a wavelength of about 366 nm. In certain embodiments, photoligation is reversible. Descriptions of photoligation can be found in, for example, Fujimoto et al., Nucl. Acid Symp. Ser. 42:39-40, 1999; Fujimoto et al., Nucl. Acid Res. Suppl. 1: 185-86, 2001; Fujimoto et al., Nucl. Acid. Suppl. 2: 155-56, 2002; and Liu and Taylor, Nucl. Acid Res. 26: 3300-04, 1998.

In certain embodiments, ligation generally comprises at least one cycle of ligation, for example, the sequential procedures of: hybridizing the target-specific portions of a first probe and a second probe, that are suitable for ligation, to their respective complementary regions on a target nucleic acid sequence; ligating the 3′ end of the first probe with the 5′ end of the second probe to form a ligation product; and denaturing the nucleic acid duplex to separate the ligation product from the target nucleic acid sequence; The cycle may or may not be repeated. For example, without limitation in certain embodiments, thermocycling the ligation reaction may be employed to linearly increase the amount of ligation product.

According to certain embodiments, one may use ligation techniques such as gap-filling ligation, including, without limitation, gap-filling OLA and LCR, bridging oligonucleotide ligation, FEN-LCR, and correction ligation. Descriptions of these techniques can be found, among other places, in U.S. Pat. No. 5,185,243, published European Patent Applications EP 320308 and EP 439182, published PCT Patent Application WO 90/01069, published PCT Patent Application WO 02/02823, and U.S. Pat. No. 6,511,810.

In certain embodiments, ligation also comprises at least one gap-filling procedure, wherein the ends of the two ligation probes are not adjacently hybridized initially but the 3′-end of the first ligation probe is extended by one or more nucleotides until it is adjacent to the 5′-end of the second ligation probe by a DNA polymerase. Thus, the ligation probes become hybridized adjacent to one another on the target nucleic acid sequence. In certain other embodiments, there is a gap between the 3′-end of the first probe and the 5′-end of the second probe such that a ‘gap oligonucleotide’ can hybridize in the gap between the ends of the two probes, for example, to increase specificity. In some embodiments, the 3′-end of the first probe can be ligated to the 5′-end of the gap oligonucleotide and the 3′-end of the gap oligonucleotide can be ligated to the 5′-end of the second probe. Thus, the ligation probes become hybridized adjacent to one another on the target nucleic acid sequence through the gap oligonucleotide.

In certain embodiments, one may employ poly-deoxy-inosinic-deoxy-cytidylic acid (Poly [d(I-C)]) (Available in Roche Applied Science catalog, 2002) in a ligation reaction. In certain embodiments, one uses any number between 15 to 80 ng/μL of Poly [d(I-C)] in a ligation reaction. In certain embodiments, one uses 30 ng/μL of Poly [d(I-C)] in a ligation reaction.

One may use Poly [d(I-C)] in a ligation reaction with various methods employing ligation probes discussed herein. In certain embodiments, one may use Poly [d(1-C)] with different types of ligation methods. For example, one may use Poly [d(I-C)] in any of a variety of methods employing ligation reactions. Exemplary methods include, but are not limited to, those discussed in U.S. Pat. No. 6,027,889, PCT Published Patent Application No. WO 01/92579, and U.S. Patent Application Publication 2004-0121371.

Exemplary, but nonlimiting ligation reaction conditions may be as follows. In certain embodiments, the ligation reaction temperature may range anywhere from about 45° C. to 55° C. for anywhere from two to 10 minutes. In certain embodiments, any number from 2 to 100 cycles of ligation are performed. In certain embodiments, 60 cycles of ligation are performed. In certain embodiments, allele specific ligation probes (a probe of a ligation probe set that is specific to a particular allele at a given locus) are in a concentration anywhere from 2 to 100 nM. In certain embodiments, allele specific ligation probes are in a concentration of 50 nM. In certain embodiments, allele specific ligation probes are in a concentration anywhere from 1 to 7 nM. In certain embodiments, the locus specific ligation probes (a probe of a ligation probe set that is not specific to a particular allele, but is specific for a given locus) are in a concentration anywhere from 2 to 200 nM. In certain embodiments, locus specific ligation probes are in a concentration of 100 nM. In certain embodiments, fragmented genomic DNA is in a concentration anywhere from 5 ng/:l to 200 ng/:l in the ligation reaction. In certain embodiments, fragmented genomic DNA is in a concentration of 130 ng/:l in the ligation reaction. In certain embodiments, the pH for the ligation reaction is anywhere from 7 to 8. In certain embodiments, the Mg++concentration is anywhere from 2 to 22 nM. In certain embodiments, the ligase concentration is anywhere from 0.04 to 0.16 u/:l. In certain embodiments, the ligase concentration is anywhere from 0.02 to 0.12 u/:l. In certain embodiments, the K+ concentration is anywhere from 0 to 70 mM. In certain embodiments, the K+ concentration is anywhere from 0 to 20 mM. In certain embodiments, the Poly [d(I-C)] concentration is anywhere from 0 to 30 ng/:l. In certain embodiments, the Poly [d(I-C)] concentration is anywhere from 0 to 20 ng/:l. In certain embodiments, the NAD+concentration is anywhere from 0.25 to 2.25 mM.

In certain embodiments, one forms a test composition for a subsequent amplification reaction by subjecting a ligation reaction composition to at least one cycle of ligation. In certain embodiments, after ligation, the test composition may be used directly in the subsequent amplification reaction. In certain embodiments, prior to the amplification reaction, the test composition may be subjected to a purification technique that results in a “purified” test composition that includes less than all of the components that may have been present after the at least one cycle of ligation.

Purifying the ligation product according to certain embodiments comprises any process that removes at least some unligated probes, target nucleic acid sequences, enzymes, and/or accessory agents from the ligation reaction composition following at least one cycle of ligation. Such processes include, but are not limited to, molecular weight/size exclusion processes, e.g., gel filtration chromatography or dialysis, sequence-specific hybridization-based pullout methods, affinity capture techniques, precipitation, adsorption, or other nucleic acid purification techniques. The skilled artisan will appreciate that purifying the ligation product prior to amplification in certain embodiments reduces the quantity of primers needed to amplify the ligation product, thus reducing the cost of detecting a target nucleic acid sequence. In certain embodiments, purifying the ligation product prior to amplification may decrease possible side reactions during amplification and may reduce competition from unligated probes during hybridization.

Hybridization-based pullout (HBP) according to certain embodiments comprises a process wherein a nucleotide sequence complementary to at least a portion of one probe (or its complement), for example, the primer-specific portion, is bound or immobilized to a solid or particulate pullout support (see, e.g., U.S. Pat. No. 6,124,092). In certain embodiments, a composition comprising ligation product, target sequences, and unligated probes is exposed to the pullout support. The ligation product, under appropriate conditions, hybridizes with the support-bound sequences. In certain embodiments, the unbound components of the composition are removed, purifying the ligation products from those ligation reaction composition components that do not contain sequences complementary to the sequence on the pullout support. One subsequently removes the purified ligation products from the support and combines them with at least one primer set to form a first amplification reaction composition. The skilled artisan will appreciate that, in certain embodiments, additional cycles of HBP using different complementary sequences on the pullout support may remove all or substantially all of the unligated probes, further purifying the ligation product.

In certain embodiments, one may substantially remove certain unligated probes employing a ligation probe set that includes a binding moiety on either the 5′ end of the first probe or the 3′ end of the second probe. In certain such embodiments, after a ligation reaction, one exposes the composition to a support that binds to the binding moiety. In certain embodiments, the unbound components of the composition are removed, substantially purifying the ligation products from those ligation reaction composition components that do not include the binding moiety, including the unligated probes without a binding moiety. In certain such embodiments, one may then remove the bound components from the support, and then expose them to a support with a bound sequence that is complementary to a portion of the ligation probe without the binding moiety, and that is not complementary to a portion of the ligation probe with the binding moiety. Thus, in certain such embodiments, the unligated first and second probes will be substantially removed from the ligation product. In certain embodiments, one may reverse the process by exposing the composition first to the support with the complementary sequence and second to the support that binds to the binding moiety. In certain embodiments, the binding moiety is biotin, which binds to streptavidin on the support.

In certain embodiments, one may employ different binding moieties (e.g., a first binding moiety and a second binding moiety) on the first probe and second probe of a ligation probe set. In certain such embodiments, after a ligation reaction, one may then expose the composition to a first support that binds one of the binding moieties to capture ligation product and unligated probe with the first binding moiety. In certain embodiments, after removing unbound components, one may then remove the bound components and expose them to a second support that binds the second binding moiety to capture ligation product.

In certain embodiments, one may substantially remove unligated ligation probes using certain exonucleases that act specifically on single stranded nucleic acid. For example, in certain embodiments, one may employ a ligation probe set or sets that include a protective group on one end such that, when the ligation probes are ligated to one another, both ends of the ligation product will be protected from exonuclease digestion. In such embodiments, unligated probes are not protected on one end such that unligated probes are digested by exonuclease. In certain such embodiments, the 5′ end of the first probe includes a protective group, and the 3′ end of the second probe includes a protective group. One skilled in the art will appreciate certain exonucleases and certain protective groups that may be employed according to certain embodiments. In certain embodiments, biotin is used as a protective group. In certain embodiments, one may employ a method such that the exonuclease activity is substantially removed prior to an amplification reaction. In certain embodiments, one may employ an exonuclease that loses activity when exposed to a particular temperature for a given amount of time.

Amplification according to various embodiments encompasses a broad range of techniques for amplifying nucleic acid sequences, either linearly or exponentially. Exemplary amplification techniques include, but are not limited to, PCR or any other method employing a primer extension step. Other nonlimiting examples of amplification are ligase detection reaction (LDR), and ligase chain reaction (LCR). Another nonlimiting exemplary amplification is a whole genome amplification. Amplification methods may comprise thermal-cycling or may be performed isothermally. The term “amplification product” includes products from any number of cycles of amplification reactions and primer extension reactions unless otherwise apparent from the context.

In certain embodiments, amplification methods comprise at least one cycle of amplification, for example, but not limited to, the sequential procedures of: hybridizing primers to primer-specific portions (or complements of primer-specific portions) of the ligation product or amplification products from any number of cycles of an amplification reaction; synthesizing a strand of nucleotides in a template-dependent manner using a polymerase; and denaturing the newly-formed nucleic acid duplex to separate the strands. The cycle may or may not be repeated.

Descriptions of certain amplification techniques can be found, among other places, in H. Ehrlich et al., Science, 252:1643-50 (1991); M. Innis et al., PCR Protocols: A Guide to Methods and Applications, Academic Press, New York, N.Y. (1990); R. Favis et al., Nature Biotechnology 18:561-64 (2000); H. F. Rabenau et al., Infection 28:97-102 (2000); Sambrook and Russell; and Ausubel et al.

Primer extension according to the present teachings is an amplification process comprising elongating a primer that is annealed to a template in the 5′ to 3′ direction using a template-dependent polymerase. According to certain embodiments, with appropriate buffers, salts, pH, temperature, and nucleotide triphosphates, including analogs and derivatives thereof, a template dependent polymerase incorporates nucleotides complementary to the template strand starting at the 3′-end of an annealed primer, to generate a complementary strand. Detailed descriptions of primer extension according to certain embodiments can be found, among other places in Sambrook et al., Sambrook and Russell, and Ausubel et al.

Certain embodiments of amplification may employ multiplex amplification, in which multiple target sequences are simultaneously amplified (see, e.g., H. Geada et al., Forensic Sci. Int. 108:31-37 (2000) and D. G. Wang et al., Science 280:1077-82 (1998)).

In certain embodiments, one employs asymmetric PCR. According to certain embodiments, asymmetric PCR comprises an amplification reaction composition comprising (i) at least one primer set in which there is an excess of one primer (relative to the other primer in the primer set); (ii) at least one primer set that comprises only a first primer or only a second primer; (iii) at least one primer set that, during given amplification conditions, comprises a primer that results in amplification of one strand and comprises another primer that is disabled; or (iv) at least one primer set that meets the description of both (i) and (iii) above. Consequently, when the ligation product is amplified, an excess of one strand of the amplification product (relative to its complement) is generated.

In certain embodiments, one may use at least one primer set wherein the melting temperature (Tm₅₀) of one of the primers is higher than the Tm₅₀ of the other primer. Such embodiments have been called asynchronous PCR (A-PCR). See, e.g., published U.S. Patent Application No. US 2003-0207266 A1, filed Jun. 5, 2001. In certain embodiments, the Tm₅₀ of the first primer is at least 4-15° C. different from the Tm₅₀ of the second primer. In certain embodiments, the Tm₅₀ of the first primer is at least 8-15° C. different from the Tm₅₀ of the second primer. In certain embodiments, the Tm₅₀ of the first primer is at least 10-15° C. different from the Tm₅₀ of the second primer. In certain embodiments, the Tm₅₀ of the first primer is at least 10-12° C. different from the Tm₅₀ of the second primer. In certain embodiments, in at least one primer set, the Tm₅₀ of the at least one first primer differs from the melting temperature of the at least one second primer by at least about 4° C., by at least about 8° C., by at least about 10° C., or by at least about 12° C.

In certain embodiments of A-PCR, in addition to the difference in Tm₅₀ of the primers in a primer set, there is also an excess of one primer relative to the other primer in the primer set. In certain embodiments, there is a five to twenty-fold excess of one primer relative to the other primer in the primer set. In certain embodiments of A-PCR, the primer concentration is at least 50 nM.

In A-PCR according to certain embodiments, one may use conventional PCR in the first cycles such that both primers anneal and both strands are amplified. By raising the temperature in subsequent cycles, however, one may disable the primer with the lower Tₘ such that only one strand is amplified. Thus, the subsequent cycles of A-PCR in which the primer with the lower Tₘ is disabled result in asymmetric amplification. Consequently, when the ligation product is amplified, an excess of one strand of the amplification product (relative to its complement) is generated.

According to certain embodiments of A-PCR, the level of amplification can be controlled by changing the number of cycles during the first phase of conventional PCR cycling. In such embodiments, by changing the number of initial conventional cycles, one may vary the amount of the double strands that are subjected to the subsequent cycles of PCR at the higher temperature in which the primer with the lower Tₘ is disabled.

In certain embodiments, an A-PCR protocol may comprise use of a pair of primers, each of which has a concentration of at least 50 nM. In certain embodiments, conventional PCR, in which both primers result in amplification, is performed for the first 20-30 cycles. In certain embodiments, after 20-30 cycles of conventional PCR, the annealing temperature increases to 66-70° C., and PCR is performed for 5 to 40 cycles at the higher annealing temperature. In such embodiments, the lower Tₘ primer is disabled during such 5 to 40 cycles at the higher annealing temperature. In such embodiments, asymmetric amplification occurs during the second phase of PCR cycles at a higher annealing temperature.

Certain methods of optimizing amplification reactions are known to those skilled in the art. For example, it is well known that PCR may be optimized by altering times and temperatures for annealing, polymerization, and denaturing, as well as changing the buffers, salts, and other reagents in the reaction composition. Optimization may also be affected by the design of the amplification primers used. For example, the length of the primers, as well as the G-C:A-T ratio may alter the efficiency of primer annealing, thus altering the amplification reaction. See, e.g., James G. Wetmur, “Nucleic Acid Hybrids, Formation and Structure,” in Molecular Biology and Biotechnology, pp. 605-8, (Robert A. Meyers ed., 1995).

In certain embodiments, one may subject the initial sample to an amplification reaction to increase the amount of target nucleic acid to which probes in a ligation probe set will hybridize.

In certain embodiments, different addressable portions are used to determine the probes that have been ligated. In certain embodiments, the addressable portions (and/or their complements) hybridize to particular capture oligonucleotides on a support.

In certain embodiments, different ligation and/or amplification products are detected by mobility discrimination using separation techniques. In certain embodiments, the addressable portions (and/or their complements) may have uniquely identifiable lengths or molecular weights. In certain embodiments, the addressable portion that corresponds to one target nucleic acid sequence is 2 nucleotides in length, the addressable portion that corresponds to a second target nucleic acid sequence is 4 nucleotides in length, the addressable portion that corresponds to a third target nucleic acid sequence is 6 nucleotides in length, and so forth. In certain embodiments the addressable portion is less than 101 nucleotides (i.e., 0 to 100 nucleotides) long, less than 41 nucleotides (i.e., 0 to 40 nucleotides) long, or 2 to 36 nucleotides long. In certain embodiments, the addressable portions that correspond to a particular target nucleic acid sequence will differ in length from the addressable portions that correspond to different target nucleic acid sequences by at least two nucleotides.

In certain embodiments, an addressable portion (and/or its complement) may comprise a sequence that is complementary to at least a portion of a particular mobility-modifier. In certain embodiments, a mobility modifier comprises (1) a complementary addressable portion (or an addressable portion) for selectively hybridizing to the addressable portion (or complementary addressable portion) of a ligation product and/or an amplification product, and (2) a tail portion for effecting a particular mobility in a mobility-dependent analysis technique (MDAT), e.g., electrophoresis, e.g., U.S. Pat. Nos. 6,395,486 and 6,734,296. Thus, in certain embodiments, ligation products and/or amplification products can be separated by molecular weight or length to determine the products present.

In certain embodiments, the detection of a ligation product and/or an amplification product in a particular molecular weight or length bin indicates the presence of the corresponding target nucleic acid sequence in the sample.

In certain embodiments, each different ligation product and/or amplification product comprises a different addressable portion. In certain such embodiments, the detection of one or more ligation products and/or amplification products includes capturing the ligation product and/or amplification product on a solid support. In certain such embodiments, the detection of one or more ligation products and/or amplification products further includes, combining the one or more ligation products and/or amplification products with at least two different sequence-specific mobility-modifiers, wherein each different mobility-modifier is capable of sequence-specific binding to a different addressable portion and comprises (a) a tag complement for specifically binding the addressable portion of one of the one or more ligation products and/or amplification products, and (b) a tail which imparts to each mobility modifier a mobility that is distinctive relative to the mobilities of one or more other of said at least two different mobility-modifiers in a mobility-dependent analysis technique. In certain such embodiments, the detection of one or more ligation products and/or amplification products further includes removing mobility-modifiers that are not sequence-specifically bound to the one or more ligation products and/or amplification products from mobility-modifiers that are sequence-specifically bound to the one or more ligation products and/or amplification products. In certain such embodiments, the detection of one or more ligation products and/or amplification products further includes releasing the sequence-specifically bound mobility-modifiers from the one or more ligation products and/or amplification products. In certain such embodiments, the detection of one or more ligation products and/or amplification products further includes subjecting the released mobility-modifiers to a mobility-dependent analysis technique. In certain such embodiments, the detection of one or more ligation products and/or amplification products further includes detecting the one or more ligation products and/or amplification products by detecting distinctive positions of the mobility-modifiers.

Descriptions of exemplary, but nonlimiting, MDATs may be found, among other places, in U.S. Pat. Nos. 5,470,705, 5,514,543, 5,580,732, 5,624,800, and 5,807,682.

In certain exemplary embodiments, air-dried ligation product and/or amplification product pellets, comprising ligation products and/or amplification products of uniquely identifiable molecular weight, are resuspended in buffer or deionized formamide. In certain embodiments, the resuspended samples and a molecular weight marker (e.g., GS 500 size standard, Applied Biosystems, Foster City, Calif.) are loaded onto an electrophoresis platform (e.g., ABI Prism™ Genetic Analyzer, Applied Biosystems) and electrophoresed in a capillary comprising an appropriate polymer, such as POP-4 polymer (Applied Biosystems). In certain embodiments, the bands are detected or quantitated, and their position relative to the marker is determined. In certain embodiments, the bands are identified based on their relative electrophoretic mobility, indicating the presence of their respective target nucleic acid sequence in the sample. In certain embodiments, the bands may be quantitated, for example, based on the relative intensity of the associated label.

According to certain embodiments, certain addressable portions and complementary addressable portions should form a complex that (1) is stable under conditions typically used in nucleic acid analysis methods, e.g., aqueous, buffered solutions at room temperature; (2) is stable under mild nucleic-acid denaturing conditions; and (3) does not adversely effect the sequence specific binding of a target-specific portion of a probe with a target nucleic acid sequence. In certain embodiments, addressable portions and complementary addressable portions accommodate sets of distinguishable addressable portions and complementary addressable portions such that a plurality of different ligation products and/or amplification products and associated mobility modifiers may be present in the same reaction volume without causing unintended cross-interactions among the addressable portions, complementary addressable portions, target nucleic acid sequence, and target-specific portions of the probes. Certain methods for selecting sets of addressable portions that minimally cross hybridize are described, e.g., in Brenner and Albrecht, PCT Patent Application No. WO 96/41011.

In certain embodiments, the addressable portions and complementary addressable portions each comprise polynucleotides. In certain embodiments, the polynucleotide complementary addressable portions are rendered non-extendable by a polymerase, e.g., by including sugar modifications such as a 3′-phosphate, a 3′-acetyl, a 2′-3′-dideoxy, a 3′-amino, and a 2′-3′ dehydro.

In certain embodiments, an addressable portion and complementary addressable portion pair comprises an addressable portion that is a conventional synthetic polynucleotide, and a complementary addressable portion that is PNA. In certain embodiments, where the PNA complementary addressable portion has been designed to form a triplex structure with a tag, the complementary addressable portion may include a “hinge” region in order to facilitate triplex binding between the addressable portion and complementary addressable portion. In certain embodiments, addressable portions and complementary addressable portion sequences comprise repeating sequences. Such repeating sequences in the addressable portions and complementary addressable portion are used in certain embodiments for their (1) high binding affinity, (2) high binding specificity, and (3) high solubility. An exemplary repeating sequence for use as a duplex-forming addressable portion or complementary addressable portion in certain embodiments is (CAG)ₙ, where the three base sequence is repeated from about 1 to 10 times (see, e.g., Boffa, et al., PNAS (USA), 92:1901-05 (1995); Wittung, et al., Biochemistry, 36:7973-79 (1997)). An exemplary repeating sequence for use as a triplex-forming addressable portion or complementary addressable portion in certain embodiments is (TCC)ₙ.

PNA and PNA/DNA chimera molecules can be synthesized using well known methods on commercially available, automated synthesizers, with commercially available reagents (see, e.g., Dueholm, et al., J. Org. Chem., 59:5767-73 (1994); Vinayak, et al., Nucleosides & Nucleotides, 16:1653-56 (1997)).

In certain embodiments, the tail portion of a mobility modifier may be any entity capable of effecting a particular mobility of a mobility modifier or of a complex comprising the mobility modifier, such as all or a portion of a ligation product and/or an amplification product associated with the mobility modifier, in a mobility-dependent analysis technique. In certain embodiments, the tail portion of a mobility modifier may be any entity capable of effecting a particular mobility of the mobility modifier. In certain embodiments, a tail portion of the mobility modifier may have one or more of the following characteristics: (1) have a low polydispersity in order to effect a well-defined and easily resolved mobility, e.g., Mw/Mn less than 1.05; (2) be soluble in an aqueous medium; (3) not adversely affect probe-target hybridization or addressable portion/complementary addressable portion hybridization; and (4) be available in sets such that members of different sets impart distinguishable mobilities.

In certain embodiments, the tail portion of the mobility modifier comprises a polymer. In certain embodiments, the polymer may be a homopolymer, random copolymer, or block copolymer. In certain embodiments, the polymer may have a linear, comb, branched, or dendritic architecture. In certain embodiments, mobility modifiers comprise more than one polymer chain element, where the elements collectively form a tail portion.

Exemplary polymers include, but are not limited to, hydrophilic, or at least sufficiently hydrophilic when bound to a complementary addressable portion (or addressable portion) so that the complementary addressable portion (or addressable portion) is readily soluble in aqueous medium. In certain embodiments, where the mobility-dependent analysis technique is electrophoresis, the polymers are uncharged or have a charge/subunit density that is substantially less than that of the amplification product.

In certain embodiments, the polymer is polyethylene oxide (PEO), e.g., formed from one or more hexaethylene oxide (HEO) units, where the HEO units are joined end-to-end to form an unbroken chain of ethylene oxide subunits. Certain embodiments include, but are not limited to, a chain composed of N 12mer PEO units, and a chain composed of N tetrapeptide units, where N is an adjustable integer (e.g., Grossman et al., U.S. Pat. No. 5,777,096).

In certain embodiments, coupling of the polymer tails to a polynucleotide complementary addressable portion (or addressable portion) can be carried out by an extension of conventional phosphoramidite polynucleotide synthesis methods, or by other standard coupling methods, e.g., a bis-urethane tolyl-linked polymer chain may be linked to a polynucleotide on a solid support via a phosphoramidite coupling. In certain embodiments, a polymer chain can be built up on a polynucleotide by stepwise addition of polymer-chain units to the polynucleotide, e.g., using standard solid-phase polymer synthesis methods.

In certain embodiments, the contribution of the tail to the mobility of the mobility modifier, the ligation product mobility modifier complex and/or amplification product mobility modifier complex, generally depends on the size of the tail. In certain embodiments, addition of charged groups to the tail, e.g., charged linking groups in the PEO chain, or charged amino acids in a polypeptide chain, may be used to achieve selected mobility characteristics. In certain embodiments, the mobility of a complex may be influenced by the properties of the ligation product and/or amplification product, e.g., in electrophoresis in a sieving medium, a larger probe in certain embodiments, may reduce the electrophoretic mobility of the complex comprising a mobility modifier.

When a complementary addressable portion (or addressable portion) is a polynucleotide, the complementary addressable portion (or addressable portion) may comprise all, part, or none of the tail portion of the mobility modifier. In certain embodiments, the complementary addressable portion (or addressable portion) may consist of some or all of the tail portion of the mobility modifier. In certain embodiments, the complementary addressable portion (or addressable portion) does not comprise any portion of the tail portion of the mobility modifier. For example, in certain embodiments, because PNA is uncharged, particularly when using free solution electrophoresis as the mobility-dependent analysis technique, the same PNA oligomer may act as both a complementary addressable portion (or addressable portion) and a tail portion of a mobility modifier.

In certain embodiments, the complementary addressable portion (or addressable portion) includes a hybridization enhancer, where, as used herein, the term “hybridization enhancer” means a moiety that serves to enhance, stabilize, or otherwise positively influence hybridization between two polynucleotides. Certain exemplary embodiments include, but are not limited to, intercalators (e.g., U.S. Pat. No. 4,835,263), minor-groove binders (e.g., U.S. Pat. No. 5,801,155), and cross-linking functional groups. In various embodiments, the hybridization enhancer may be attached to any portion of a mobility modifier. In certain embodiments, the hybridization enhancer is covalently attached to a mobility modifier. In certain embodiments, a hybridization enhancer is a minor-groove binder, e.g., but not limited to, netropsin, distamycin, and the like.

In certain embodiments, a plurality of mobility modifiers, ligation product/mobility modifier complexes, and/or amplification product/mobility modifier complexes are resolved via a MDAT.

In certain embodiments, mobility modifiers, ligation product/mobility modifier complexes, and/or amplification product/mobility modifier complexes are resolved (separated) by liquid chromatography. Exemplary stationary phase media for use in certain exemplary methods include reversed-phase media (e.g., C-18 or C-8 solid phases), ion-exchange media (particularly anion-exchange media), and hydrophobic interaction media. In certain embodiments, the ligation product/mobility modifier complexes and/or amplification product/mobility modifier complexes are separated by micellar electrokinetic capillary chromatography (MECC).

In certain embodiments, reversed-phase chromatography is carried out using an isocratic, or a linear, curved, or stepped solvent gradient, wherein the level of a nonpolar solvent such as acetonitrile or isopropanol in aqueous solvent is increased during a chromatographic run, causing analytes to elute sequentially according to affinity of each analyte for the solid phase. In certain embodiments, for separating polynucleotides, an ion-pairing agent (e.g., a tetra-alkylammonium) is included in the solvent to mask the charge of phosphate.

According to certain embodiments, the mobility modifier, ligation product/mobility modifier complexes, and/or amplification product/mobility modifier complexes are resolved by electrophoresis in a sieving or non-sieving matrix and quantitated. In certain embodiments, the electrophoretic separation is carried out in a capillary tube by capillary electrophoresis (see, e.g., Capillary Electrophoresis: Theory and Practice, Grossman and Colburn eds., Academic Press (1992)). Sieving matrices that may be used include, but are not limited to, covalently crosslinked matrices, such as polyacrylamide covalently crosslinked with bis-acrylamide; gel matrices formed with linear polymers (e.g., Madabhushi et al., U.S. Pat. No. 5,552,028); and gel-free sieving media (e.g., Grossman et al., U.S. Pat. No. 5,624,800; Hubert and Slater, Electrophoresis, 16: 2137-2142 (1995); Mayer et al., Analytical Chemistry, 66(10): 1777-1780 (1994)). In certain embodiments, the electrophoresis medium may contain a nucleic acid denaturant, such as 7M formamide, for maintaining polynucleotides in single-stranded form. Certain suitable capillary electrophoresis instrumentation are commercially available, e.g., the ABI PRISM™ Genetic Analyzer (Applied Biosystems).

In certain embodiments, following at least one amplification cycle, the amplification products are separated based on their molecular weight or length or mobility by, for example, without limitation, gel electrophoresis, HPLC, MALDI-TOF, gel filtration, or mass spectroscopy. In certain embodiments, the detection and quantitation of a labeled sequence at a particular mobility address indicates that the sample or starting material contains the corresponding target nucleic acid sequence.

In certain embodiments, ligation products may be detected by hybridization of addressable portions (or complementary addressable portions) of the ligation products to capture oligonucleotides on an addressable support. In certain embodiments, one may determine the methylation state of a target nucleotide in view of the label. In certain embodiments, the addressable portion (or complementary addressable portion) may be a mobility modifier, which allows separation of ligation products with different addressable portions by a mobility dependent analysis technique. In certain embodiments, the addressable portions may be hybridized to appropriate mobility modifiers and then the presence of particular ligation products may be detected using an MDAT.

In certain embodiments, addressable portions (or complementary addressable portions) interact with particular beads that comprise complementary addressable portions (or addressable portions). See e.g., U.S. Patent Application Publication No. US 2003-0165935 A1.

In certain embodiments, addressable portions (or complementary addressable portions) interact with particular labeled probes that include complementary addressable portions (or addressable portions). See, e.g., U.S. Patent Application Publication No. US 2004-0121371 A1.

In certain embodiments, ligation products and/or amplification products are detected using a double-stranded dependent label, such as an intercalating dye, e.g., as disclosed in U.S. Patent Application Publication No. US 2004-0050828 A1.

Certain Exemplary Embodiments of Detecting Targets

In certain embodiments, methods, reagents, and kits are provided for determining the methylation state of a target nucleotide. In certain embodiments, one employs a ligation reaction that results in a given ligation product only if a particular target nucleic acid sequence comprising a target nucleotide having a particular methylation state is present in a sample. In certain embodiments, ligation products may form even if the appropriate target nucleic acid sequence comprising the target nucleotide having the appropriate methylation state is not in the sample, but such ligation occurs to a measurably lesser extent than when the appropriate target nucleic acid sequence is in the sample. Exemplary ligation reactions, include, but are not limited to, those discussed in U.S. Pat. No. 6,027,889, Published PCT Patent Application No. WO 01/92579, published PCT Patent Application WO 97/31256, and U.S. patent application Ser. Nos. 09/584,905 and 10/011,993.

In certain embodiments, a ligation reaction composition comprises a blocking probe and ligation probe set. In certain embodiments, a ligation probe set is subjected to at least one cycle of ligation, wherein adjacently hybridized first and second probes are ligated together to form a ligation product only if the particular target nucleic-acid sequence comprising a target nucleotide having an appropriate methylation state is present in the sample. In certain such embodiments, the blocking probe hybridizes to a portion of the target nucleic acid sequence comprising the target nucleotide if the target nucleotide does not have the appropriate methylation state. In certain such embodiments, hybridization of the blocking probe to the portion of the target nucleic acid sequence blocks hybridization of the first probe, the second probe, or both the first probe and the second probe to the portion of the target nucleic acid sequence. In certain embodiments, ligation products may form even if the appropriate target nucleic acid sequence comprising the target nucleotide having the appropriate methylation state is not in the sample, but such ligation occurs to a measurably lesser extent than when the appropriate target nucleic acid sequence is in the sample.

In certain such embodiments, for each target nucleic acid sequence to be detected, one forms a ligation reaction composition comprising a blocking probe and a ligation probe set comprising at least one first probe and at least one second probe. In certain embodiments, probes of a ligation probe set hybridize to a target nucleic acid sequence comprising a methylated target nucleotide to a measurably lesser extent than the blocking probe. In certain such embodiments, the blocking probe hybridizes to a target nucleic acid sequence comprising an unmethylated target nucleotide to a measurably lesser extent than the probes of the ligation probe set.

In certain embodiments, probes of a ligation probe set hybridize to a target nucleic acid sequence comprising an unmethylated target nucleotide to a measurably lesser extent than the blocking probe. In certain such embodiments, the blocking probe hybridizes to a target nucleic acid sequence comprising a methylated target nucleotide to a measurably lesser extent than the probes of the ligation probe set.

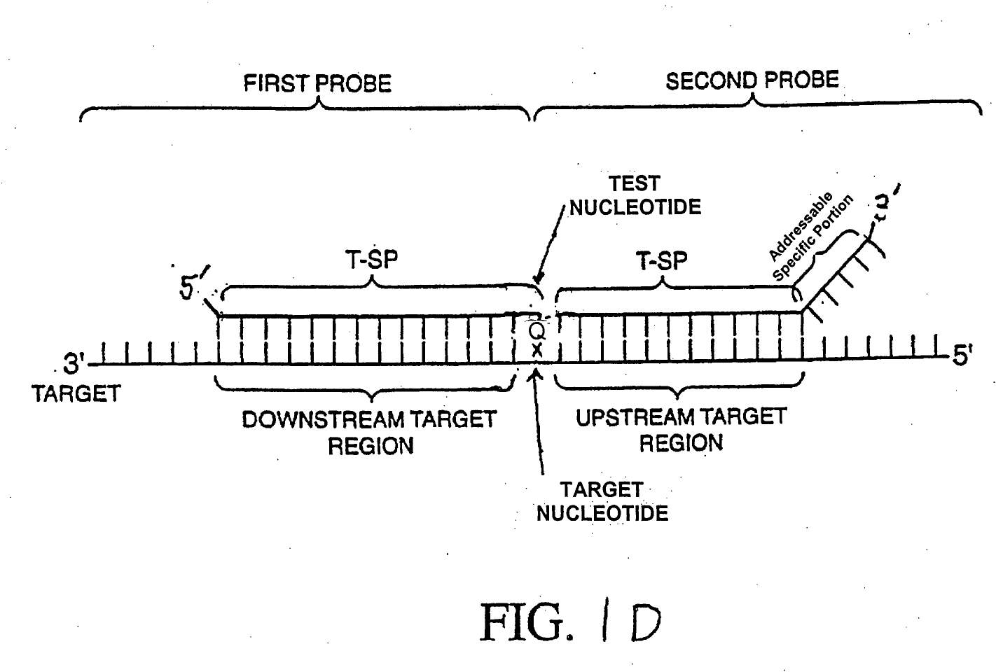

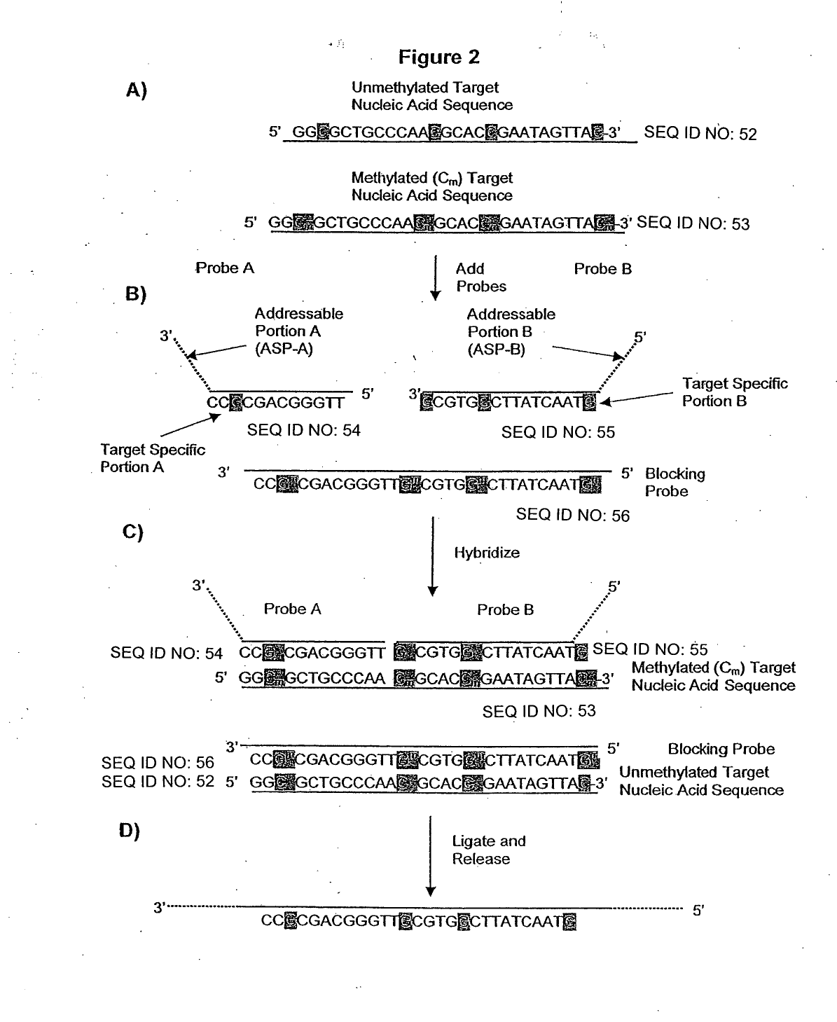

In certain embodiments, a blocking probe comprises at least one modified nucleotide. In certain such embodiments, the at least one modified nucleotide forms a base pair with the methylated nucleotide but does not form a base pair with the unmethylated nucleotide. In certain other embodiments, the modified nucleotide forms a base pair with the unmethylated nucleotide but does not form a base pair with the methylated nucleotide. In certain embodiments, the modified nucleotide is a modified guanine, which forms a base pair with methylated cytosine but will not base pair with unmethylated cytosine. In certain other embodiments, the modified nucleotide is a modified guanine which will not base pair with a methylated cytosine due to steric interference but will base pair with unmethylated cytosine, see, e.g., FIG. 2(C).

In certain embodiments, a selective ligase is also included in a ligation reaction composition. In certain embodiments, such a ligase results in selective ligation of adjacently hybridized probes of a ligation probe set if the target nucleotide is methylated. In certain embodiments, such a ligase results in selective ligation of adjacently hybridized probes of a ligation probe set if the target nucleotide is not methylated. The term “selective ligation” means that ligation occurs to a measurably lesser extent in the presence of target nucleic acid sequences that do not have a target nucleotide in the appropriate methylation state than in the presence of target nucleic acid sequences that do have a target nucleotide with the appropriate methylation state. In certain embodiments, ligation only occurs in the presence of target nucleic acid sequences that have a target nucleotide in the appropriate methylation state.

Certain Exemplary Embodiments Comprising Modifying Target Nucleic Acid Sequences

In certain embodiments, the sample is first incubated with a modifying agent that selectively modifies the target nucleotide, depending on the methylation state of the target nucleotide. The term “selectively modifies” means that modification of target-nucleotides occurs to a measurably lesser extent with target nucleotides that do not have the appropriate methylation state than with target nucleotides that have the appropriate methylation state. In certain embodiments, modification only occurs with target nucleic acid sequences that have a target nucleotide in the appropriate methylation state. In certain embodiments, a modifying agent selectively binds to methylated nucleotides. In certain other embodiments, a modifying agent selectively binds to unmethylated nucleotides. The term “selectively binds” means that binding of target nucleotides occurs to a measurably lesser extent with target nucleotides that do not have the appropriate methylation state than with target nucleotides that have the appropriate methylation state. In certain embodiments, binding only occurs in the presence of target nucleic acid sequences that have a target nucleotide in the appropriate methylation state. In certain embodiments, the ligation reaction is affected by the presence or absence of a bound modifying agent. For example, in certain embodiments, a blocking probe selectively binds to a target nucleic acid sequence having bound modifying agent and a ligation probe set selectively binds to a target nucleic acid sequence that does not have bound modifying agent. In certain such embodiments, ligation occurs to a measurably lesser extent if a target nucleic acid sequence having bound modifying agent is present. In certain such embodiments, ligation only occurs if a target nucleic acid sequence having bound modifying agent is present. In certain embodiments, a blocking probe selectively binds to a target nucleic acid sequence that does not have bound modifying agent and a ligation probe set selectively binds to a target nucleic acid sequence having bound modifying agent. In certain such embodiments, ligation occurs to a measurably lesser extent if a target nucleic acid sequence having bound modifying agent is absent. In certain such embodiments, ligation only occurs if a target nucleic acid sequence having bound modifying agent is absent.

In certain embodiments, the modifying agent selectively, chemically alters a target nucleotide, depending on the methylation state of the target nucleotide. The term “selectively, chemically alters” means that chemical alteration of target nucleotides occurs to a measurably lesser extent with target nucleotides that do not have the appropriate methylation state than with target nucleotides that have the appropriate methylation state. In certain embodiments, chemical alteration only occurs in the presence of target nucleic acid sequences that have a target nucleotide in the appropriate methylation state. In certain embodiments, a modifying agent selectively, chemically alters methylated nucleotides. In certain other embodiments, a modifying agent selectively, chemically alters unmethylated nucleotides. In certain embodiments, the ligation reaction is affected by the presence or absence of a chemically altered nucleotide. In certain embodiments, a blocking probe selectively binds to a target nucleic acid sequence comprising at least one chemically altered nucleotide and a ligation probe set selectively binds to a target nucleic acid sequence that does not comprise at least one chemically altered nucleotide. In certain such embodiments, ligation occurs to a measurably lesser extent if a target nucleic acid sequence comprising at least one chemically altered nucleotide is present. In certain such embodiments, ligation only occurs if a target nucleic acid sequence comprising at least one chemically altered nucleotide is present. In certain embodiments, a blocking probe selectively binds to a target nucleic acid sequence that does not comprise at least one chemically altered nucleotide and a ligation probe set selectively binds to a target nucleic acid sequence comprising at least one chemically altered nucleotide. In certain such embodiments, ligation occurs to a measurably lesser extent if a target nucleic acid sequence comprising at least one chemically altered nucleotide is absent. In certain such embodiments, ligation only occurs if a target nucleic acid sequence comprising at least one chemically altered nucleotide is absent.

In certain embodiments, the modifying agent selectively converts a target nucleotide to a converted nucleotide, depending on the methylation state of the target nucleotide. The term “selectively converts” means that conversion of target nucleotides occurs to a measurably lesser extent with target nucleotides that do not have the appropriate methylation state than with target nucleotides that have the appropriate methylation state. In certain embodiments, conversion only occurs in the presence of target nucleic acid sequences that have a target nucleotide in the appropriate methylation state. In certain embodiments, a modifying agent selectively converts methylated nucleotides to converted nucleotides. In certain embodiments, a modifying agent selectively converts unmethylated nucleotides to converted nucleotides. In certain embodiments, at least one ligation probe set may be used to differentiate between targets with unconverted nucleotides and targets with converted nucleotides.

In certain embodiments, bisulfite is employed as a modifying agent. See, e.g., U.S. Pat. No. 6,265,171; U.S. Pat. No. 6,331,393; Boyd and Zon, Anal. Biochem. 326: 278-280, 2004; U.S. Provisional Patent Application Ser. Nos. 60/499,113; 60/520,942; 60/499,106; 60/523,054; 60/498,996; 60/520,941; 60/499,082; and 60/523,056. Incubating target nucleic acid sequence with bisulfite results in deamination of a substantial portion of unmethylated cytosines, which converts such cytosines to uracil. Methylated cytosines are deaminated to a measurably lesser extent. In certain embodiments, the sample is then amplified or replicated, resulting in the uracil bases being replaced with thymine. Thus, in certain embodiments, a substantial portion of unmethylated target cytosines ultimately become thymines, while a substantial portion of methylated cytosines remain cytosines. In certain embodiments, the identity of the nucleotide (cytosine, uracil, or thymine) of the target may be determined by a ligation assay. In certain embodiments, the identity of the nucleotide (cytosine, uracil, or thymine) of the target may be determined by a ligation and amplification assay.

In certain embodiments, other modifying agents may be used. In certain embodiments, the modifying agent need not catalyze deamination reactions and the converted nucleotide need not be uracil or thymine. Certain embodiments may employ any agent that is capable of selectively converting either methylated target nucleotides or unmethylated target nucleotides to another nucleotide.

As discussed above, certain embodiments employ a modifying agent that selectively converts either methylated or unmethylated target nucleotides of a target nucleic acid sequence to a converted nucleotide. In certain such embodiments, after incubation with the modifying agent, the target nucleic acid sequence that has been incubated is called the test target nucleic acid sequence. In certain embodiments, the nucleotide of a probe that hybridizes to the target nucleotide is called the test nucleotide. In certain such embodiments, one forms a test composition comprising at least one test target nucleic acid sequence by incubating the at least one target nucleic acid sequence with the modifying agent to selectively convert either one or more methylated nucleotides or one or more unmethylated nucleotides to converted nucleotides.

In certain embodiments, one forms a ligation reaction composition comprising the test composition, a blocking probe, and a ligation probe set for each target nucleic acid sequence. In certain embodiments, the ligation probe set comprises at least one first probe comprising a first target-specific portion, and at least one second probe comprising a second target-specific portion. In certain embodiments, at least one of the at least one first probe and the at least one second probe comprises at least one test nucleotide. In certain embodiments, the test nucleotide is complementary to the target nucleotide and a nucleotide of the blocking probe forms a base pair with the converted nucleotide. In certain embodiments, the test nucleotide is complementary to the converted nucleotide and a nucleotide of the blocking probe forms a base pair with the target nucleotide.

In certain embodiments, at least one of the at least one first probe and the at least one second probe comprises a label. In certain embodiments, a probe comprising a label further comprises a test nucleotide. In certain embodiments, a probe comprising a label further comprises a test nucleotide that is complementary to a target nucleotide. In certain embodiments, such a probe may be used to detect the presence or absence of a target nucleotide that has not been converted. In certain embodiments, a probe comprising a label further comprises a test nucleotide that is complementary to the converted nucleotide. In certain embodiments, such a probe may be used to detect the presence or absence of a target nucleotide that has been converted.

In certain embodiments, at least one of the at least one first probes comprises a label and a test nucleotide that is complementary to a target nucleotide; and at least one of the at least one first probes comprises a different label and a test nucleotide that is complementary to the converted nucleotide. In certain such embodiments, the different labels provide detectably different signals.

In certain embodiments, the reaction composition comprises a blocking probe and at least one of the at least one first probes comprises a label and at least one of the at least one second probes comprises a test nucleotide. In certain such embodiments, the second probe comprises a test nucleotide that is complementary to the target nucleotide and a nucleotide of the blocking probe forms a base pair to the converted nucleotide. In certain other embodiments, the second probe comprises a test nucleotide that is complementary to the converted nucleotide and a nucleotide of the blocking probe forms a base pair with the target nucleotide. In certain such embodiments, the ligation reaction composition is subjected to at least one cycle of ligation. In certain embodiments, after at least one cycle of ligation, the methylation state of the target nucleotide may be determined by detecting the presence or absence of ligation product comprising the label by separating ligation product from unligated probes based on size difference between the ligation product and unligated probe.

In certain embodiments, at least one probe comprises an addressable portion. In certain embodiments, addressable portions may be used in various combinations with labels to determine the methylation state of one or more target nucleotides at one or more loci. One skilled in the art will readily understand that one or more different addressable portions, labels, and test nucleotides may be used in various combinations on the same and/or on different first probes and/or on the same and/or different second probes.

In certain embodiments, either a methylated nucleotide is converted to a converted nucleotide, or an unmethylated nucleotide is converted to a converted nucleotide. In certain such embodiments, at least one of the at least one first probes comprises an addressable portion corresponding to a target nucleotide at one locus; and at least one other of the at least one first probes comprises an addressable portion corresponding to a different target nucleotide at a different locus. In certain embodiments, at least one of the at least one second probes comprises (1) a test nucleotide that is complementary to a target nucleotide, and (2) a label. In certain embodiments, a nucleotide of the blocking probe forms a base pair with the converted nucleotide. In certain embodiments, a nucleotide of the blocking probe forms a base pair with the target nucleotide. In certain embodiments, after at least one cycle of ligation, the addressable portions and the labels may be used to detect the presence or absence of ligation products to determine the methylation states of target nucleotides at the different loci.

In certain embodiments, either a methylated nucleotide is converted to a converted nucleotide, or an unmethylated nucleotide is converted to a converted nucleotide. In certain such embodiments, at least one of the at least one first probes comprises (1) an addressable portion specific for a target nucleotide at one locus, and (2) a test nucleotide that is complementary to target nucleotide. In certain such embodiments, a nucleotide of a first blocking probe forms a base pair with a converted nucleotide at the first locus. In certain such embodiments, at least one other of the at least one first probes comprises (1) an addressable portion specific for a different target nucleotide at a different locus and (2) a test nucleotide that is complementary to target nucleotide. In certain such embodiments, a nucleotide of a second blocking probe forms a base pair with a converted nucleotide at the second locus. In certain embodiments, at least one of the at least one second probes comprises a label. In certain embodiments, after at least one cycle of ligation, the addressable portions and the labels may be used to detect the presence or absence of ligation products to determine the methylation states of target nucleotides at the different loci.

In certain embodiments, either a methylated nucleotide is converted to a converted nucleotide, or an unmethylated nucleotide is converted to a converted nucleotide. In certain such embodiments, at least one of the at least one first probes comprises (1) an addressable portion specific for a target nucleotide at one locus, and (2) a test nucleotide that is complementary to the converted nucleotide. In certain such embodiments, a nucleotide of a first blocking probe forms a base pair with a target nucleotide at the first locus. In certain such embodiments, at least one other of the at least one first probes comprises (1) an addressable portion specific for a different target nucleotide at a different locus and (2) a test nucleotide that is complementary to the converted nucleotide. In certain such embodiments, a nucleotide of a second blocking probe forms a base pair with a target nucleotide at the second locus. In certain embodiments, at least one of the at least one second probes comprises a label. In certain embodiments, after at least one cycle of ligation, the addressable portions and the labels may be used to detect the presence or absence of ligation products to determine the methylation states of target nucleotides at the different loci.

An Exemplary Method

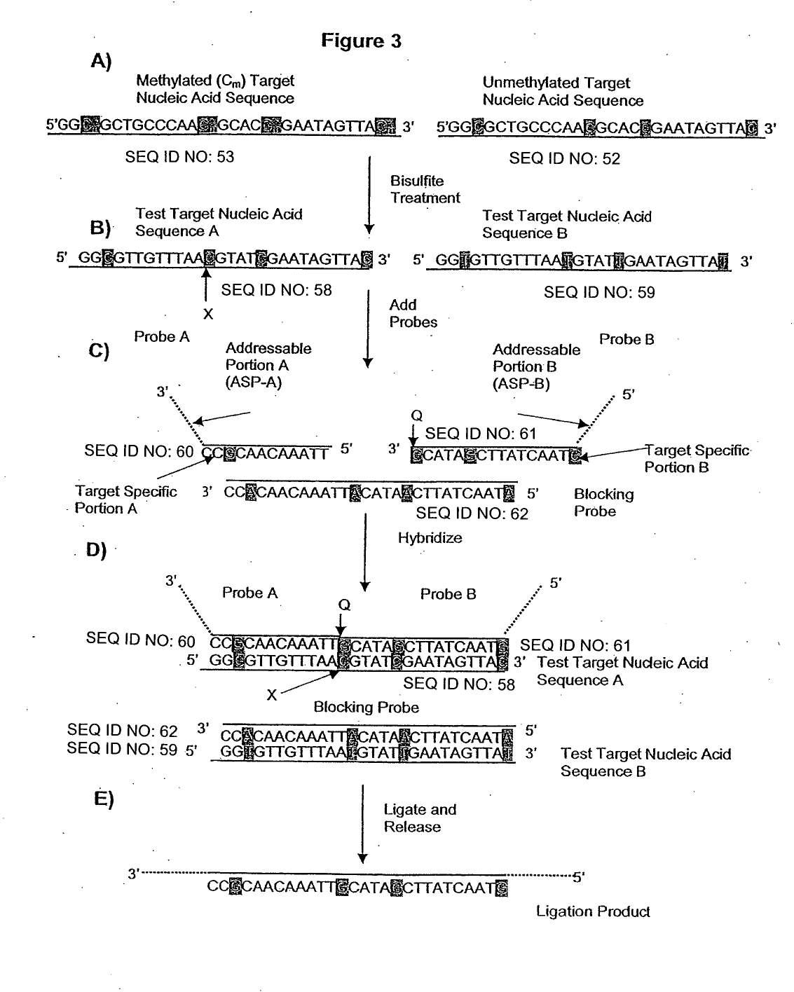

According to certain embodiments, Methylated (Cₘ) Target Nucleic Acid Sequence is exposed to bisulfite treatment to obtain Test Target Nucleic Acid Sequence A. In certain such embodiments, Unmethylated Target Nucleic Acid Sequence is exposed to bisulfite treatment to obtain Test Target Nucleic Acid Sequence B. In certain embodiments, the first and second probes in each ligation probe set are designed to be complementary to the sequences immediately flanking a target nucleotide (X) of a test target nucleic acid sequence (see, e.g., Probes A and B in FIGS. 3(C) and (D)). In the embodiment shown in FIG. 3, a first probe, Probe A, of a ligation probe set comprises an Addressable Portion A (ASP-A). In the embodiment shown in FIG. 3, the second probe, Probe B, of the ligation probe set comprises: (1) a test nucleotide (Q) that base pairs with the target nucleotide X; and (2) a different addressable portion, Addressable Portion B (ASP-B). In the embodiment shown in FIG. 3, the particular combination of ASP-A and ASP-B corresponds to the locus being analyzed.

In the embodiment shown in FIG. 3, Probe A and Probe B hybridize to Test Target Nucleic Acid Sequence A obtained after bisulfite treatment of a target nucleic acid sequence with methylated cytosine (see, e.g. FIGS. 3(A), (B), (C), and (D) showing Test Target Nucleic Acid Sequence A, Probe A, and Probe B). Although, Probe A and Probe B can also align to Test Target Nucleic Acid Sequence B, both Probe A and Probe B cannot do so without creating mismatches corresponding to the positions of the unmethylated target cytosines in Unmethylated Target Nucleic Acid Sequence (See, e.g., FIGS. 3(B) and (C) showing Test Target Nucleic Acid Sequence B, Probe A, and Probe B). In the embodiment shown in FIG. 3, when Probe A and Probe B hybridize to Test Target Nucleic Acid Sequence A, they align adjacent to each other such that they may be ligated together under the appropriate conditions (see, e.g., FIG. 3(D)).

The embodiment shown in FIG. 3, also shows a Blocking Probe which hybridizes to Test Target Nucleic Acid Sequence B. Although, the Blocking Probe can also align to Test Target Nucleic Acid Sequence A, it cannot do so without creating four mismatches corresponding to the positions of the methylated target cytosines in the Methylated (Cₘ) Target Nucleic Acid Sequence (See, e.g., FIGS. 3(B) and (C) showing Test Target Nucleic Acid Sequence A and the Blocking Probe). When the Blocking Probe in the embodiment shown in FIG. 3 hybridizes to Test Target Nucleic Acid Sequence B, it prevents Probe A and Probe B from binding to that test target nucleic acid. Thus, in the embodiment shown in FIG. 3, the Blocking Probe blocks hybridization of Probe A and Probe B to Test Target Nucleic Acid Sequence B.

In certain embodiments, if Probe A forms a ligation product with Probe B, one concludes that all four cytosines were methylated. In certain embodiments, if Probe A forms a ligation product with Probe B, one concludes that at least one of the four cytosines were methylated. In certain embodiments, if Probe A forms a ligation product with Probe B; one concludes that the cytosine that corresponds to the target nucleotide (X) was methylated.

Certain Multiplex Embodiments for Detecting the Methylation State of Target Nucleotides

In certain embodiments, multiplex methods for detecting the methylation state of target nucleotides comprise a blocking probe comprising modified nucleotides.

In certain embodiments, multiplex methods for detecting the methylation state of target nucleotides comprise modifying the target nucleic acid sequence.

In certain embodiments, multiple ligation probe sets and blocking probes may be used to determine the methylation state of multiple target nucleotides at multiple different loci. In certain such embodiments, one may employ multiple ligation probe sets that each include: different first probes that comprise different addressable portions and/or different second probes that comprise different addressable portions. In certain embodiments, the different addressable portions are used to separate ligation products for the different loci being analyzed.

In certain embodiments, the ligation reaction composition may comprise different ligation probe sets and different blocking probes for determining the methylation state of multiple different target nucleotides at multiple loci. In certain embodiments, one may, for example, without limitation, determine the methylation state of three different target nucleotides at three different loci in a sample (e.g., L1, L2, and L3) using three ligation probe sets and three blocking probes. See, e.g., Table 1 below.

TABLE 1

| Locus | Meth. State | Ligation Probe Set/Blocking Probe |
|---|---|---|
| L1 | 1 Meth | Probe A (red)--Probe Z (AP 1) |
| | 2 Unmeth | Blocking Probe BW |
| L2 | 1 Meth | Probe C (red)--Probe Y (AP 2) |
| | 2 Unmeth | Blocking Probe DV |
| L3 | 1 Meth | Probe E (red)--Probe X (AP 3) |
| | 2 Unmeth | Blocking Probe FU |

AP = Addressable Portion

In certain embodiments, one uses the three ligation probe sets described in Table 1 to determine the methylation state at the three loci L1, L2, and L3. In certain such embodiments, one determines the presence of methylation by the presence of the appropriate ligation product. For example, in certain embodiments, one determines that locus L1 is methylated if one detects AZ ligation product. In certain such embodiments, one infers that L1 is unmethylated if one does not detect AZ ligation product. In certain such embodiments, one determines that locus L2 is methylated if one detects CY ligation product. In certain such embodiments, one infers that L2 is unmethylated if one does not detect CY ligation product. In certain such embodiments, one determines that locus L3 is methylated if one detects EX ligation product. In certain such embodiments, one infers that L3 is unmethylated if one does not detect EX ligation product.

In certain embodiments, one may test a sample using two separate reaction compositions with two different groups of ligation probe sets and two different groups of blocking probes. For example, in certain embodiments, one may use a first ligation reaction composition comprising a portion of the sample and the ligation probe sets and blocking probes in Table 1 above. In certain such embodiments, one may also use a separate second ligation reaction composition comprising another portion of the sample and the ligation probe sets and blocking probes in Table 2 below.

TABLE 2

| Locus | Meth. State | Ligation Probe Set/Blocking Probe |
|---|---|---|
| L1 | 1 Meth | Blocking Probe AZ |
| | 2 Unmeth | Probe B (blue)--Probe W (AP 1) |
| L2 | 1 Meth | Blocking Probe CY |
| | 2 Unmeth | Probe D (blue)--Probe V (AP 2) |
| L3 | 1 Meth | Blocking Probe EX |
| | 2 Unmeth | F (blue)--Probe U (AP 3) |

AP = Addressable Portion

Thus, in certain embodiments, two different groups of ligation probe sets and two different groups of blocking probes are used in two separate reaction compositions to detect the methylation state of each target nucleotide at each locus. For example, and not limitation, in certain embodiments, the two first ligation probes of the two different ligation probe sets for each locus, for example, probes A and B for locus L1, comprise the same target-specific portion, but differ at the one or more test nucleotides. In certain such embodiments, the two second ligation probes of the two different ligation probe sets for each locus, for example, probes Z and W for locus L1, comprise the same target-specific portion, but differ at one or more test nucleotides.

Thus, in certain embodiments, such as the embodiments depicted in Tables 1 and 2, four probes A, B, Z, and W are used to form the two possible L1 ligation products. In such embodiments, an AZ ligation product indicates that locus L1 is methylated and a BW ligation product indicates that locus L1 is unmethylated. Likewise, probes C, D, V, and Y, are used to form the two possible L2 ligation products. Likewise, probes E, F, U, and X, are used to form the two possible L3 ligation products.

After ligation of adjacently hybridized first and second ligation probes, in certain embodiments, one can detect the presence or absence of a ligation product for each methylation state of a target nucleotide for each of the loci by using unique combinations of labels and addressable portions. For example, in certain embodiments, one may determine the methylation state of a target nucleotide by the label or labels detected at position 1 in view of addressable portion 1, determine the methylation state of a target nucleotide by the label or labels detected at position 2 in view of addressable portion 2, and determine the methylation state of a target nucleotide by the label or labels detected at position 3 in view of addressable portion 3.

For example and not limitation, in certain embodiments, the probes Z and W comprise the same addressable portion. In certain embodiments, the addressable portion of the second ligation probes is the same for all second ligation probes directed to the same locus, but is different for each different locus. Thus, in certain embodiments, each different addressable portion may be used to separate different ligation products for different loci from one another. Thus, in certain embodiments, both ligation probe sets for a single locus, such as AZ and BW for locus L1, will have the same addressable portion. Also, in certain such embodiments, the two different first probes, e.g., Probe A and Probe B, comprise different labels. Thus, in certain embodiments, both methylation states for a given locus may be detected at the same position on a solid support. Therefore, in certain embodiments, the labels for target nucleotides of locus L1 will be detected at position 1, the labels for target nucleotides of locus L2 will be detected at position 2, and the labels for target nucleotides of locus L3 will be detected at position 3.

For example, in certain embodiments, a red label may be associated with a methylated nucleotide and a blue label may be associated with an unmethylated nucleotide. In certain such embodiments, a sample may result in a red label at position 1, a blue label at position 2, and both red and blue labels at position 3. In such a case, one may conclude that such a sample includes methylated nucleotides at locus L1, unmethylated nucleotides at locus L2, and both methylated and unmethylated nucleotides at locus L3.

The skilled artisan will understand that in certain embodiments, the probes can be designed with the test nucleotide at any location in either the first ligation probe or the second ligation probe. Additionally, in certain embodiments, ligation probes comprising multiple test nucleotides are within the scope of the present teachings.

Exemplary Methods for Detecting Nucleotide Differences

Certain embodiments are directed to methods, reagents, and kits for detecting the identity of a nucleotide. In certain such embodiments, it is possible that different target nucleic acid sequences in a sample have different nucleotides at a given test position. In certain embodiments, one employs a ligation reaction comprising a ligation probe set and a blocking probe that results in a given ligation product only if the first nucleotide at a test position is present in a sample. In certain embodiments, ligation products may form even if the appropriate target nucleic acid sequence comprising the first nucleotide at a test position is not in the sample, but such ligation occurs to a measurably lesser extent than when the appropriate target nucleic acid sequence is in the sample.

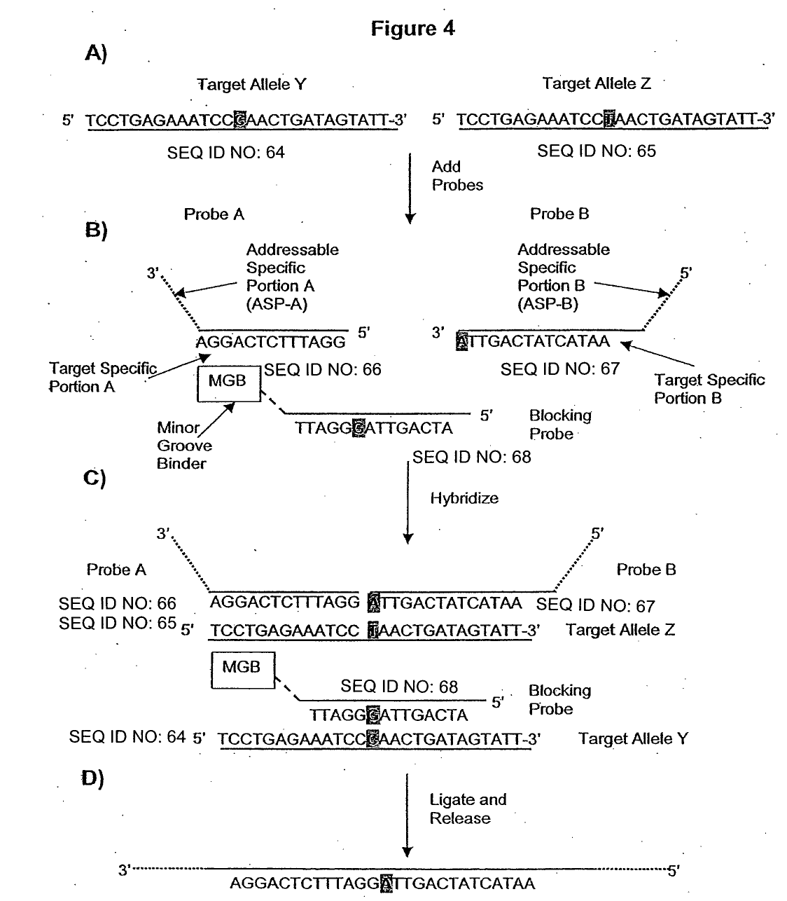

In certain embodiments, a ligation probe set and blocking probe are provided for detecting a first nucleotide at a test position in at least one first target nucleic acid sequence in a sample, wherein the sample comprises at least one second target nucleic acid sequence comprising a second different nucleotide at the test position. See, e.g., FIG. 4. In certain embodiments, the ligation probe set comprises: (a) a first probe, comprising a first target-specific portion; and (b) a second probe, comprising a second target-specific portion. See, e.g., Probes A and B in FIG. 4(B). In certain embodiments the first and second probes are subjected to at least one cycle of ligation, under conditions effective to ligate together first and second probes that are hybridized adjacent to one another on the first target nucleic acid sequence if the first nucleotide is present at the test position to form a ligation product. See, e.g., FIGS. 4(C) and 4(D). In certain embodiments, a blocking probe hybridizes to a portion of the second target nucleic acid sequence comprising the second different nucleotide at the test position. See, e.g., FIG. 4(C). In certain embodiments, the blocking probe comprises a minor groove binder (shown as “MGB” in FIG. 4) or other moiety which increases the Tₘ and enhances mismatch discrimination. In certain such embodiments, hybridization of the blocking probe to the portion of the second target nucleic acid sequence blocks hybridization of the first probe, the second probe, or both the first probe and the second probe to the portion of the second target nucleic acid sequence. In certain embodiments, one detects the presence of the first nucleotide in the first target nucleic acid sequence by detecting a ligation product. See, e.g., FIG. 4(D). In certain embodiments, the test position is a Single Nucleotide Polymorphism (SNP) site.

In certain embodiments, multiple ligation probe-sets and blocking probes may be used to detect two or more different nucleotides at a single test position. For example, in certain embodiments, where one seeks to detect two different nucleotides at a single test position, one may prepare two separate reaction compositions comprising a portion of the sample and the ligation probe sets and blocking probes as shown below in Table 3:

TABLE 3

| | Nucleotide | Ligation Probe Set/Blocking Probe |
|---|---|---|
| Reaction | First | Probe A (red)--Probe Z (AP 1) |
| Composition 1 | Nucleotide | |
| | Second | Blocking Probe BW |
| | Nucleotide | |
| Reaction | First | Blocking Probe AZ |
| Composition 2 | Nucleotide | |
| | Second | Probe B (blue)--Probe W (AP 1) |
| | Nucleotide | |

AP = Addressable Portion

In certain embodiments, as shown in Table 3, one prepares a first reaction composition comprising a ligation probe set specific for a first nucleotide at a test position in at least one first target nucleic acid sequence, e.g., Probe A and Probe Z of Table 3. In certain such embodiments, the first reaction composition further comprises a blocking probe specific for a second different nucleotide at the test position in at least one second target nucleic acid sequence, e.g., Blocking Probe BW. In certain embodiments, one prepares a second reaction composition comprising a ligation probe set specific for the second different nucleotide at the test position in at least one second target nucleic acid sequence, e.g., Probe B and Probe W. In certain such embodiments, the second reaction composition further comprises a blocking probe specific for the first nucleotide at the test position in at least one first target nucleic acid sequence, e.g., Blocking Probe AZ. In certain embodiments, the ligation product formed by Probe A and Probe Z is distinguishable from the ligation product formed by Probe B and Probe W such that the ligation products from the first reaction composition and the second reaction composition can be combined and analyzed simultaneously.

In certain embodiments, three or more different nucleotides may be found at the same test position in three or more different target nucleic acid sequences. In certain such embodiments, a reaction composition comprises two or more different blocking probes. In certain embodiments, a reaction composition comprises a blocking probe for each different nucleotide at a test position other than the nucleotide at the test position which is being detected by a ligation probe set.

In certain embodiments, multiple ligation probe sets and blocking probes may be used to detect multiple different nucleotides at test positions of multiple different loci. In certain such embodiments, one may employ multiple ligation probe sets that each include: different first probes that comprise different addressable portions and/or different second probes that comprise different addressable portions. In certain embodiments, the different addressable portions are used to separate ligation products for the different loci being analyzed.

In certain embodiments, the ligation reaction composition may comprise different ligation probe sets and different blocking probes for detecting multiple different nucleotides at test positions of multiple loci. In certain embodiments, one may, for example, without limitation, detect three different nucleotides at test positions of three different loci (e.g., L1, L2, and L3) using three ligation probe sets and three blocking probes. See, e.g., Table 4 below.

TABLE 4

| Locus | Meth. State | Ligation Probe Set/Blocking Probe |
|---|---|---|
| L1 | First Nucleotide | Probe A (red)-Probe Z (AP 1) |
| | Second Nucleotide | Blocking Probe BW |
| L2 | First Nucleotide | Probe C (red)-Probe Y (AP 2) |
| | Second Nucleotide | Blocking Probe DV |
| L3 | First Nucleotide | Probe E (red)-Probe X (AP 3) |
| | Second Nucleotide | Blocking Probe FU |

AP = Addressable Portion

In certain embodiments, one uses the three ligation probe sets described in Table 4 to detect nucleotides at the test positions of three loci L1, L2, and L3. In certain such embodiments, one detects a target nucleotide at the test position by detecting the presence of the appropriate ligation product. For example, in certain embodiments, one detects the first nucleotide at the test position of locus L1 by detecting the AZ ligation product. In certain such embodiments, one infers that the second nucleotide is present at the test position of L1 if one does not detect AZ ligation product. In certain such embodiments, one detects the first nucleotide at the test position of locus L2 by detecting CY ligation product. In certain such embodiments, one infers that the second nucleotide is present at the test position of L2 if one does not detect CY ligation product. In certain such embodiments, one detects the first nucleotide at the test position of locus L3 by detecting EX ligation product. In certain such embodiments, one infers that the second nucleotide is present at the test position of L2 if one does not detect EX ligation product.

In certain embodiments, one may test a sample using two separate reaction compositions with two different groups of ligation probe sets and two different groups of blocking probes. For example, in certain embodiments, one may use a first ligation reaction composition comprising a portion of the sample and the ligation probe sets and blocking probes in Table 4 above. In certain such embodiments, one may also use a separate second ligation reaction composition comprising another portion of the sample and the ligation probe sets and blocking probes in Table 5 below.

TABLE 5

| Locus | Meth. State | Ligation Probe Set/Blocking Probe |
|---|---|---|
| L1 | First Nucleotide | Blocking Probe AZ |
| | Second Nucleotide | Probe B (blue)--Probe W (AP 1) |
| L2 | First Nucleotide | Blocking Probe CY |
| | Second Nucleotide | Probe D (blue)--Probe V (AP 2) |
| L3 | First Nucleotide | Blocking Probe EX |
| | Second Nucleotide | Probe F (blue)--Probe U (AP 3) |

AP = Addressable Portion

Thus, in certain embodiments, two different groups of ligation probe sets and two different groups of blocking probes are used in two different reaction compositions to detect the two different nucleotides at the test positions of each locus. For example, and not limitation, in certain embodiments, the two first ligation probes of the two different ligation probe sets for each locus, for example, probes A and B for locus L1, comprise the same target-specific portion, but differ at one or more test nucleotides. In certain embodiments, the two second ligation probes of the two different ligation probe sets for each locus, for example, probes Z and W for locus L1, comprise the same target-specific portion, but differ at one or more test nucleotides.

Thus, in certain embodiments, such as the embodiments depicted in Tables 4 and 5, four probes A, B, Z, and W are used to form the two possible L1 ligation products. In certain such embodiments, an AZ ligation product indicates that the first nucleotide is at the test position of locus L1 and a BW ligation product indicates that the second nucleotide is at the test position of locus L1. Likewise, probes C, D, V, and Y, are used to form the two possible L2 ligation products. Likewise, probes E, F, U, and X, are used to form the two possible L3 ligation products.

After ligation of adjacently hybridized first and second ligation probes, in certain embodiments, one can detect the presence or absence of a ligation product for each different nucleotide at the test position of each loci by using unique combinations of labels and addressable portions. For example, in certain embodiments, one may detect the different nucleotides at the test position of locus L1 by the label or labels detected at position 1 in view of addressable portion 1, detect the different nucleotides at the test position of locus L2 by the label or labels detected at position 2 in view of addressable portion 2, and detect the different nucleotides at the test position of locus L3 by the label or labels detected at position 3 in view of addressable portion 3.

For example and not limitation, in certain embodiments, the probes Z and W comprise the same addressable portion. In certain embodiments, the addressable portion of the second ligation probes is the same for all second ligation probes directed to the same locus, but is different for each different locus. Thus, in certain embodiments, each different addressable portion may be used to separate different ligation products for different loci from one another. Thus, in certain embodiments, both ligation probe sets for a single locus, such as AZ and BW for locus L1, will have the same addressable portion. Also, in certain such embodiments, the two different first probes, e.g., Probe A and Probe B, comprise different labels. Thus, in certain embodiments, different nucleotides at a test position for a given locus may be detected at the same position on a solid support. Therefore, in certain embodiments, the labels for nucleotides of locus L1 will be detected at position 1, the labels for nucleotides of locus L2 will be detected at position 2, and the labels for nucleotides of locus L3 will be detected at position 3.

For example, in certain embodiments, a red label may be associated with a first nucleotide and a blue label may be associated with a second nucleotide. In certain such embodiments, a sample may result in a red label at position 1, a blue label at position 2, and both red and blue labels at position 3. In such a case, one may conclude that such a sample includes the first nucleotide at the test position of locus L1, the second nucleotide at the test position of locus L2, and both the first nucleotide and the second nucleotide at the test position of locus L3.

The skilled artisan will understand that in certain embodiments, the probes can be designed with the test nucleotide at any location in either the first ligation probe or the second ligation probe. Additionally, in certain embodiments, ligation probes comprising multiple test nucleotides are within the scope of the present teachings.

In certain embodiments, one may perform multiplex assays which detect three or more target nucleotides at one or more test positions by setting up reaction compositions for each target nucleotides. In certain such embodiments, one designs a ligation probe set for detecting each target nucleotide and two or more blocking probes for use with each ligation probe set.

Oligonucleotide Ligation and Amplification

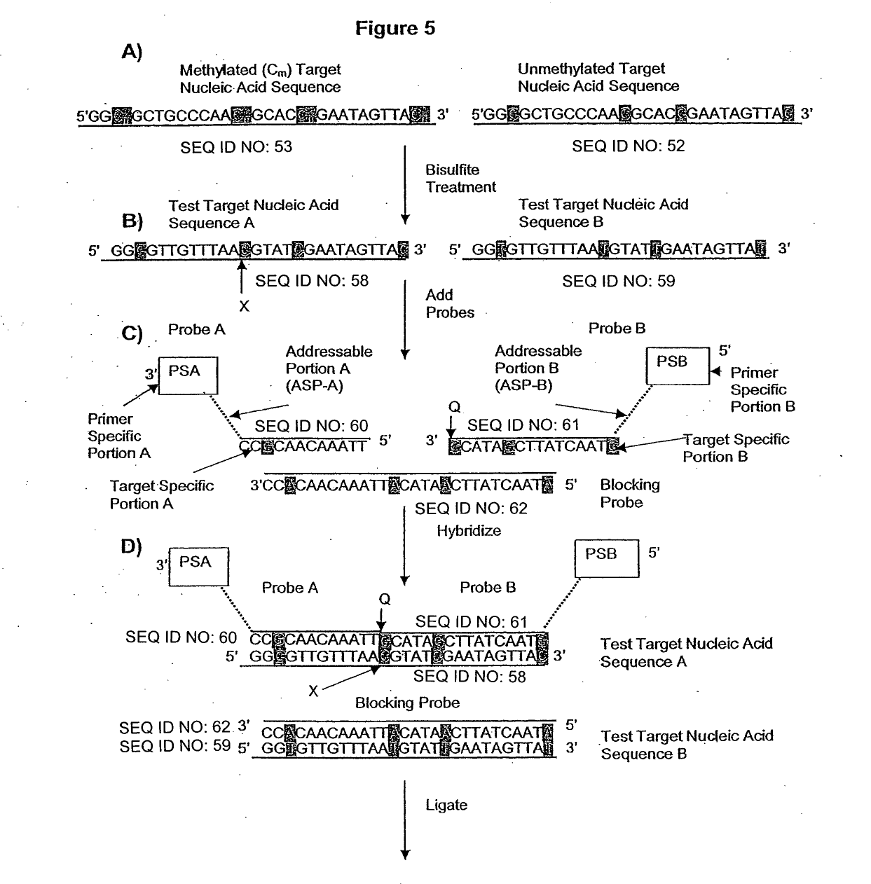

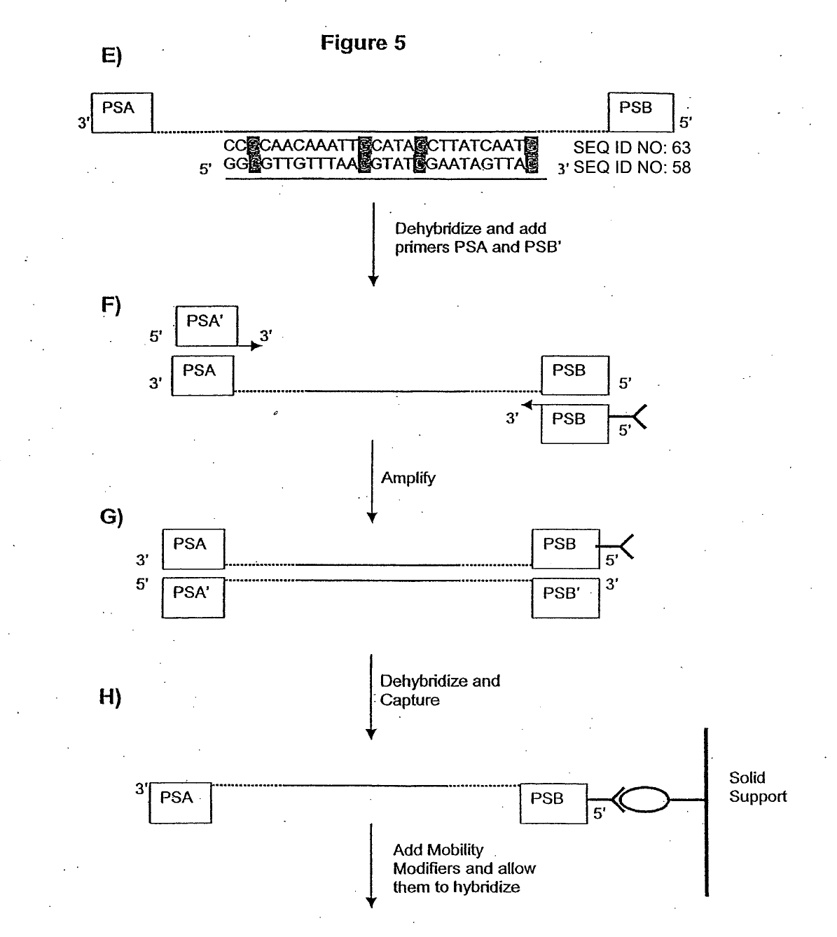

In certain embodiments, one employs a ligation reaction followed by amplification to obtain polynucleotides to detect target nucleic acids. A nonlimiting example, shown in FIG. 5, features a method comprising exposing Target Nucleic Acid Sequence to a modifying agent to obtain Test Target Nucleic Acid Sequence. See, e.g., FIGS. 5(A) and (B). However, the amplification and detection steps of FIG. 5 can be adapted to other embodiments comprising ligation probe sets and blocking probes.

In the embodiment shown in FIG. 5, Methylated (Cₘ) Target Nucleic Acid Sequence is exposed to bisulfite treatment to obtain Test Target Nucleic Acid Sequence A, and Unmethylated Target Nucleic Acid Sequence is exposed to bisulfite treatment to obtain Test Target Nucleic Acid Sequence B. In the embodiments shown in FIG. 5, the first and second probes in each ligation probe set are designed to be complementary to the sequences immediately flanking a target nucleotide (X) of the test target nucleic acid sequence (see, e.g., Probes A and B in FIGS. 5(C) and (D)). In the embodiment shown in FIG. 5, a first probe, Probe A, of a ligation probe set comprises a Primer Specific Portion A (PSA) and an Addressable Portion A (ASP-A). In the embodiment shown in FIG. 5, the second probe, Probe B, of the ligation probe set comprises: (1) a test nucleotide (Q) that base pairs with the target nucleotide X; (2) a different primer specific portion, Primer Specific Portion B (PSB); and (3) a different addressable portion, Addressable Portion (ASP-B). In the embodiment shown in FIG. 5, the particular combination of ASP-A and ASP-B corresponds to the locus being analyzed.

In the embodiment shown in FIG. 5, Probe A and Probe B hybridize to Test Target Nucleic Acid Sequence A obtained after bisulfite treatment of Methylated (Cₘ) Target Nucleic Acid Sequence (see, e.g. FIGS. 5(A), (B), (C), and (D) showing Test Target Nucleic Acid Sequence A, Probe A, and Probe B). Although, Probe A and Probe B can also align to Test Target Nucleic Acid Sequence B, both Probe A and Probe B cannot do so without creating mismatches corresponding to the positions of the unmethylated target cytosines in the Unmethylated Target Nucleic Acid Sequence (See, e.g., FIGS. 5(B) and (C) showing Test Target Nucleic Acid Sequence B, Probe A, and Probe B). In the embodiment shown in FIG. 5, when probe A and probe B hybridize to Test Target Nucleic Acid Sequence A, they align adjacent to each other such that they may be ligated together under the appropriate conditions (see, e.g., FIG. 5(D)).

The embodiment shown in FIG. 5, also shows a blocking probe which hybridizes to Test Target Nucleic Acid Sequence B. Although, the blocking probe can also align to Test Target Nucleic Acid Sequence A, it cannot do so without creating four mismatches corresponding to the positions of the methylated target cytosines in the Methylated (Cₘ) Target Nucleic Acid Sequence (See, e.g., FIGS. 5(B) and (C) showing Test Target Nucleic Acid Sequence A and the Blocking Probe). When the blocking probe in the embodiment shown in FIG. 5 hybridizes to Test Target Nucleic Acid Sequence B, it prevents Probe A and Probe B from binding to that test target nucleic acid. Thus, in the embodiment shown in FIG. 5, the Blocking Probe blocks hybridization of Probe A and Probe B to Test Target Nucleic Acid Sequence B.

In certain embodiments, a first amplification reaction is formed comprising: the ligation product; at least one primer set; the appropriate salts, buffers, and nucleotide triphosphates; and a polymerase. See, e.g., the primer set comprising PSA′ and PSB in FIG. 5(F). In the first amplification cycle, the primer PSA′, comprising a sequence complementary to the 3′ primer-specific portion of the ligation product, hybridizes with the ligation product and is extended in a template-dependent fashion to create a double-stranded molecule comprising the ligation product and its complement. See, e.g., FIGS. 5(F)-(G). Subsequent amplification cycles may exponentially amplify this double-stranded molecule. In certain embodiments, the two different primers include different labels. Thus, amplification products resulting from incorporation of these primers will include a combination of labels specific for the particular target nucleotide that is included in the original target sequence.

In certain embodiments, after amplification of ligation products, amplification products may be captured on a solid support. See, e.g., FIG. 5(H). In certain embodiments, a mobility modifier comprising a label (⋄); a polymer tail; and a complementary addressable portion (ASP-A′), hybridizes to the amplification product bound to the solid support. See, e.g., FIG. 5(I). In certain embodiments, mobility modifiers not hybridized to the amplification product are washed away. See, e.g., FIG. 5(J). In certain embodiments, the mobility modifiers hybridized to the amplification product are released from the amplification product and detected. See, e.g., FIG. 5(K).

In certain embodiments, the addressable portions may be hybridized to appropriate mobility modifiers and then the presence of particular ligation products may be detected using a mobility dependent analysis technique. In certain embodiments, for example and not limitation, where one is performing a multiplex assay with multiple ligation probe sets, the different ligation probe sets may all have the same first primer specific portions and the same second primer specific portions. In certain such embodiments, the different ligation probe sets may have one or more addressable portions which are distinctive for that ligation probe set. In certain such embodiments, the addressable portion is located between the primer specific portion and the target specific portion of one of the probes of the ligation probe set.

Certain Exemplary Kits

In certain embodiments, kits designed to expedite performing certain methods are also provided. In certain embodiments, kits serve to expedite the performance of the methods of interest by assembling two or more components used in carrying out the methods. In certain embodiments, kits may contain components in pre-measured unit amounts to minimize the need for measurements by end-users. In certain embodiments, kits may include instructions for performing one or more methods of the present teachings. In certain embodiments, the kit components are optimized to operate in conjunction with one another.

In certain embodiments, kits for determining the methylation state of a target nucleotide in at least one target nucleic acid sequence in a sample comprise: at least one blocking probe; a ligation probe set for each target nucleotide, the ligation probe set comprising: (a) at least one first probe, comprising a first target-specific portion, and (b) at least one second probe, comprising a second target-specific portion, wherein the probes in each set are suitable for ligation together when hybridized adjacent to one another on a complementary target nucleic acid sequence; and a ligase. In certain embodiments, the ligase is a selective ligase that ligates together adjacently hybridized probes to a measurably lesser extent if the target nucleotide is unmethylated. In certain embodiments, the ligase is a selective ligase that ligates together adjacently hybridized probes to a measurably lesser extent if the target nucleotide is methylated.

In certain embodiments, the at least one first probe further comprises a 5′ primer-specific portion, wherein the 5′ primer-specific portion comprises a first sequence, and the at least one second probe further comprises a 3′ primer-specific portion, wherein the 3′ primer-specific portion comprises a second sequence. In certain embodiments, the kit further comprises: at least one primer set, the primer set comprising (i) at least one first primer comprising the first sequence of the 5′ primer-specific portion of the first probe, and (ii) at least one second primer comprising a sequence complementary to the second sequence of the 3′ primer-specific portion of the second probe.

In certain embodiments, kits for determining the methylation state of a target nucleotide in at least one target nucleic acid sequence in a sample comprise a modifying agent that modifies methylated target nucleotide, but does not modify unmethylated target nucleotide. In certain embodiments, kits for determining the methylation state of a target nucleotide in at least one target nucleic acid sequence in a sample comprise a modifying agent that modifies unmethylated target nucleotide, but does not modify methylated target nucleotide. In various embodiments, kits for determining the methylation state of a target nucleotide in at least one target nucleic acid sequence in a sample comprise: at least one blocking probe; and a ligation probe set for each target nucleotide, the ligation probe set comprising: (a) at least one first probe, comprising a first target-specific portion, and (b) at least one second probe, comprising a second target-specific portion, wherein the probes in each set are suitable for ligation together when hybridized adjacent to one another on a complementary target nucleic acid sequence.

In certain embodiments, the at least one first probe further comprises a 5′ primer-specific portion, wherein the 5′ primer-specific-portion comprises a first sequence, and the at least one second probe further comprises a 3′ primer-specific portion, wherein the 3′ primer-specific portion comprises a second sequence. In certain embodiments, the kit further comprises: at least one primer set, the primer set comprising (i) at least one first primer comprising the first sequence of the 5′ primer-specific portion of the first probe, and (ii) at least one second primer comprising a second sequence complementary to the sequence of the 3′ primer-specific portion of the second probe.

In certain embodiments, kits for detecting a first nucleotide at a test position in at least one first target nucleic acid sequence in a sample comprise: at least one blocking probe comprising at least one modification, wherein the modification either: (a) increases the affinity of the blocking probe for a nucleic acid sequence that is complementary to the blocking probe sequence without any mismatches, (b) decreases the affinity of the blocking probe for a nucleic acid sequence that differs by at least one nucleotide from a sequence that is complementary to the blocking probe sequence without any mismatches, or (c) both increases the affinity of the blocking probe for a nucleic acid sequence that is exactly complementary to the blocking probe sequence without any mismatches and decreases the affinity of the blocking probe for a nucleic acid sequence that differs by at least one nucleotide from a sequence that is complementary to the blocking probe without any mismatches; and a ligation probe set for the first target nucleic acid sequence, the ligation probe set comprising: (a) at least one first probe, comprising a first target-specific portion, and (b) at least one second probe, comprising a second target-specific portion, wherein the first probe and second probe in each ligation probe set are suitable for ligation together when hybridized adjacent to one another on the first target nucleic acid sequence.

In certain embodiments, the at least one first probe further comprises a 5′ primer-specific portion, wherein the 5′ primer-specific-portion comprises a first sequence, and the at least one second probe further comprises a 3′ primer-specific portion, wherein the 3′ primer-specific portion comprises a second sequence. In certain embodiments, the kit further comprises: at least one primer set, the primer set comprising (i) at least one first primer comprising the first sequence of the 5′ primer-specific portion of the first probe, and (ii) at least one second primer comprising a sequence complementary to the second sequence of the 3′ primer-specific portion of the second probe.

In certain embodiments, kits comprise one or more additional components, including, without limitation, at least one of: at least one polymerase, at least one ligation agent, oligonucleotide triphosphates, nucleotide analogs, reaction buffers, salts, ions, and stabilizers. In certain embodiments, kits comprise one or more reagents for purifying the ligation products, including, without limitation, at least one of dialysis membranes, chromatographic compounds, supports, and oligonucleotides.

Aspects of the present teachings may be further understood in light of the following examples, which should not be construed as limiting the scope the present teachings in any way.

### EXAMPLE 1

Preparation of Unmethylated Genomic DNA

300 ng of unmethylated genomic DNA (1 μl of Coriell genomic DNA) was added to 44 μl of water and 5 μl of M-dilution buffer (Zymo Research, Orange Calif.) to form a bisulfite pre-reaction composition. The bisulfite pre-reaction composition was incubated at 37° C. for 15 minutes.

A bisulfite stock solution (approximately 3M bisulfite) was prepared by adding 210 μl of the M-dilution buffer (Zymo Research) and 750 μl of water to a vial provided by Zymo Research (Zymo Research), which contained Zymo CT conversion reagent. After adding the M-dilution buffer and the water, the bisulfite stock solution was vortexed periodically over a 10 minute period to dissolve the Zymo CT conversion reagent. After dissolving the Zymo CT conversion reagent, 100 μl of the bisulfite stock solution was removed and added to the bisulfite pre-reaction composition to form a bisulfite reaction composition. The bisulfite reaction composition was incubated for 15 hours at 50° C.

Following the incubation, 300 μl of water was added to the bisulfite reaction composition, and the resulting solution was transferred to the upper chamber of a Microcon 100 filtration device (Millipore, Billerica Mass.), which was centrifuged at 2800 rpm for 18 minutes. The filtrate was removed and 350 μl of water was added to the upper chamber of the filtration device. The filtration device was centrifuged at 2800 rpm for 15 minutes. The filtrate was removed and another 350 μl of water was added to the upper chamber of the filtration device. The filtration device was centrifuged at 2800 rpm for 15 minutes. The filtrate was removed and 350 μl of 0.1 M NaOH was added to the upper chamber of the filtration device. After adding the NaOH, the filtration device was allowed to sit for 5 minutes, and was then centrifuged at 2800 rpm for 15 minutes. The filtrate was removed and 350 μl of water was added to the upper chamber of the filtration device. The filtration device was centrifuged at 2800 rpm for 15 minutes. The filtrate was removed and 50 μl of TE buffer (10 mM Tris Cl; 1 mM EDTA (pH 8.0) (Teknova, T0223)) was added to the upper chamber of the filtration device with gentle mixing. The filtration device was allowed to sit for 5 minutes after which the device was inverted and the TE buffer containing the bisulfite treated DNA was collected.

Preparation of Methylated Genomic DNA

300 ng of methylated genomic DNA (3.3 μl of CpGenome universal methylated DNA (Serologicals P/N S7821, Norcross, Ga.)) was added to 44 μl of water and 5 μl of M-dilution buffer (Zymo Research) to form a bisulfite pre-reaction composition. The bisulfite pre-reaction composition was incubated at 37° C. for 15 minutes.

A bisulfite stock solution (approximately 3M bisulfite) was prepared by adding 210 μl of the M-dilution buffer (Zymo Research) and 750 μl of water to a vial provided by Zymo Research (Zymo Research), which contained Zymo CT conversion reagent. After adding the M-dilution buffer and the water, the bisulfite stock solution was vortexed periodically over a 10 minute period to dissolve the Zymo CT conversion reagent. After dissolving the Zymo CT conversion reagent, 100 μl of the bisulfite stock solution was removed and added to the bisulfite pre-reaction composition to form a bisulfite reaction composition. The bisulfite reaction composition was incubated for 15 hours at 50° C.

Following the incubation, 300 μl of water was added to the bisulfite reaction composition, and the resulting solution was transferred to the upper chamber of a Microcon 100 filtration device (Millipore), which was centrifuged at 2800 rpm for 18 minutes. The filtrate was removed and 350 μl of water was added to the upper chamber of the filtration device. The filtration device was centrifuged at 2800 rpm for 15 minutes. The filtrate was removed and another 350 μl of water was added to the upper chamber of the filtration device. The filtration device was centrifuged at 2800 rpm for 15 minutes. The filtrate was removed and 350 μl of 0.1 M NaOH was added to the upper chamber of the filtration device. After adding the NaOH, the filtration device was allowed to sit for 5 minutes, and was then centrifuged at 2800 rpm for 15 minutes. The filtrate was removed and 350 μl of water was added to the upper chamber of the filtration device. The filtration device was centrifuged at 2800 rpm for 15 minutes. The filtrate was removed and 50 μl of TE buffer (10 mM Tris Cl; 1 mM EDTA (pH 8.0) (Teknova, T0223)) was added to the upper chamber of the filtration device with gentle mixing. The filtration device was allowed to sit for 5 minutes after which the device was inverted and the TE buffer containing the bisulfite treated DNA was collected.

PCR Amplification of Bisulfite Treated DNA

Bisulfite treated unmethylated genomic DNA was amplified using the polymerase chain reaction with primers P15 Forward Primer (TAGGTTTTTTAGGAAGGAGAGAGTG (SEQ ID NO.: X)) and P15 Reverse Primer (CTAAAACCCCAACTACCTAAAT (SEQ ID NO.: X)) to generate target DNA from the P15 gene (“P15 unmethylated target DNA”).

PCR Reaction Composition

| | |
|---|---|
| 2X Taq Gold PCR Master Mix (Applied Biosystems, | 10 μl |
| P/N 4326717) | |
| Aliquot of bisulfite treated unmethylated genomic | 1 μl |
| DNA (5 ng/μl) | |
| P15 Forward Primer (5 μM) | 1 μl |
| P15 Reverse Primer (5 μM) | 1 μl |
| Water | 7 μl |
| total volume | 20 μl |

PCR reaction compositions were heated to 95° C. for 5 minutes, and were then incubated under thermal cycling conditions. The thermal cycling conditions were 95° C. for 30 seconds, followed by 60° C. for 45 seconds, followed by 72° C. for 1 minute. Those cycling conditions were repeated 40 times. After the PCR reaction compositions were incubated under thermal cycling conditions, those reaction compositions were maintained at 4° C.

Following PCR amplification, an Exo/Sap Solution was made as follows.

Exo/Sap Solution

| | |
|---|---|
| Shrimp Alkaline Phosphatase (SAP) (1 U/μl) | 2 μl |
| (USB, P/N 4326717) | |
| Exonuclease I (10 U/μl) | 0.2 μl |
| Water | 1.8 μl |
| total volume | 4 μl |

The 4 μl Exo/Sap Solution was then added to the 20 pt PCR Reaction Composition to form an Exo/SAP Digestion Composition. The Exo/SAP Digestion Composition was heated to 37° C. for 1 hour, and then heated to 72° C. for 15 minutes.

Bisulfite treated methylated genomic DNA was amplified using the polymerase chain reaction with the P15 Forward Primer and the P15 Reverse Primer to generate target DNA from the P15 gene (“P15 methylated target DNA”).

PCR Reaction Composition

| | |
|---|---|
| 2X Taq Gold PCR Master Mix (Applied Biosystems, | 10 μl |
| P/N 4326717) | |
| Aliquot of bisulfite treated methylated genomic | 1 μl |
| DNA (5 ng/μl) | |
| P15 Forward Primer (5 μM) | 1 μl |
| P15 Reverse Primer (5 μM) | 1 μl |
| Water | 7 μl |
| total volume | 20 μl |

PCR reaction compositions were heated to 95° C. for 5 minutes, and were then incubated under thermal cycling conditions. The thermal cycling conditions were 95° C. for 30 seconds, followed by 60° C. for 45 seconds, followed by 72° C. for 1 minute. Those cycling conditions were repeated 40 times. After the PCR reaction compositions were incubated under thermal cycling conditions, those reaction compositions were maintained at 4° C.

Following PCR amplification, an Exo/Sap Solution was made as follows.

Exo/Sap Solution

| | |
|---|---|
| Shrimp Alkaline Phosphatase (SAP) (1 U/μl) | 2 μl |
| (USB, P/N 4326717) | |
| Exonuclease I (10 U/μl) | 0.2 μl |
| Water | 1.8 μl |
| total volume | 4 μl |

The 4 μl Exo/Sap Solution was then added to the 20 μl PCR Reaction Composition to form an Exo/SAP Digestion Composition. The Exo/SAP Digestion Composition was heated to 37° C. for 1 hour, and then heated to 72° C. for 15 minutes.

Following PCR amplification and the Sap/Exo treatment, an equal volume aliquot from the amplification of the P15 unmethylated (UnMe) target DNA and the P15 methylated (Me) target DNA were mixed to form the “P15 template.”

Oligo Ligation Assay (OLA) Analysis of the P15 Template

The following probes were designed for the following OLA reactions:

| | |
|---|---|
| P15 Me P B-OLA: | |
| CGACGCTAACCAAACCC | (SEQ ID NO.: X) |
| P15 Me FAM B-OLA: | |
| (FAM)-CTAATCCCCGCGCCG | (SEQ ID NO.: X) |
| P15 Blocking Probe: | |
| CCCACACCACAACACTAACC | (SEQ ID NO.: X) |
| P15 UnMe P: | |
| CAACACTAACCAAACCC | (SEQ ID NO.: X) |
| P15 UnMe ASO: | |
| (FAM)-CTAATCCCCACACCA | (SEQ ID NO.: X) |

The probes P15 Me P B-OLA and P15 Me FAM B-OLA were a ligation probe set designed to hybridize to the P15 methylated target DNA adjacent to each other.

The probes P15 UnMe P and P15 UnMe ASO were a ligation probe set designed to hybridize to the P15 unmethylated target DNA adjacent to each other as a ligation probe set.

The probe P15 Blocking Probe was designed to hybridize to the P15 unmethylated target DNA in such a manner that it would compete with the ligation probe sets for binding to the P15 unmethylated target DNA.

The probes P15 Me P B-OLA, P15 Me FAM B-OLA, P15 UnMe P, P15 UnMe ASO, and P15 Blocking Probe were each designed to have a Tm of approximately 60° C.

The following reaction compositions comprising the P15 Me P B-OLA and P15 Me FAM B-OLA probes were prepared:

Reaction Composition (5 μM Blocking Probe)

| | |
|---|---|
| Taq Ligase buffer 10X | 1 μl |
| Taq Ligase (4 U/μl) | 1 μl |
| P15 Template (50:50 UnMe:Me) | 1 μl |
| P15 Me FAM B-OLA (0.5 μM) | 1 μl |
| P15 Me P B-OLA (0.5 μM) | 1 μl |
| P15 Blocking Probe (5 μM) | 1 μl |
| Water | 4 μl |
| total volume | 10 μl |

Reaction Composition (0.5 μM Blocking Probe)

| | |
|---|---|
| Taq Ligase buffer 10X | 1 μl |
| Taq Ligase (4 U/μl) | 1 μl |
| P15 Template (50:50 UnMe:Me) | 1 μl |
| P15 Me FAM B-OLA (0.5 μM) | 1 μl |
| P15 Me P B-OLA (0.5 μM) | 1 μl |
| P15 Blocking Probe (0.5 μM) | 1 μl |
| Water | 4 μl |
| total volume | 10 μl |

Reaction Composition (0 μM Blocking Probe)

| | |
|---|---|
| Taq Ligase buffer 10X | 1 μl |
| Taq Ligase (4 U/ul) | 1 μl |
| P15 Template (50:50 UnMe:Me) | 1 μl |
| P15 Me FAM B-OLA (0.5 μM) | 1 μl |
| P15 Me P B-OLA (0.5 μM) | 1 μl |
| P15 Blocking Probe (0 μM) | 1 μl |
| Water | 4 μl |
| total volume | 10 μl |

The following reaction compositions comprising the P15 UnMe P and P15 UnMe ASO probes were prepared:

Reaction Composition (5 μM Blocking Probe)

| | |
|---|---|
| Taq Ligase buffer 10X | 1 μl |
| Taq Ligase (4 U/μl) | 1 μl |
| P15 Template (50:50 UnMe:Me) | 1 μl |
| P15 UnMe ASO (0.5 μM) | 1 μl |
| P15 UnMe P (0.5 μM) | 1 μl |
| P15 Blocking Probe (5 μM) | 1 μl |
| Water | 4 μl |
| total volume | 10 μl |

Reaction Composition (0.5 μM Blocking Probe)

| | |
|---|---|
| Taq Ligase buffer 10X | 1 μl |
| Taq Ligase (4 U/μl) | 1 μl |
| P15 Template (50:50 UnMe:Me) | 1 μl |
| P15 UnMe ASO (0.5 μM) | 1 μl |
| P15 UnMe P (0.5 μM) | 1 μl |
| P15 Blocking Probe (0.5 μM) | 1 μl |
| Water | 4 μl |
| total volume | 10 μl |

Reaction Composition (0 μM Blocking Probe)

| | |
|---|---|
| Taq Ligase buffer 10X | 1 μl |
| Taq Ligase (4 U/μl) | 1 μl |
| P15 Template (50:50 UnMe:Me) | 1 μl |
| P15 UnMe ASO (0.5 μM) | 1 μl |
| P15 UnMe P (0.5 μM) | 1 μl |
| P15 Blocking Probe (0 μM) | 1 μl |
| Water | 4 μl |
| total volume | 10 μl |

Reaction compositions were incubated under thermal cycling conditions. The thermal cycling conditions were 95° C. for 5 seconds followed by 60° C. for 1 minute. Those cycling conditions were repeated 60 times.

Following incubation under thermal cycling conditions, a 1.2 A1 aliquot of the solution was removed and mixed with 12 μl of Hi-Di Formamide (Applied Biosystems, Foster City Calif.). The resulting Formamide solution was heated to 95° C. for five minutes, and the samples were then analyzed by capillary electrophoresis on an ABI PRISM 310 Genetic Analyzer (Applied Biosystems).

Results for the reaction compositions comprising the P15 Me P B-OLA and P15 Me FAM B-OLA primers are shown in FIG. 5.

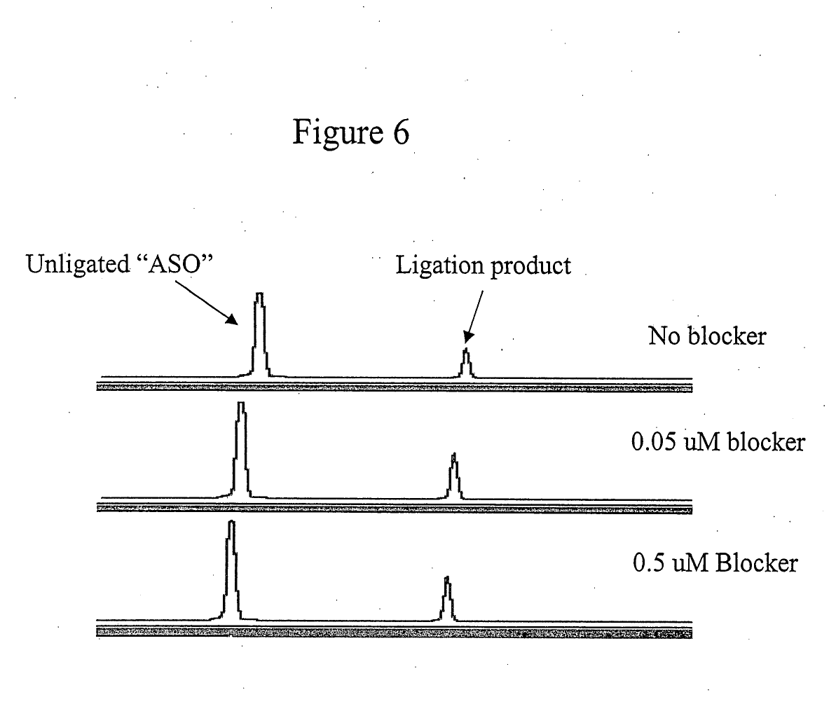

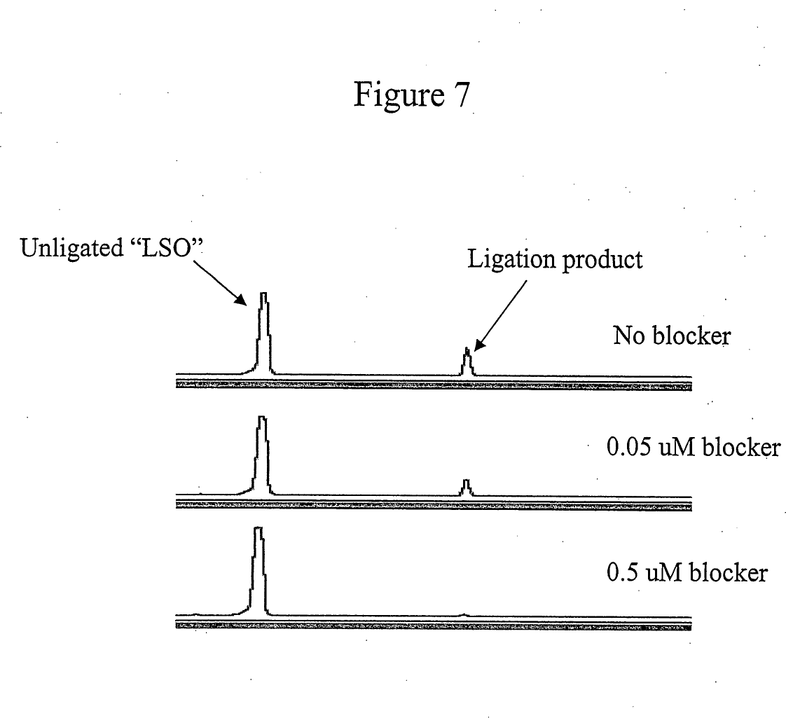

Results for the reaction compositions comprising the P15 UnMe P and P15 UnMe ASO primers are shown in FIG. 6.

### EXAMPLE 2

Preparation of Unmethylated Genomic DNA

300 ng of unmethylated genomic DNA (1 μl of Coriell genomic DNA) was added to 44 μl of water and 5 μl of M-dilution buffer (Zymo Research) to form a bisulfite pre-reaction composition. The bisulfite pre-reaction composition was incubated at 37° C. for 15 minutes.

A bisulfite stock solution (approximately 3M bisulfite) was prepared by adding 210 μl of the M-dilution buffer (Zymo Research) and 750 μl of water to a vial provided by Zymo Research (Zymo Research), which contained Zymo CT conversion reagent. After adding the M-dilution buffer and the water, the bisulfite stock solution was vortexed periodically over a 10 minute period to dissolve the Zymo CT conversion reagent. After dissolving the Zymo CT conversion reagent, 100 pt of the bisulfite stock solution was removed and added to the bisulfite pre-reaction composition to form a bisulfite reaction composition. The bisulfite reaction composition was incubated for 15 hours at 50° C.

Following the incubation, 300 μl of water was added to the bisulfite reaction composition, and the resulting solution was transferred to the upper chamber of a Microcon 100 filtration device (Millipore), which was centrifuged at 2800 rpm for 18 minutes. The filtrate was removed and 350 pt of water was added to the upper chamber of the filtration device. The filtration device was centrifuged at 2800 rpm for 15 minutes. The filtrate was removed and another 350 μl of water was added to the upper chamber of the filtration device. The filtration device was centrifuged at 2800 rpm for 15 minutes. The filtrate was removed and 350 μl of 0.1 M NaOH was added to the upper chamber of the filtration device. After adding the NaOH, the filtration device was allowed to sit for 5 minutes, and was then centrifuged at 2800 rpm for 15 minutes. The filtrate was removed and 350 μl of water was added to the upper chamber of the filtration device. The filtration device was centrifuged at 2800 rpm for 15 minutes. The filtrate was removed and 50 μl of TE buffer (10 mM Tris Cl; 1 mM EDTA (pH 8.0) (Teknova, T0223)) was added to the upper chamber of the filtration device with gentle mixing. The filtration device was allowed to sit for 5 minutes after which the device was inverted and the TE buffer containing the bisulfite treated DNA was collected.

Preparation of Methylated Genomic DNA

300 ng of methylated genomic DNA (3.3 μl of CpGenome universal methylated DNA (Serologicals P/N S7821)) was added to 44 μl of water and 5 μl of M-dilution buffer (Zymo Research) to form a bisulfite pre-reaction composition. The bisulfite pre-reaction composition was incubated at 37° C. for 15 minutes.

A bisulfite stock solution (approximately 3M bisulfite) was prepared by adding 210 μl of the M-dilution buffer (Zymo Research) and 750 μl of water to a vial provided by Zymo Research (Zymo Research), which contained Zymo CT conversion reagent. After adding the M-dilution buffer and the water, the bisulfite stock solution was vortexed periodically over a 10 minute period to dissolve the Zymo CT conversion reagent. After dissolving the Zymo CT conversion reagent, 100 μl of the bisulfite stock solution was removed and added to the bisulfite pre-reaction composition to form a bisulfite reaction composition. The bisulfite reaction composition was incubated for 15 hours at 50° C.

Following the incubation, 300 μl of water was added to the bisulfite reaction composition, and the resulting solution was transferred to the upper chamber of a Microcon 100 filtration device (Millipore), which was centrifuged at 2800 rpm for 18 minutes. The filtrate was removed and 350 μl of water was added to the upper chamber of the filtration device. The filtration device was centrifuged at 2800 rpm for 15 minutes. The filtrate was removed and another 350 μl of water was added to the upper chamber of the filtration device. The filtration device was centrifuged at 2800 rpm for 15 minutes. The filtrate was removed and 350 μl of 0.1 M NaOH was added to the upper chamber of the filtration device. After adding the NaOH, the filtration device was allowed to sit for 5 minutes, and was then centrifuged at 2800 rpm for 15 minutes. The filtrate was removed and 350 μl of water was added to the upper chamber of the filtration device. The filtration device was centrifuged at 2800 rpm for 15 minutes. The filtrate was removed and 50 μl of TE buffer (10 mM Tris Cl; 1 mM EDTA (pH 8.0) (Teknova, T0223)) was added to the upper chamber of the filtration device with gentle mixing. The filtration device was allowed to sit for 5 minutes after which the device was inverted and the TE buffer containing the bisulfite treated DNA was collected.

PCR Amplification of Bisulfite Treated DNA

Bisulfite treated unmethylated genomic DNA was amplified using the polymerase chain reaction with primers CDH1 Forward Primer (TTTAGTAATTTTAGGTTAGAGGGTTAT (SEQ ID NO.: X)) and CDH1 Reverse Primer (TAACTACAACCAAATAAACCCC (SEQ ID NO.: X)) to generate target DNA from the CDH1 gene (“CDH1 unmethylated target DNA”).

PCR Reaction Composition

| | |
|---|---|
| 2X Taq Gold PCR Master Mix (Applied Biosystems, | 10 μl |
| P/N 4326717) | |
| Aliquot of bisulfite treated unmethylated genomic | 1 μl |
| DNA (5 ng/μl) | |
| CDH1 Forward Primer (5 μM) | 1 μl |
| CDH1 Reverse Primer (5 μM) | 1 μl |
| Water | 7 μl |
| total volume | 20 μl |

PCR reaction compositions were heated to 95° C. for 5 minutes, and were then incubated under thermal cycling conditions. The thermal cycling conditions were 95° C. for 30 seconds, followed by 60° C. for 45 seconds, followed by 72° C. for 1 minute. Those cycling conditions were repeated 40 times. After the PCR reaction compositions were incubated under thermal cycling conditions, those reaction compositions were maintained at 4° C.

Following PCR amplification, an Exo/Sap Solution was made as follows.

Exo/Sap Solution

| | |
|---|---|
| Shrimp Alkaline Phosphatase (SAP) (1 U/μl) | 2 μl |
| (USB, P/N 4326717) | |
| Exonuclease I (10 U/μl) | 0.2 μl |
| Water | 1.8 μl |
| total volume | 4 μl |

The 4 μl Exo/Sap Solution was then added to the 20 μl PCR Reaction Composition to form an Exo/SAP Digestion Composition. The Exo/SAP Digestion Composition was heated to 37° C. for 1 hour, and then heated to 72° C. for 15 minutes.

Bisulfite treated methylated genomic DNA was amplified using the polymerase chain reaction with the CDH1 Forward Primer and the CDH1 Reverse Primer to generate target DNA from the CDH 1 gene (“CDH 1 methylated target DNA”).

PCR Reaction Composition

| | |
|---|---|
| 2X Taq Gold PCR Master Mix (Applied Biosystems, | 10 μl |
| P/N 4326717) | |
| Aliquot of bisulfite treated methylated genomic | 1 μl |
| DNA (5 ng/μl) | |
| CDH1 Forward Primer (5 μM) | 1 μl |
| CDH1 Reverse Primer (5 μM) | 1 μl |
| Water | 7 μl |
| total volume | 20 μl |

PCR reaction compositions were heated to 95° C. for 5 minutes, and were then incubated under thermal cycling conditions. The thermal cycling conditions were 95° C. for 30 seconds, followed by 60° C. for 45 seconds, followed by 72° C. for 1 minute. Those cycling conditions were repeated 40 times. After the PCR reaction compositions were incubated under thermal cycling conditions, those reaction compositions were maintained at 4° C.

Following PCR amplification, an Exo/Sap Solution was made as follows.

Exo/Sap Solution

| | |
|---|---|
| Shrimp Alkaline Phosphatase (SAP) (1 U/μl) | 2 μl |
| (USB, P/N 4326717) | |
| Exonuclease I (10 U/μl) | 0.2 μl |
| Water | 1.8 μl |
| total volume | 4 μl |

The 4 μl Exo/Sap Solution was then added to the 20 μl PCR Reaction Composition to form an Exo/SAP Digestion Composition. The Exo/SAP Digestion Composition was heated to 37° C. for 1 hour, and then heated to 72° C. for 15 minutes.

Following PCR amplification and the Sap/Exo treatment, an equal volume aliquot from the amplification of the CDH1 unmethylated (UnMe) target DNA and the CDH1 methylated (Me) target DNA were mixed to form the “CDH1 template.”

OLA Analysis of the CDH1 Template

The following probes were designed for the following OLA reactions:

| | |
|---|---|
| CDH1 Me P B-OLA: | |
| ACCCGCCCACCCG | (SEQ ID NO.: X) |
| CDH1 Me FAM B-OLA: | |
| (Fam)-CCCCAAAACGAAACTAACG | (SEQ ID NO.: X) |
| CDH1 Blocking Probe: | |
| ACAAAACTAACAACCCACCC | (SEQ ID NO.: X) |
| CDH1 Unme P: | |
| ACCCACCCACCCA | (SEQ ID NO.: X) |
| CDH1 UnMe ASO: | |
| (Fam)-CCCCAAAACAAAACTAACA | (SEQ ID NO.: X) |

The probes CDH1 Me P B-OLA and CDH1 Me FAM B-OLA were a ligation probe set designed to hybridize to the CDH1 methylated target DNA adjacent to each other.

The probes CDH1 UnMe P and CDH1 UnMe ASO were a ligation probe set designed to hybridize to the CDH1 unmethylated target DNA adjacent to each other.

The probe CDH1 Blocking Probe was designed to hybridize to the CDH1 unmethylated target DNA in such a manner that it would compete with the ligation probe sets for binding to the CDH1 unmethylated target DNA.

The probes CDH1 Me P B-OLA, CDH1 Me FAM B-OLA, CDH1 UnMe P, CDH1 UnMe ASO, and CDH1 Blocking Probe were each designed to have a Tm of approximately 60° C.

The following reaction compositions comprising the CDH1 Me P B-OLA and CDH1 Me FAM B-OLA probes were prepared:

Reaction Composition (5 μM Blocking Probe)

| | |
|---|---|
| Taq Ligase buffer 10X | 1 μl |
| Taq Ligase (4 U/μl) | 1 μl |
| CDH1 Template (50:50 UnMe:Me) | 1 μl |
| CDH1 Me FAM B-OLA (0.5 μM) | 1 μl |
| CDH1 Me P B-OLA (0.5 μM) | 1 μl |
| CDH1 Blocking Probe (5 μM) | 1 μl |
| Water | 4 μl |
| total volume | 10 μl |

Reaction Composition (0.5 μM Blocking Probe)

| | |
|---|---|
| Taq Ligase buffer 10X | 1 μl |
| Taq Ligase (4 U/μl) | 1 μl |
| CDH1 Template (50:50 UnMe:Me) | 1 μl |
| CDH1 Me FAM B-OLA (0.5 μM) | 1 μl |
| CDH1 Me P B-OLA (0.5 μM) | 1 μl |
| CDH1 Blocking Probe (0.5 μM) | 1 μl |
| Water | 4 μl |
| total volume | 10 μl |

Reaction Composition (0 μM Blocking Probe)

| | |
|---|---|
| Taq Ligase buffer 10X | 1 μl |
| Taq Ligase (4 U/ul) | 1 μl |
| CDH1 Template (50:50 UnMe:Me) | 1 μl |
| CDH1 Me FAM B-OLA (0.5 μM) | 1 μl |
| CDH1 Me P B-OLA (0.5 μM) | 1 μl |
| CDH1 Blocking Probe (0 μM) | 1 μl |
| Water | 4 μl |
| total volume | 10 μl |

The following reaction compositions comprising the CDH 1 UnMe P and CDH1 UnMe ASO probes were prepared:

Reaction Composition (5 μM Blocking Probe)

| | |
|---|---|
| Taq Ligase buffer 10X | 1 μl |
| Taq Ligase (4 U/μl) | 1 μl |
| CDH1 Template (50:50 UnMe:Me) | 1 μl |
| CDH1 UnMe ASO (0.5 μM) | 1 μl |
| CDH1 UnMe P (0.5 μM) | 1 μl |
| CDH1 Blocking Probe (5 μM) | 1 μl |
| Water | 4 μl |
| total volume | 10 μl |

Reaction Composition (0.5 μM Blocking Probe)

| | |
|---|---|
| Taq Ligase buffer 10X | 1 μl |
| Taq Ligase (4 U/μl) | 1 μl |
| CDH1 Template (50:50 UnMe:Me) | 1 μl |
| CDH1 UnMe ASO (0.5 μM) | 1 μl |
| CDH1 UnMe P (0.5 μM) | 1 μl |
| CDH1 Blocking Probe (0.5 μM) | 1 μl |
| Water | 4 μl |
| total volume | 10 μl |

Reaction Composition (0 μM Blocking Probe)

| | |
|---|---|
| Taq Ligase buffer 10X | 1 μl |
| Taq Ligase (4 U/ul) | 1 μl |
| CDH1 Template (50:50 UnMe:Me) | 1 μl |
| CDH1 UnMe ASO (0.5 μM) | 1 μl |
| CDH1 UnMe P (0.5 μM) | 1 μl |
| CDH1 Blocking Probe (0 μM) | 1 μl |
| Water | 4 μl |
| total volume | 10 μl |

Reaction compositions were incubated under thermal cycling conditions. The thermal cycling conditions were 95° C. for 5 seconds followed by 60° C. for 1 minute. Those cycling conditions were repeated 60 times.

Following incubation under thermal cycling conditions, a 1.2 μl aliquot of the solution was removed and mixed with 12 μl of Hi-Di Formamide (Applied Biosystems). The resulting Formamide solution was heated to 95° C. for five minutes, and the samples were then analyzed by capillary electrophoresis on an ABI PRISM 310 Genetic Analyzer (Applied Biosystems).

### EXAMPLE 3

Preparation of Unmethylated Genomic DNA

300 ng of unmethylated genomic DNA (1 μl of Coriell genomic DNA) was added to 44 μl of water and 5 μl of M-dilution buffer (Zymo Research) to form a bisulfite pre-reaction composition. The bisulfite pre-reaction composition was incubated at 37° C. for 15 minutes.

A bisulfite stock solution (approximately 3M bisulfite) was prepared by adding 210 μl of the M-dilution buffer (Zymo Research) and 750 μl of water to a vial provided by Zymo Research (Zymo Research), which contained Zymo CT conversion reagent. After adding the M-dilution buffer and the water, the bisulfite stock solution was vortexed periodically over a 10 minute period to dissolve the Zymo CT conversion reagent. After dissolving the Zymo CT conversion reagent, 100 μl of the bisulfite stock solution was removed and added to the bisulfite pre-reaction composition to form a bisulfite reaction composition. The bisulfite reaction composition was incubated for 15 hours at 50° C.

Following the incubation, 300 μl of water was added to the bisulfite reaction composition, and the resulting solution was transferred to the upper chamber of a Microcon 100 filtration device (Millipore), which was centrifuged at 2800 rpm for 18 minutes. The filtrate was removed and 350 μl of water was added to the upper chamber of the filtration device. The filtration device was centrifuged at 2800 rpm for 15 minutes. The filtrate was removed and another 350 μl of water was added to the upper chamber of the filtration device. The filtration device was centrifuged at 2800 rpm for 15 minutes. The filtrate was removed and 350 μl of 0.1 M NaOH was added to the upper chamber of the filtration device. After adding the NaOH, the filtration device was allowed to sit for 5 minutes, and was then centrifuged at 2800 rpm for 15 minutes. The filtrate was removed and 350 μl of water was added to the upper chamber of the filtration device. The filtration device was centrifuged at 2800 rpm for 15 minutes. The filtrate was removed and 50 μl of TE buffer (10 mM Tris Cl; 1 mM EDTA (pH 8.0) (Teknova, T0223)) was added to the upper chamber of the filtration device with gentle mixing. The filtration device was allowed to sit for 5 minutes after which the device was inverted and the TE buffer containing the bisulfite treated DNA was collected.

Preparation of Methylated Genomic DNA

300 ng of methylated genomic DNA (3.3 μl of CpGenome universal methylated DNA (Serologicals P/N S7821)) was added to 44 μl of water and 5 μl of M-dilution buffer (Zymo Research) to form a bisulfite pre-reaction composition. The bisulfite pre-reaction composition was incubated at 37° C. for 15 minutes.

A bisulfite stock solution (approximately 3M bisulfite) was prepared by adding 210 μl of the M-dilution buffer (Zymo Research) and 750 μl of water to a vial provided by Zymo Research (Zymo Research), which contained Zymo CT conversion reagent. After adding the M-dilution buffer and the water, the bisulfite stock solution was vortexed periodically over a 10 minute period to dissolve the Zymo CT conversion reagent. After dissolving the Zymo CT conversion reagent, 100 μl of the bisulfite stock solution was removed and added to the bisulfite pre-reaction composition to form a bisulfite reaction composition. The bisulfite reaction composition was incubated for 15 hours at 50° C.

Following the incubation, 300 μl of water was added to the bisulfite reaction composition, and the resulting solution was transferred to the upper chamber of a Microcon 100 filtration device (Millipore), which was centrifuged at 2800 rpm for 18 minutes. The filtrate was removed and 350 μl of water was added to the upper chamber of the filtration device. The filtration device was centrifuged at 2800 rpm for 15 minutes. The filtrate was removed and another 350 μl of water was added to the upper chamber of the filtration device. The filtration device was centrifuged at 2800 rpm for 15 minutes. The filtrate was removed and 350 μl of 0.1 M NaOH was added to the upper chamber of the filtration device. After adding the NaOH, the filtration device was allowed to sit for 5 minutes, and was then centrifuged at 2800 rpm for 15 minutes. The filtrate was removed and 350 μl of water was added to the upper chamber of the filtration device. The filtration device was centrifuged at 2800 rpm for 15 minutes. The filtrate was removed and 50 μl of TE buffer (10 mM Tris Cl; 1 mM EDTA (pH 8.0) (Teknova, T0223)) was added to the upper chamber of the filtration device with gentle mixing. The filtration device was allowed to sit for 5 minutes after which the device was inverted and the TE buffer containing the bisulfite treated DNA was collected.

PCR Amplification of Bisulfite Treated DNA

Bisulfite treated unmethylated genomic DNA was amplified using the polymerase chain reaction with primers BRCA Forward Primer (TTAGAGTAGAGGGTGAAGGTTTTTT (SEQ ID NO.: X)) and BRCA Reverse Primer (AACAAACTAAATAACCAATCCAAAAC (SEQ ID NO.: X)) to generate target DNA from the BRCA gene (“BRCA unmethylated target DNA”) as follows.

PCR Reaction Composition

| | |
|---|---|
| 2X Taq Gold PCR Master Mix (Applied Biosystems, | 10 μl |
| P/N 4326717) | |
| Aliquot of bisulfite treated unmethylated genomic | 1 μl |
| DNA (5 ng/μl) | |
| BRCA Forward Primer (5 μM) | 1 μl |
| BRCA Reverse Primer (5 μM) | 1 μl |
| Water | 7 μl |
| total volume | 20 μl |

PCR reaction compositions were heated to 95° C. for 5 minutes, and were then incubated under thermal cycling conditions. The thermal cycling conditions were 95° C. for 30 seconds, followed by 60° C. for 45 seconds, followed by 72° C. for 1 minute. Those cycling conditions were repeated 40 times. After the PCR reaction compositions were incubated under thermal cycling conditions, those reaction compositions were maintained at 4° C.

Following PCR amplification, an Exo/Sap Solution was made as follows.

Exo/Sap Solution

| | |
|---|---|
| Shrimp Alkaline Phosphatase (SAP) (1 U/μl) | 2 μl |
| (USB, P/N 4326717) | |
| Exonuclease I (10 U/μl) | 0.2 μl |
| Water | 1.8 μl |
| total volume | 4 μl |

The 4 μl Exo/Sap Solution was then added to the 20 μl PCR Reaction Composition to form an Exo/SAP Digestion Composition. The Exo/SAP Digestion Composition was heated to 37° C. for 1 hour, and then heated to 72° C. for 15 minutes.

Bisulfite treated methylated genomic DNA was amplified using the polymerase chain reaction with the BRCA Forward Primer and the BRCA Reverse Primer to generate target DNA from the BRCA gene (“BRCA methylated target DNA”) as follows.

PCR Reaction Composition

| | |
|---|---|
| 2X Taq Gold PCR Master Mix (Applied Biosystems, | 10 μl |
| P/N 4326717) | |
| Aliquot of bisulfite treated methylated genomic | 1 μl |
| DNA (5 ng/μl) | |
| BRCA Forward Primer (5 μM) | 1 μl |
| BRCA Reverse Primer (5 μM) | 1 μl |
| Water | 7 μl |
| total volume | 20 μl |

PCR reaction compositions were heated to 95° C. for 5 minutes, and were then incubated under thermal cycling conditions. The thermal cycling conditions were 95° C. for 30 seconds, followed by 60° C. for 45 seconds, followed by 72° C. for 1 minute. Those cycling conditions were repeated 40 times. After the PCR reaction compositions were incubated under thermal cycling conditions, those reaction compositions were maintained at 4° C.

Following PCR amplification, an Exo/Sap Solution was made as follows.

Exo/Sap Solution

| | |
|---|---|
| Shrimp Alkaline Phosphatase (SAP) (1 U/μl) | 2 μl |
| (USB, P/N 4326717) | |
| Exonuclease I (10 U/μl) | 0.2 μl |
| Water | 1.8 μl |
| total volume | 4 μl |

The 4 μl Exo/Sap Solution was then added to the 20 μl PCR Reaction Composition to form an Exo/SAP Digestion Composition. The Exo/SAP Digestion Composition was heated to 37° C. for 1 hour, and then heated to 72° C. for 15 minutes.

Following PCR amplification and the Sap/Exo treatment, an equal volume aliquot from the amplification of the BRCA unmethylated (UnMe) target DNA and the BRCA methylated (Me) target DNA were mixed to form the “BRCA template.”

OLA Analysis of the BRCA Template

The following probes were designed for the following OLA reactions:

| | |
|---|---|
| BRCA Me P B-OLA: | |
| CGACGTAAACTCGCTAAAAC | (SEQ ID NO.: X) |
| BRCA Me FAM B-OLA: | |
| (FAM)-CAAATAAATTAAAACTACGACTACG | (SEQ ID NO.: X) |
| BRCA Blocking Probe: | |
| ACTACAACTACACAACATAAACTCAC | (SEQ ID NO.: X) |
| BRCA UnMe P: | |
| CAACATAAACTCACTAAAAC | (SEQ ID NO.: X) |
| BRCA UnMe ASO: | |
| (FAM)-CAAATAAATTAAAACTACAACTACA | (SEQ ID NO.: X) |

The probes BRCA Me P B-OLA and BRCA Me FAM B-OLA were a ligation probe set designed to hybridize to the BRCA methylated target DNA adjacent to each other.

The probes BRCA UnMe P and BRCA UnMe ASO were a ligation probe set designed to hybridize to the BRCA unmethylated target DNA adjacent to each other.

The probe BRCA Blocking Probe was designed to hybridize to the BRCA unmethylated target DNA in such a manner that it would compete with the ligation probe sets for binding to the BRCA unmethylated target DNA.

The probes BRCA Me P B-OLA, BRCA Me FAM B-OLA, BRCA UnMe P, BRCA UnMe ASO, and BRCA Blocking Probe were each designed to have a Tm of approximately 60° C.

The following reaction compositions comprising the BRCA Me P B-OLA and BRCA Me FAM B-OLA probes were prepared:

Reaction Composition (5 μM Blocking Probe)

| | |
|---|---|
| Taq Ligase buffer 10X | 1 μl |
| Taq Ligase (4 U/μl) | 1 μl |
| BRCA Template (50:50 UnMe:Me) | 1 μl |
| BRCA Me FAM B-OLA (0.5 μM) | 1 μl |
| BRCA Me P B-OLA (0.5 μM) | 1 μl |
| BRCA Blocking Probe (5 μM) | 1 μl |
| Water | 4 μl |
| total volume | 10 μl |

Reaction Composition (0.5 μM Blocking Probe)

| | |
|---|---|
| Taq Ligase buffer 10X | 1 μl |
| Taq Ligase (4 U/μl) | 1 μl |
| BRCA Template (50:50 UnMe:Me) | 1 μl |
| BRCA Me FAM B-OLA (0.5 μM) | 1 μl |
| BRCA Me P B-OLA (0.5 μM) | 1 μl |
| BRCA Blocking Probe (0.5 μM) | 1 μl |
| Water | 4 μl |
| total volume | 10 μl |

Reaction Composition (0 μM Blocking Probe)

| | |
|---|---|
| Taq Ligase buffer 10X | 1 μl |
| Taq Ligase (4 U/ul) | 1 μl |
| BRCA Template (50:50 UnMe:Me) | 1 μl |
| BRCA Me FAM B-OLA (0.5 μM) | 1 μl |
| BRCA Me P B-OLA (0.5 μM) | 1 μl |
| BRCA Blocking Probe (0 μM) | 1 μl |
| Water | 4 μl |
| total volume | 10 μl |

The following reaction compositions comprising the BRCA UnMe P and BRCA UnMe ASO probes were prepared:

Reaction Composition (5 μM Blocking Probe)

| | |
|---|---|
| Taq Ligase buffer 10X | 1 μl |
| Taq Ligase (4 U/μl) | 1 μl |
| BRCA Template (50:50 UnMe:Me) | 1 μl |
| BRCA UnMe ASO (0.5 μM) | 1 μl |
| BRCA UnMe P (0.5 μM) | 1 μl |
| BRCA Blocking Probe (5 μM) | 1 μl |
| Water | 4 μl |
| total volume | 10 μl |

Reaction Composition (0.5 μM Blocking Probe)

| | |
|---|---|
| Taq Ligase buffer 10X | 1 μl |
| Taq Ligase (4 U/μl) | 1 μl |
| BRCA Template (50:50 UnMe:Me) | 1 μl |
| BRCA UnMe ASO (0.5 μM) | 1 μl |
| BRCA UnMe P (0.5 μM) | 1 μl |
| BRCA Blocking Probe (0.5 μM) | 1 μl |
| Water | 4 μl |
| total volume | 10 μl |

Reaction Composition (0 μM Blocking Probe)

| | |
|---|---|
| Taq Ligase buffer 10X | 1 μl |
| Taq Ligase (4 U/ul) | 1 μl |
| BRCA Template (50:50 UnMe:Me) | 1 μl |
| BRCA UnMe ASO (0.5 μM) | 1 μl |
| BRCA UnMe P (0.5 μM) | 1 μl |
| BRCA Blocking Probe (0 μM) | 1 μl |
| Water | 4 μl |
| total volume | 10 μl |

Reaction compositions were incubated under thermal cycling conditions. The thermal cycling conditions were 95° C. for 5 seconds followed by 60° C. for 1 minute. Those cycling conditions were repeated 60 times.

Following incubation under thermal cycling conditions, a 1.2 μl aliquot of the solution was removed and mixed with 12 μl of Hi-Di Formamide (Applied Biosystems). The resulting Formamide solution was heated to 95° C. for five minutes, and the samples were then analyzed by capillary electrophoresis on an ABI PRISM 310 Genetic Analyzer (Applied Biosystems).

### EXAMPLE 4

Preparation of Unmethylated Genomic DNA

300 ng of unmethylated genomic DNA (1 μl of Coriell genomic DNA) was added to 44 μl of water and 5 μl of M-dilution buffer (Zymo Research) to form a bisulfite pre-reaction composition. The bisulfite pre-reaction composition was incubated at 37° C. for 15 minutes.

A bisulfite stock solution (approximately 3M bisulfite) was prepared by adding 210 μl of the M-dilution buffer (Zymo Research) and 750 μl of water to a vial provided by Zymo Research (Zymo Research), which contained Zymo CT conversion reagent. After adding the M-dilution buffer and the water, the bisulfite stock solution was vortexed periodically over a 10 minute period to dissolve the Zymo CT conversion reagent. After dissolving the Zymo CT conversion reagent, 100 μl of the bisulfite stock solution was removed and added to the bisulfite pre-reaction composition to form a bisulfite reaction composition. The bisulfite reaction composition was incubated for 15 hours at 50° C.

Following the incubation, 300 μl of water was added to the bisulfite reaction composition, and the resulting solution was transferred to the upper chamber of a Microcon 100 filtration device (Millipore), which was centrifuged at 2800 rpm for 18 minutes. The filtrate was removed and 350 μl of water was added to the upper chamber of the filtration device. The filtration device was centrifuged at 2800 rpm for 15 minutes. The filtrate was removed and another 350 μl of water was added to the upper chamber of the filtration device. The filtration device was centrifuged at 2800 rpm for 15 minutes. The filtrate was removed and 350 μl of 0.1 M NaOH was added to the upper chamber of the filtration device. After adding the NaOH, the filtration device was allowed to sit for 5 minutes, and was then centrifuged at 2800 rpm for 15 minutes. The filtrate was removed and 350 μl of water was added to the upper chamber of the filtration device. The filtration device was centrifuged at 2800 rpm for 15 minutes. The filtrate was removed and 50 μl of TE buffer (10 mM Tris Cl; 11 mM EDTA (pH 8.0) (Teknova, T0223)) was added to the upper chamber of the filtration device with gentle mixing. The filtration device was allowed to sit for 5 minutes after which the device was inverted and the TE buffer containing the bisulfite treated DNA was collected.

Preparation of Methylated Genomic DNA

300 ng of methylated genomic DNA (3.3 μl of CpGenome universal methylated DNA (Serologicals P/N S7821)) was added to 44 μl of water and 5 μl of M-dilution buffer (Zymo Research) to form a bisulfite pre-reaction composition. The bisulfite pre-reaction composition was incubated at 37° C. for 15 minutes.

A bisulfite stock solution (approximately 3M bisulfite) was prepared by adding 210 μl of the M-dilution buffer (Zymo Research) and 750 μl of water to a vial provided by Zymo Research (Zymo Research), which contained Zymo CT conversion reagent. After adding the M-dilution buffer and the water, the bisulfite stock solution was vortexed periodically over a 10 minute period to dissolve the Zymo CT conversion reagent. After dissolving the Zymo CT conversion reagent, 100 μl of the bisulfite stock solution was removed and added to the bisulfite pre-reaction composition to form a bisulfite reaction composition. The bisulfite reaction composition was incubated for 15 hours at 50° C.

Following the incubation, 300 μl of water was added to the bisulfite reaction composition, and the resulting solution was transferred to the upper chamber of a Microcon 100 filtration device (Millipore), which was centrifuged at 2800 rpm for 18 minutes. The filtrate was removed and 350 μl of water was added to the upper chamber of the filtration device. The filtration device was centrifuged at 2800 rpm for 15 minutes. The filtrate was removed and another 350 pt of water was added to the upper chamber of the filtration device. The filtration device was centrifuged at 2800 rpm for 15 minutes. The filtrate was removed and 350 μl of 0.1 M NaOH was added to the upper chamber of the filtration device. After adding the NaOH, the filtration device was allowed to sit for 5 minutes, and was then centrifuged at 2800 rpm for 15 minutes. The filtrate was removed and 350 μl of water was added to the upper chamber of the filtration device. The filtration device was centrifuged at 2800 rpm for 15 minutes. The filtrate was removed and 50 μl of TE buffer (10 mM Tris Cl; 1 mM EDTA (pH 8.0) (Teknova, T0223)) was added to the upper chamber of the filtration device with gentle mixing. The filtration device was allowed to sit for 5 minutes after which the device was inverted and the TE buffer containing the bisulfite treated DNA was collected.

PCR Amplification of Bisulfite Treated DNA

Bisulfite treated unmethylated genomic DNA was amplified using the polymerase chain reaction with primers RasSF Forward Primer (TAGTTTAATGAGTTTAGGTTTTTT (SEQ ID NO.: X)) and RasSF Reverse Primer (CTACACCCAAATTTCCATTA (SEQ ID NO.: X)) to generate target DNA from the RasSF gene (“RasSF unmethylated target DNA”) as follows.

PCR Reaction Composition

| | |
|---|---|
| 2X Taq Gold PCR Master Mix (Applied Biosystems, | 10 μl |
| P/N 4326717) | |
| Aliquot of bisulfite treated unmethylated genomic | 1 μl |
| DNA (5 ng/μl) | |
| RasSF Forward Primer (5 μM) | 1 μl |
| RasSF Reverse Primer (5 μM) | 1 μl |
| Water | 7 μl |
| total volume | 20 μl |

PCR reaction compositions were heated to 95° C. for 5 minutes, and were then incubated under thermal cycling conditions. The thermal cycling conditions were 95° C. for 30 seconds, followed by 60° C. for 45 seconds, followed by 72° C. for 1 minute. Those cycling conditions were repeated 40 times. After the PCR reaction compositions were incubated under thermal cycling conditions, those reaction compositions were maintained at 4° C.

Bisulfite treated methylated genomic DNA was amplified using the polymerase chain reaction with the RasSF Forward Primer and the RasSF Reverse Primer to generate target DNA from the RasSF gene (“RasSF methylated target DNA”).

PCR Reaction Composition

| | |
|---|---|
| 2X Taq Gold PCR Master Mix (Applied Biosystems, | 10 μl |
| P/N 4326717) | |
| Aliquot of bisulfite treated methylated genomic | 1 μl |
| DNA (5 ng/μl) | |
| RasSF Forward Primer (5 μM) | 1 μl |
| RasSF Reverse Primer (5 μM) | 1 μl |
| Water | 7 μl |
| total volume | 20 μl |

PCR reaction compositions were heated to 95° C. for 5 minutes, and were then incubated under thermal cycling conditions. The thermal cycling conditions were 95° C. for 30 seconds, followed by 60° C. for 45 seconds, followed by 72° C. for 1 minute. Those cycling conditions were repeated 40 times. After the PCR reaction compositions were incubated under thermal cycling conditions, those reaction compositions were maintained at 4° C.

An equal volume aliquot from the amplification of the RasSF unmethylated (UnMe) target DNA and the RasSF methylated (Me) target DNA were mixed to form the “RasSF template.”

OLA Analysis of the RasSF Template

The following probes were designed for the following OLA reactions:

| | |
|---|---|
| RasSF Me P B-OLA: | |
| ACCCGCGCTTACTAAC | (SEQ ID NO.: X) |
| RasSF Me FAM B-OLA: | |
| (FAM)-CCTATAACCCCGCCCG | (SEQ ID NO.: X) |
| RasSF Blocking Probe: | |
| CCCCACCCAACCCACAC | (SEQ ID NO.: X) |
| RasSF UnMe P: | |
| ACCCACACTTACTAAC | (SEQ ID NO.: X) |
| RasSF UnMe ASO: | |
| (FAM)-CCTATAACCCCACCCA | (SEQ ID NO.: X) |

The probes RasSF Me P B-OLA and RasSF Me FAM B-OLA were a primer set designed to hybridize to the RasSF methylated target DNA adjacent to each other.

The probes RasSF UnMe P and RasSF UnMe ASO were a ligation probe set designed to hybridize to the RasSF unmethylated target DNA adjacent to each other.

The probe RasSF Blocking Probe was designed to hybridize to the RasSF unmethylated target DNA in such a manner that it would compete with the ligation probe sets for binding to the RasSF unmethylated target DNA.

The probes RasSF Me P B-OLA, RasSF Me FAM B-OLA, RasSF UnMe P, RasSF UnMe ASO, and RasSF Blocking Probe were each designed to have a Tm of approximately 60° C.

The following reaction compositions comprising the RasSF Me P B-OLA and RasSF Me FAM B-OLA probes were prepared:

Reaction Composition (5 μM Blocking Probe)

| | |
|---|---|
| Taq Ligase buffer 10X | 1 μl |
| Taq Ligase (4 U/μl) | 1 μl |
| RasSF Template (50:50 UnMe:Me) | 1 μl |
| RasSF Me FAM B-OLA (0.5 μM) | 1 μl |
| RasSF Me P B-OLA (0.5 μM) | 1 μl |
| RasSF Blocking Probe (5 μM) | 1 μl |
| Water | 4 μl |
| total volume | 10 μl |

Reaction Composition (0.5 μM Blocking Probe)

| | |
|---|---|
| Taq Ligase buffer 10X | 1 μl |
| Taq Ligase (4 U/μl) | 1 μl |
| RasSF Template (50:50 UnMe:Me) | 1 μl |
| RasSF Me FAM B-OLA (0.5 μM) | 1 μl |
| RasSF Me P B-OLA (0.5 μM) | 1 μl |
| RasSF Blocking Probe (0.5 μM) | 1 μl |
| Water | 4 μl |
| total volume | 10 μl |

Reaction Composition (0 μM Blocking Probe)

| | |
|---|---|
| Taq Ligase buffer 10X | 1 μl |
| Taq Ligase (4 U/ul) | 1 μl |
| RasSF Template (50:50 UnMe:Me) | 1 μl |
| RasSF Me FAM B-OLA (0.5 μM) | 1 μl |
| RasSF Me P B-OLA (0.5 μM) | 1 μl |
| RasSF Blocking Probe (0 μM) | 1 μl |
| Water | 4 μl |
| total volume | 10 μl |

The following reaction compositions comprising the RasSF UnMe P and RasSF UnMe ASO probes were prepared:

Reaction Composition (5 μM Blocking Probe)

| | |
|---|---|
| Taq Ligase buffer 10X | 1 μl |
| Taq Ligase (4 U/μl) | 1 μl |
| RasSF Template (50:50 UnMe:Me) | 1 μl |
| RasSF UnMe ASO (0.5 μM) | 1 μl |
| RasSF UnMe P (0.5 μM) | 1 μl |
| RasSF Blocking Probe (5 μM) | 1 μl |
| Water | 4 μl |
| total volume | 10 μl |

Reaction Composition (0.5 μM Blocking Probe)

| | |
|---|---|
| Taq Ligase buffer 10X | 1 μl |
| Taq Ligase (4 U/μl) | 1 μl |
| RasSF Template (50:50 UnMe:Me) | 1 μl |
| RasSF UnMe ASO (0.5 μM) | 1 μl |
| RasSF UnMe P (0.5 μM) | 1 μl |
| RasSF Blocking Probe (0.5 μM) | 1 μl |
| Water | 4 μl |
| total volume | 10 μl |

Reaction Composition (0 μM Blocking Probe)

| | |
|---|---|
| Taq Ligase buffer 10X | 1 μl |
| Taq Ligase (4 U/ul) | 1 μl |
| RasSF Template (50:50 UnMe:Me) | 1 μl |
| RasSF UnMe ASO (0.5 μM) | 1 μl |
| RasSF UnMe P (0.5 μM) | 1 μl |
| RasSF Blocking Probe (0 μM) | 1 μl |
| Water | 4 μl |
| total volume | 10 μl |

Reaction compositions were incubated under thermal cycling conditions. The thermal cycling conditions were 95° C. for 5 seconds followed by 60° C. for 1 minute. Those cycling conditions were repeated 60 times.

Following incubation under thermal cycling conditions, a 1.2 μl aliquot of the solution was removed and mixed with 12 μl of Hi-Di Formamide (Applied Biosystems). The resulting Formamide solution was heated to 95° C. for five minutes, and the samples were then analyzed by capillary electrophoresis on an ABI PRISM 310 Genetic Analyzer (Applied Biosystems).

### EXAMPLE 5

Preparation of Unmethylated Genomic DNA

300 ng of unmethylated genomic DNA (1 μl of Coriell genomic DNA) was added to 44 μl of water and 5 μl of M-dilution buffer (Zymo Research) to form a bisulfite pre-reaction composition. The bisulfite pre-reaction composition was incubated at 37° C. for 15 minutes.

A bisulfite stock solution (approximately 3M bisulfite) was prepared by adding 210 μl of the M-dilution buffer (Zymo Research) and 750 μl of water to a vial provided by Zymo Research (Zymo Research), which contained Zymo CT conversion reagent. After adding the M-dilution buffer and the water, the bisulfite stock solution was vortexed periodically over a 10 minute period to dissolve the Zymo CT conversion reagent. After dissolving the Zymo CT conversion reagent, 100 μl of the bisulfite stock solution was removed and added to the bisulfite pre-reaction composition to form a bisulfite reaction composition. The bisulfite reaction composition was incubated for 15 hours at 50° C.

Following the incubation, 300 μl of water was added to the bisulfite reaction composition, and the resulting solution was transferred to the upper chamber of a Microcon 100 filtration device (Millipore), which was centrifuged at 2800 rpm for 18 minutes. The filtrate was removed and 350 μl of water was added to the upper chamber of the filtration device. The filtration device was centrifuged at 2800 rpm for 15 minutes. The filtrate was removed and another 350 μl of water was added to the upper chamber of the filtration device. The filtration device was centrifuged at 2800 rpm for 15 minutes. The filtrate was removed and 350 μl of 0.1 M NaOH was added to the upper chamber of the filtration device. After adding the NaOH, the filtration device was allowed to sit for 5 minutes, and was then centrifuged at 2800 rpm for 15 minutes. The filtrate was removed and 350 μl of water was added to the upper chamber of the filtration device. The filtration device was centrifuged at 2800 rpm for 15 minutes. The filtrate was removed and 50 μl of TE buffer (10 mM Tris Cl; 1 mM EDTA (pH 8.0) (Teknova, T0223)) was added to the upper chamber of the filtration device with gentle mixing. The filtration device was allowed to sit for 5 minutes after which the device was inverted and the TE buffer containing the bisulfite treated DNA was collected.

The bisulfite treated unmethylated genomic DNA was resuspended at a concentration of 10 ng/μl.

Preparation of Methylated Genomic DNA

300 ng of methylated genomic DNA (3.3 μl of CpGenome universal methylated DNA (Serologicals P/N S7821)) was added to 44 μl of water and 5 μl of M-dilution buffer (Zymo Research) to form a bisulfite pre-reaction composition. The bisulfite pre-reaction composition was incubated at 37° C. for 15 minutes;

A bisulfite stock solution (approximately 3M bisulfite) was prepared by adding 210 μl of the M-dilution buffer (Zymo Research) and 750 μl of water to a vial provided by Zymo Research (Zymo Research), which contained Zymo CT conversion reagent. After adding the M-dilution buffer and the water, the bisulfite stock solution was vortexed periodically over a 10 minute period to dissolve the Zymo CT conversion reagent. After dissolving the Zymo CT conversion reagent, 100 μl of the bisulfite stock solution was removed and added to the bisulfite pre-reaction composition to form a bisulfite reaction composition. The bisulfite reaction composition was incubated for 15 hours at 50° C.

Following the incubation, 300 μl of water was added to the bisulfite reaction composition, and the resulting solution was transferred to the upper chamber of a Microcon 100 filtration device (Millipore), which was centrifuged at 2800 rpm for 18 minutes. The filtrate was removed and 350 μl of water was added to the upper chamber of the filtration device. The filtration device was centrifuged at 2800 rpm for 15 minutes. The filtrate was removed and another 350 μl of water was added to the upper chamber of the filtration device. The filtration device was centrifuged at 2800 rpm for 15 minutes. The filtrate was removed and 350 μl of 0.1 M NaOH was added to the upper chamber of the filtration device. After adding the NaOH, the filtration device was allowed to sit for 5 minutes, and was then centrifuged at 2800 rpm for 15 minutes. The filtrate was removed and 350 μl of water was added to the upper chamber of the filtration device. The filtration device was centrifuged at 2800 rpm for 15 minutes. The filtrate was removed and 50 μl of TE buffer (10 mM Tris Cl; 1 mM EDTA (pH 8.0) (Teknova, T0223)) was added to the upper chamber of the filtration device with gentle mixing. The filtration device was allowed to sit for 5 minutes after which the device was inverted and the TE buffer containing the bisulfite treated DNA was collected.

The bisulfite treated methylated genomic DNA was resuspended at a concentration of 10 ng/μt;

OLA-PCR Reactions

Six different target DNA compositions were prepared by mixing bisulfite treated unmethylated genomic DNA with bisulfite treated methylated genomic DNA at six different concentrations. The six ratios of bisulfite treated unmethylated genomic DNA to bisulfite treated methylated genomic DNA are shown below:

Unmethylated (UnMe):Methylated (Me)
1:1
1:0.1
1:0.01
1:0.001
1:0
0:1

The following probes were designed for the following OLA reactions:

| | |
|---|---|
| SRBC ms Me P B-OLA: | (SEQ ID NO.: X) |
| CCGAAAACCTACTAAAAAACC | |
| TTACTCAGGACTCATCGTCGC | |
| SRBC bs Me B-OLA: | |
| CTCGTAGACTGCGTACCGATCCTTCCGCTA | (SEQ ID NO.: X) |
| TCCCGCG | |
| SRBC Blocking Probe: | |
| ACTATCCCACACCAAAAACCTA | (SEQ ID NO.: X) |

The portions of the probes marked by italics are universal primer specific portions.

Six different OLA reaction compositions were prepared as follows:

1:1 OLA Reaction Composition

| | |
|---|---|
| Taq Ligase buffer 10X | 1 μl |
| Taq Ligase (4 U/μl) | 1 μl |
| Target DNA composition (1:1 UnMe:Me) | 1 μl |
| SRBC ms Me P B-OLA (0.05 μM) | 1 μl |
| SRBC bs Me B-OLA (0.05 μM) | 1 μl |
| SRBC Blocking Probe (0.5 μM) | 1 μl |
| Water | 4 μl |
| total volume | 10 μl |

1:0.1 OLA Reaction Composition

| | |
|---|---|
| Taq Ligase buffer 10X | 1 μl |
| Taq Ligase (4 U/μl) | 1 μl |
| Target DNA composition (1:0.1 UnMe:Me) | 1 μl |
| SRBC ms Me P B-OLA (0.05 μM) | 1 μl |
| SRBC bs Me B-OLA (0.05 μM) | 1 μl |
| SRBC Blocking Probe (0.5 μM) | 1 μl |
| Water | 4 μl |
| total volume | 10 μl |

1:0.01 OLA Reaction Composition

| | |
|---|---|
| Taq Ligase buffer 10X | 1 μl |
| Taq Ligase (4 U/μl) | 1 μl |
| Target DNA composition (1:0.01 UnMe:Me) | 1 μl |
| SRBC ms Me P B-OLA (0.05 μM) | 1 μl |
| SRBC bs Me B-OLA (0.05 μM) | 1 μl |
| SRBC Blocking Probe (0.5 μM) | 1 μl |
| Water | 4 μl |
| total volume | 10 μl |

1:0.001 OLA Reaction Composition

| | |
|---|---|
| Taq Ligase buffer 10X | 1 μl |
| Taq Ligase (4 U/μl) | 1 μl |
| Target DNA composition (1:0.001 UnMe:Me) | 1 μl |
| SRBC ms Me P B-OLA (0.05 μM) | 1 μl |
| SRBC bs Me B-OLA (0.05 μM) | 1 μl |
| SRBC Blocking Probe (0.5 μM) | 1 μl |
| Water | 4 μl |
| total volume | 10 μl |

1:0 OLA Reaction Composition

| | |
|---|---|
| Taq Ligase buffer 10X | 1 μl |
| Taq Ligase (4 U/μl) | 1 μl |
| Target DNA composition (1:0 UnMe:Me) | 1 μl |
| SRBC ms Me P B-OLA (0.05 μM) | 1 μl |
| SRBC bs Me B-OLA (0.05 μM) | 1 μl |
| SRBC Blocking Probe (0.5 μM) | 1 μl |
| Water | 4 μl |
| total volume | 10 μl |

0:1 OLA Reaction Composition

| | |
|---|---|
| Taq Ligase buffer 10X | 1 μl |
| Taq Ligase (4 U/μl) | 1 μl |
| Target DNA composition (0:1 UnMe:Me) | 1 μl |
| SRBC ms Me P B-OLA (0.05 μM) | 1 μl |
| SRBC bs Me B-OLA (0.05 μM) | 1 μl |
| SRBC Blocking Probe (0.5 μM) | 1 μl |
| Water | 4 μl |
| total volume | 10 μl |

Reaction compositions were incubated under thermal cycling conditions. The thermal cycling conditions were 95° C. for 5 seconds followed by 60° C. for 1 minute. Those cycling conditions were repeated 90 times.

Following incubation under thermal cycling conditions, a 1 A1 aliquot of each OLA reaction composition was removed and was included in a PCR reaction composition. The primers used in the PCR reactions were: PCR Primer A (5′ CTCGTAGACTGCGTACCGATC (SEQ ID NO.: X)) and PCR Primer B (5′ (FAM)-GCGACGATGAGTCCTGAGTAA (SEQ ID NO.: X)). The PCR reaction compositions were prepared as follows:

1:1 PCR Reaction Composition

| | |
|---|---|
| AmpliTaq Gold Master Mix | 10 μl |
| Aliquot of 1:1 OLA Reaction Composition | 1 μl |
| PCR Primer A (0.5 μM) | 1 μl |
| PCR Primer B (0.5 μM) | 1 μl |
| Water | 7 μl |
| total volume | 20 μl |

1:0.1 PCR Reaction Composition

| | |
|---|---|
| AmpliTaq Gold Master Mix | 10 μl |
| Aliquot of 1:0.1 OLA Reaction Composition | 1 μl |
| PCR Primer A (0.5 μM) | 1 μl |
| PCR Primer B (0.5 μM) | 1 μl |
| Water | 7 μl |
| total volume | 20 μl |

1:0.01 PCR Reaction Composition

| | |
|---|---|
| AmpliTaq Gold Master Mix | 10 μl |
| Aliquot of 1:0.01 OLA Reaction Composition | 1 μl |
| PCR Primer A (0.5 μM) | 1 μl |
| PCR Primer B (0.5 μM) | 1 μl |
| Water | 7 μl |
| total volume | 20 μl |

1:0.001 PCR Reaction Composition

| | |
|---|---|
| AmpliTaq Gold Master Mix | 10 μl |
| Aliquot of 1:0.001 OLA Reaction Composition | 1 μl |
| PCR Primer A (0.5 μM) | 1 μl |
| PCR Primer B (0.5 μM) | 1 μl |
| Water | 7 μl |
| total volume | 20 μl |

1:0 PCR Reaction Composition

| | |
|---|---|
| AmpliTaq Gold Master Mix | 10 μl |
| Aliquot of 1:0 OLA Reaction Composition | 1 μl |
| PCR Primer A (0.5 μM) | 1 μl |
| PCR Primer B (0.5 μM) | 1 μl |
| Water | 7 μl |
| total volume | 20 μl |

0:1 PCR Reaction Composition

| | |
|---|---|
| AmpliTaq Gold Master Mix | 10 μl |
| Aliquot of 0:1 OLA Reaction Composition | 1 μl |
| PCR Primer A (0.5 μM) | 1 μl |
| PCR Primer B (0.5 μM) | 1 μl |
| Water | 7 μl |
| total volume | 20 μl |

PCR reaction compositions were heated to 95° C. for 5 minutes, and were then incubated under thermal cycling conditions. The thermal cycling conditions were 95° C. for 5 seconds followed by 60° C. for 30 seconds. Those cycling conditions were repeated 30 times. After the PCR reaction compositions were incubated under thermal cycling conditions, those reaction compositions were maintained at 4° C.

### EXAMPLE 6

Preparation of Unmethylated Genomic DNA

300 ng of unmethylated genomic DNA (1 μl of Coriell genomic DNA) was added to 44 μl of water and 5 μl of M-dilution buffer (Zymo Research) to form a bisulfite pre-reaction composition. The bisulfite pre-reaction composition was incubated at 37° C. for 15 minutes.

A bisulfite stock solution (approximately 3M bisulfite) was prepared by adding 210 μl of the M-dilution buffer (Zymo Research) and 750 μl of water to a vial provided by Zymo Research (Zymo Research), which contained Zymo CT conversion reagent. After adding the M-dilution buffer and the water, the bisulfite stock solution was vortexed periodically over a 10 minute period to dissolve the Zymo CT conversion reagent. After dissolving the Zymo CT conversion reagent, 100 μl of the bisulfite stock solution was removed and added to the bisulfite pre-reaction composition to form a bisulfite reaction composition. The bisulfite reaction composition was incubated for 15 hours at 50° C.

Following the incubation, 300 μl of water was added to the bisulfite reaction composition, and the resulting solution was transferred to the upper chamber of a Microcon 100 filtration device (Millipore), which was centrifuged at 2800 rpm for 18 minutes. The filtrate was removed and 350 μl of water was added to the upper chamber of the filtration device. The filtration device was centrifuged at 2800 rpm for 15 minutes. The filtrate was removed and another 350 μl of water was added to the upper chamber of the filtration device. The filtration device was centrifuged at 2800 rpm for 15 minutes. The filtrate was removed and 350 μl of 0.1 M NaOH was added to the upper chamber of the filtration device. After adding the NaOH, the filtration device was allowed to sit for 5 minutes, and was then centrifuged at 2800 rpm for 15 minutes. The filtrate was removed and 350 μl of water was added to the upper chamber of the filtration device. The filtration device was centrifuged at 2800 rpm for 15 minutes. The filtrate was removed and 50 μl of TE buffer (10 mM Tris Cl; 1 mM EDTA (pH 8.0) (Teknova, T0223)) was added to the upper chamber of the filtration device with gentle mixing. The filtration device was allowed to sit for 5 minutes after which the device was inverted and the TE buffer containing the bisulfite treated DNA was collected.

The bisulfite treated unmethylated genomic DNA was resuspended at a concentration of 10 ng/μl.

Preparation of Methylated Genomic DNA

300 ng of methylated genomic DNA (3.3 μl of CpGenome universal methylated DNA (Serologicals P/N S7821)) was added to 44 μl of water and 5 μl of M-dilution buffer (Zymo Research) to form a bisulfite pre-reaction composition. The bisulfite pre-reaction composition was incubated at 37° C. for 15 minutes.

A bisulfite stock solution (approximately 3M bisulfite) was prepared by adding 210 μl of the M-dilution buffer (Zymo Research) and 750 μl of water to a vial provided by Zymo Research (Zymo Research), which contained Zymo CT conversion reagent. After adding the M-dilution buffer and the water, the bisulfite stock solution was vortexed periodically over a 10 minute period to dissolve the Zymo CT conversion reagent. After dissolving the Zymo CT conversion reagent, 100 μl of the bisulfite stock solution was removed and added to the bisulfite pre-reaction composition to form a bisulfite reaction composition. The bisulfite reaction composition was incubated for 15 hours at 50° C.

Following the incubation, 300 μl of water was added to the bisulfite reaction composition, and the resulting solution was transferred to the upper chamber of a Microcon 100 filtration device (Millipore), which was centrifuged at 2800 rpm for 18 minutes. The filtrate was removed and 350 μl of water was added to the upper chamber of the filtration device. The filtration device was centrifuged at 2800 rpm for 15 minutes. The filtrate was removed and another 350 μl of water was added to the upper chamber of the filtration device. The filtration device was centrifuged at 2800 rpm for 15 minutes. The filtrate was removed and 350 μl of 0.1 M NaOH was added to the upper chamber of the filtration device. After adding the NaOH, the filtration device was allowed to sit for 5 minutes, and was then centrifuged at 2800 rpm for 15 minutes. The filtrate was removed and 350 μl of water was added to the upper chamber of the filtration device. The filtration device was centrifuged at 2800 rpm for 15 minutes. The filtrate was removed and 50 μl of TE buffer (10 mM Tris Cl; 1 mM EDTA (pH 8.0) (Teknova, T0223)) was added to the upper chamber of the filtration device with gentle mixing. The filtration device was allowed to sit for 5 minutes after which the device was inverted and the TE buffer containing the bisulfite treated DNA was collected.

The bisulfite treated methylated genomic DNA was resuspended at a concentration of 10 ng/μl.

OLA-PCR Reactions

Six different target DNA compositions were prepared by mixing bisulfite treated unmethylated genomic DNA with bisulfite treated methylated genomic DNA at six different concentrations. The six ratios of bisulfite treated unmethylated genomic DNA to bisulfite treated methylated genomic DNA are shown below:

Unmethylated (UnMe):Methylated (Me)
1:1
1:0.1
1:0.01
1:0.001
1:0
0:1

The following probes were designed for the following OLA reactions:

| | |
|---|---|
| P16 ms Me P B-OLA: | |
| CGACCGTAACCAACCAATCA | (SEQ ID NO.: X) |
| TTACTCAGGACTCATCGTCGC | |
| P16 bs Me B-OLA: | |
| CTCGTAGACTGCGTACCGATCCCGAC | (SEQ ID NO.: X) |
| CCCGAACCG | |
| P16 Blocking Probe: | |
| CAACCCCAAACCACAACCAT | (SEQ ID NO.: X) |

The portions of the probes marked by italics are universal primer specific portions.

Six different OLA reaction compositions were prepared as follows:

1:1 OLA Reaction Composition

| | |
|---|---|
| Taq Ligase buffer 10X | 1 μl |
| Taq Ligase (4 U/μl) | 1 μl |
| Target DNA composition (1:1 UnMe:Me) | 1 μl |
| P16 ms Me P B-OLA (0.05 μM) | 1 μl |
| P16 bs Me B-OLA (0.05 μM) | 1 μl |
| P16 Blocking Probe (0.5 μM) | 1 μl |
| Water | 4 μl |
| total volume | 10 μl |

1:0.1 OLA Reaction Composition

| | |
|---|---|
| Taq Ligase buffer 10X | 1 μl |
| Taq Ligase (4 U/μl) | 1 μl |
| Target DNA composition (1:0.1 UnMe:Me) | 1 μl |
| P16 ms Me P B-OLA (0.05 μM) | 1 μl |
| P16 bs Me B-OLA (0.05 μM) | 1 μl |
| P16 Blocking Probe (0.5 μM) | 1 μl |
| Water | 4 μl |
| total volume | 10 μl |

1:0.01 OLA Reaction Composition

| | |
|---|---|
| Taq Ligase buffer 10X | 1 μl |
| Taq Ligase (4 U/μl) | 1 μl |
| Target DNA composition (1:0.01 UnMe:Me) | 1 μl |
| P16 ms Me P B-OLA (0.05 μM) | 1 μl |
| P16 bs Me B-OLA (0.05 μM) | 1 μl |
| P16 Blocking Probe (0.5 μM) | 1 μl |
| Water | 4 μl |
| total volume | 10 μl |

1:0.001 OLA Reaction Composition

| | |
|---|---|
| Taq Ligase buffer 10X | 1 μl |
| Taq Ligase (4 U/μl) | 1 μl |
| Target DNA composition (1:0.001 UnMe:Me) | 1 μl |
| P16 ms Me P B-OLA (0.05 μM) | 1 μl |
| P16 bs Me B-OLA (0.05 μM) | 1 μl |
| P16 Blocking Probe (0.5 μM) | 1 μl |
| Water | 4 μl |
| total volume | 10 μl |

1:0 OLA Reaction Composition

| | |
|---|---|
| Taq Ligase buffer 10X | 1 μl |
| Taq Ligase (4 U/μl) | 1 μl |
| Target DNA composition (1:0 UnMe:Me) | 1 μl |
| P16 ms Me P B-OLA (0.05 μM) | 1 μl |
| P16 bs Me B-OLA (0.05 μM) | 1 μl |
| P16 Blocking Probe (0.5 μM) | 1 μl |
| Water | 4 μl |
| total volume | 10 μl |

0:1 OLA Reaction Composition

| | |
|---|---|
| Taq Ligase buffer 10X | 1 μl |
| Taq Ligase (4 U/μl) | 1 μl |
| Target DNA composition (0:1 UnMe:Me) | 1 μl |
| P16 ms Me P B-OLA (0.05 μM) | 1 μl |
| P16 bs Me B-OLA (0.05 μM) | 1 μl |
| P16 Blocking Probe (0.5 μM) | 1 μl |
| Water | 4 μl |
| total volume | 10 μl |

Reaction compositions were incubated under thermal cycling conditions. The thermal cycling conditions were 95° C. for 5 seconds followed by 60° C. for 1 minute. Those cycling conditions were repeated 90 times.

Following incubation under thermal cycling conditions, a 1 μl aliquot of each OLA reaction composition was removed and was included in a PCR reaction composition. The primers used in the PCR reaction were: PCR Primer A (5′ CTCGTAGACTGCGTACCGATC (SEQ ID NO.: X)) and PCR Primer B (5′ (FAM)-GCGACGATGAGTCCTGAGTAA (SEQ ID NO.: X)). The PCR reaction compositions were prepared as follows:

1:1 PCR Reaction Composition

| | |
|---|---|
| AmpliTaq Gold Master Mix | 10 μl |
| Aliquot of 1:1 OLA Reaction Composition | 1 μl |
| PCR Primer A (0.5 μM) | 1 μl |
| PCR Primer B (0.5 μM) | 1 μl |
| Water | 7 μl |
| total volume | 20 μl |

1:0.1 PCR Reaction Composition

| | |
|---|---|
| AmpliTaq Gold Master Mix | 10 μl |
| Aliquot of 1:0.1 OLA Reaction Composition | 1 μl |
| PCR Primer A (0.5 μM) | 1 μl |
| PCR Primer B (0.5 μM) | 1 μl |
| Water | 7 μl |
| total volume | 20 μl |

1:0.01 PCR Reaction Composition

| | |
|---|---|
| AmpliTaq Gold Master Mix | 10 μl |
| Aliquot of 1:0.01 OLA Reaction Composition | 1 μl |
| PCR Primer A (0.5 μM) | 1 μl |
| PCR Primer B (0.5 μM) | 1 μl |
| Water | 7 μl |
| total volume | 20 μl |

1:0.001 PCR Reaction Composition

| | |
|---|---|
| AmpliTaq Gold Master Mix | 10 μl |
| Aliquot of 1:0.001 OLA Reaction Composition | 1 μl |
| PCR Primer A (0.5 μM) | 1 μl |
| PCR Primer B (0.5 μM) | 1 μl |
| Water | 7 μl |
| total volume | 20 μl |

1:0 PCR Reaction Composition

| | |
|---|---|
| AmpliTaq Gold Master Mix | 10 μl |
| Aliquot of 1:0 OLA Reaction Composition | 1 μl |
| PCR Primer A (0.5 μM) | 1 μl |
| PCR Primer B (0.5 μM) | 1 μl |
| Water | 7 μl |
| total volume | 20 μl |

0:1 PCR Reaction Composition

| | |
|---|---|
| AmpliTaq Gold Master Mix | 10 μl |
| Aliquot of 0:1 OLA Reaction Composition | 1 μl |
| PCR Primer A (0.5 μM) | 1 μl |
| PCR Primer B (0.5 μM) | 1 μl |
| Water | 7 μl |
| total volume | 20 μl |

PCR reaction compositions were heated to 95° C. for 5 minutes, and were then incubated under thermal cycling conditions. The thermal cycling conditions were 95° C. for 5 seconds followed by 60° C. for 30 seconds. Those cycling conditions were repeated 30 times. After the PCR reaction compositions were incubated under thermal cycling conditions, those reaction compositions were maintained at 4° C.

### EXAMPLE 7

Preparation of Unmethylated Genomic DNA

300 ng of unmethylated genomic DNA (1 pt of Coriell genomic DNA) was added to 44 μl of water and 5 μl of M-dilution buffer (Zymo Research) to form a bisulfite pre-reaction composition. The bisulfite pre-reaction composition was incubated at 37° C. for 15 minutes.

A bisulfite stock solution (approximately 3M bisulfite) was prepared by adding 210 μl of the M-dilution buffer (Zymo Research) and 750 μl of water to a vial provided by Zymo Research (Zymo Research), which contained Zymo CT conversion reagent. After adding the M-dilution buffer and the water, the bisulfite stock solution was vortexed periodically over a 10 minute period to dissolve the Zymo CT conversion reagent. After dissolving the Zymo CT conversion reagent, 100 μl of the bisulfite stock solution was removed and added to the bisulfite pre-reaction composition to form a bisulfite reaction composition. The bisulfite reaction composition was incubated for 15 hours at 50° C.

Following the incubation, 300 pt of water was added to the bisulfite reaction composition, and the resulting solution was transferred to the upper chamber of a Microcon 100 filtration device (Millipore), which was centrifuged at 2800 rpm for 18 minutes. The filtrate was removed and 350 μl of water was added to the upper chamber of the filtration device. The filtration device was centrifuged at 2800 rpm for 15 minutes. The filtrate was removed and another 350 μl of water was added to the upper chamber of the filtration device. The filtration device was centrifuged at 2800 rpm for 15 minutes. The filtrate was removed and 350 μl of 0.1 M NaOH was added to the upper chamber of the filtration device. After adding the NaOH, the filtration device was allowed to sit for 5 minutes, and was then centrifuged at 2800 rpm for 15 minutes. The filtrate was removed and 350 μl of water was added to the upper chamber of the filtration device. The filtration device was centrifuged at 2800 rpm for 15 minutes. The filtrate was removed and 50 μl of TE buffer (10 mM Tris Cl; 1 mM EDTA (pH 8.0) (Teknova, T0223)) was added to the upper chamber of the filtration device with gentle mixing. The filtration device was allowed to sit for 5 minutes after which the device was inverted and the TE buffer containing the bisulfite treated DNA was collected.

The bisulfite treated unmethylated genomic DNA was resuspended at a concentration of 16 ng/μl.

Preparation of Methylated Genomic DNA

300 ng of methylated genomic DNA (3.3 μl of CpGenome universal methylated DNA (Serologicals P/N S7821)) was added to 44 μl of water and 5 μl of M-dilution buffer (Zymo Research) to form a bisulfite pre-reaction composition. The bisulfite pre-reaction composition was incubated at 37° C. for 15 minutes.

A bisulfite stock solution (approximately 3M bisulfite) was prepared by adding 210 μl of the M-dilution buffer (Zymo Research) and 750 μl of water to a vial provided by Zymo Research (Zymo Research), which contained Zymo CT conversion reagent. After adding the M-dilution buffer and the water, the bisulfite stock solution was vortexed periodically over a 10 minute period to dissolve the Zymo CT conversion reagent. After dissolving the Zymo CT conversion reagent, 100 μl of the bisulfite stock solution was removed and added to the bisulfite pre-reaction composition to form a bisulfite reaction composition. The bisulfite reaction composition was incubated for 15 hours at 50° C.

Following the incubation, 300 μl of water was added to the bisulfite reaction composition, and the resulting solution was transferred to the upper chamber of a Microcon 100 filtration device (Millipore), which was centrifuged at 2800 rpm for 18 minutes. The filtrate was removed and 350 μl of water was added to the upper chamber of the filtration device. The filtration device was centrifuged at 2800 rpm for 15 minutes. The filtrate was removed and another 350 μl of water was added to the upper chamber of the filtration device. The filtration device was centrifuged at 2800 rpm for 15 minutes. The filtrate was removed and 350 μl of 0.1 M NaOH was added to the upper chamber of the filtration device. After adding the NaOH, the filtration device was allowed to sit for 5 minutes, and was then centrifuged at 2800 rpm for 15 minutes. The filtrate was removed and 350 μl of water was added to the upper chamber of the filtration device. The filtration device was centrifuged at 2800 rpm for 15 minutes. The filtrate was removed and 50 μl of TE buffer (10 mM Tris Cl; 11 mM EDTA (pH 8.0) (Teknova, T0223)) was added to the upper chamber of the filtration device with gentle mixing. The filtration device was allowed to sit for 5 minutes after which the device was inverted and the TE buffer containing the bisulfite treated DNA was collected.

The bisulfite treated methylated genomic DNA was resuspended at a concentration of 10 ng/μl.

OLA-PCR reactions

Six different target DNA compositions were prepared by mixing bisulfite treated unmethylated genomic DNA with bisulfite treated methylated genomic DNA at six different concentrations. The six ratios of bisulfite treated unmethylated genomic DNA to bisulfite treated methylated genomic DNA are shown below:

Unmethylated (UnMe):Methylated (Me)
1:1
1:0.1
1:0.01
1:0.001
1:0
0:1

The following probes were designed for the following OLA reactions:

| | |
|---|---|
| CDH1 ms Me P B-OLA: | |
| ACCCGCCCACCCG | (SEQ ID NO.: X) |
| TTACTCAGGACTCATCGTCGC | |
| CDH1 bs Me B-OLA: | |
| CTCGTAGACTGCGTACCGATCCCCCA | (SEQ ID NO.: X) |
| AAACGAAACTAACG | |
| CDH1 Blocking Probe: | |
| ACAAAACTAACAACCCACCC | (SEQ ID NO.: X) |

The portions of the probes marked by italics are universal primer specific portions.

Six different OLA reaction compositions were prepared as follows:

1:1 OLA Reaction Composition

| | |
|---|---|
| Taq Ligase buffer 10X | 1 μl |
| Taq Ligase (4 U/μl) | 1 μl |
| Target DNA composition (1:1 UnMe:Me) | 1 μl |
| CDH1 ms Me P B-OLA (0.05 μM) | 1 μl |
| CDH1 bs Me B-OLA (0.05 μM) | 1 μl |
| CDH1 Blocking Probe (0.5 μM) | 1 μl |
| Water | 4 μl |
| total volume | 10 μl |

1:0.1 OLA Reaction Composition

| | |
|---|---|
| Taq Ligase buffer 10X | 1 μl |
| Taq Ligase (4 U/μl) | 1 μl |
| Target DNA composition (1:0.1 UnMe:Me) | 1 μl |
| CDH1 ms Me P B-OLA (0.05 μM) | 1 μl |
| CDH1 bs Me B-OLA (0.05 μM) | 1 μl |
| CDH1 Blocking Probe (0.5 μM) | 1 μl |
| Water | 4 μl |
| total volume | 10 μl |

1:0.01 OLA Reaction Composition

| | |
|---|---|
| Taq Ligase buffer 10X | 1 μl |
| Taq Ligase (4 U/μl) | 1 μl |
| Target DNA composition (1:0.01 UnMe:Me) | 1 μl |
| CDH1 ms Me P B-OLA (0.05 μM) | 1 μl |
| CDH1 bs Me B-OLA (0.05 μM) | 1 μl |
| CDH1 Blocking Probe (0.5 μM) | 1 μl |
| Water | 4 μl |
| total volume | 10 μl |

1:0.001 OLA Reaction Composition

| | |
|---|---|
| Taq Ligase buffer 10X | 1 μl |
| Taq Ligase (4 U/μl) | 1 μl |
| Target DNA composition (1:0.001 UnMe:Me) | 1 μl |
| CDH1 ms Me P B-OLA (0.05 μM) | 1 μl |
| CDH1 bs Me B-OLA (0.05 μM) | 1 μl |
| CDH1 Blocking Probe (0.5 μM) | 1 μl |
| Water | 4 μl |
| total volume | 10 μl |

1:0 OLA Reaction Composition

| | |
|---|---|
| Taq Ligase buffer 10X | 1 μl |
| Taq Ligase (4 U/μl) | 1 μl |
| Target DNA composition (1:0 UnMe:Me) | 1 μl |
| CDH1 ms Me P B-OLA (0.05 μM) | 1 μl |
| CDH1 bs Me B-OLA (0.05 μM) | 1 μl |
| CDH1 Blocking Probe (0.5 μM) | 1 μl |
| Water | 4 μl |
| total volume | 10 μl |

0:1 OLA Reaction Composition

| | |
|---|---|
| Taq Ligase buffer 10X | 1 μl |
| Taq Ligase (4 U/μl) | 1 μl |
| Target DNA composition (0:1 UnMe:Me) | 1 μl |
| CDH1 ms Me P B-OLA (0.05 μM) | 1 μl |
| CDH1 bs Me B-OLA (0.05 μM) | 1 μl |
| CDH1 Blocking Probe (0.5 μM) | 1 μl |
| Water | 4 μl |
| total volume | 10 μl |

Reaction compositions were incubated under thermal cycling conditions. The thermal cycling conditions were 95° C. for 5 seconds followed by 60° C. for 1 minute. Those cycling conditions were repeated 90 times.

Following incubation under thermal cycling conditions, a 1 μl aliquot of each OLA reaction composition was removed and was included in a PCR amplification reaction composition. The primers used in the PCR reaction were: PCR Primer A (5′ CTCGTAGACTGCGTACCGATC (SEQ ID NO.: X)) and PCR Primer B (5′ (FAM)-GCGACGATGAGTCCTGAGTAA (SEQ ID NO.: X)). The PCR reaction compositions were prepared as follows:

1:1 PCR Reaction Composition

| | |
|---|---|
| AmpliTaq Gold Master Mix | 10 μl |
| Aliquot of 1:1 OLA Reaction Composition | 1 μl |
| PCR Primer A (0.5 μM) | 1 μl |
| PCR Primer B (0.5 μM) | 1 μl |
| Water | 7 μl |
| total volume | 20 μl |

1:0.1 PCR Reaction Composition

| | |
|---|---|
| AmpliTaq Gold Master Mix | 10 μl |
| Aliquot of 1:0.1 OLA Reaction Composition | 1 μl |
| PCR Primer A (0.5 μM) | 1 μl |
| PCR Primer B (0.5 μM) | 1 μl |
| Water | 7 μl |
| total volume | 20 μl |

1:0.01 PCR Reaction Composition

| | |
|---|---|
| AmpliTaq Gold Master Mix | 10 μl |
| Aliquot of 1:0.01 OLA Reaction Composition | 1 μl |
| PCR Primer A (0.5 μM) | 1 μl |
| PCR Primer B (0.5 μM) | 1 μl |
| Water | 7 μl |
| total volume | 20 μl |

1:0.001 PCR Reaction Composition

| | |
|---|---|
| AmpliTaq Gold Master Mix | 10 μl |
| Aliquot of 1:0.001 OLA Reaction Composition | 1 μl |
| PCR Primer A (0.5 μM) | 1 μl |
| PCR Primer B (0.5 μM) | 1 μl |
| Water | 7 μl |
| total volume | 20 μl |

1:0 PCR Reaction Composition

| | |
|---|---|
| AmpliTaq Gold Master Mix | 10 μl |
| Aliquot of 1:0 OLA Reaction Composition | 1 μl |
| PCR Primer A (0.5 μM) | 1 μl |
| PCR Primer B (0.5 μM) | 1 μl |
| Water | 7 μl |
| total volume | 20 μl |

0:1 PCR Reaction Composition

| | |
|---|---|
| AmpliTaq Gold Master Mix | 10 μl |
| Aliquot of 0:1 OLA Reaction Composition | 1 μl |
| PCR Primer A (0.5 μM) | 1 μl |
| PCR Primer B (0.5 μM) | 1 μl |
| Water | 7 μl |
| total volume | 20 μl |

PCR reaction compositions were heated to 95° C. for 5 minutes, and were then incubated under thermal cycling conditions. The thermal cycling conditions were 95° C. for 5 seconds followed by 60° C. for 30 seconds. Those cycling conditions were repeated 30 times. After the PCR reaction compositions were incubated under thermal cycling conditions, those reaction compositions were maintained at 4° C.

### EXAMPLE 8

Preparation of Unmethylated Genomic DNA

300 ng of unmethylated genomic DNA (1 μl of Coriell genomic DNA) was added to 44 μl of water and 5 μl of M-dilution buffer (Zymo Research) to form a bisulfite pre-reaction composition. The bisulfite pre-reaction composition was incubated at 37° C. for 15 minutes.

A bisulfite stock solution (approximately 3M bisulfite) was prepared by adding 210 μl of the M-dilution buffer (Zymo Research) and 750 μl of water to a vial provided by Zymo Research (Zymo Research), which contained Zymo CT conversion reagent. After adding the M-dilution buffer and the water, the bisulfite stock solution was vortexed periodically over a 10 minute period to dissolve the Zymo CT conversion reagent. After dissolving the Zymo CT conversion reagent, 100 μl of the bisulfite stock solution was removed and added to the bisulfite pre-reaction composition to form a bisulfite reaction composition. The bisulfite reaction composition was incubated for 15 hours at 50° C.

Following the incubation, 300 μl of water was added to the bisulfite reaction composition, and the resulting solution was transferred to the upper chamber of a Microcon 100 filtration device (Millipore), which was centrifuged at 2800 rpm for 18 minutes. The filtrate was removed and 350 μl of water was added to the upper chamber of the filtration device. The filtration device was centrifuged at 2800 rpm for 15 minutes. The filtrate was removed and another 350 μl of water was added to the upper chamber of the filtration device. The filtration device was centrifuged at 2800 rpm for 15 minutes. The filtrate was removed and 350 μl of 0.1 M NaOH was added to the upper chamber of the filtration device. After adding the NaOH, the filtration device was allowed to sit for 5 minutes, and was then centrifuged at 2800 rpm for 15 minutes. The filtrate was removed and 350 μl of water was added to the upper chamber of the filtration device. The filtration device was centrifuged at 2800 rpm for 15 minutes. The filtrate was removed and 50 μl of TE buffer (10 mM Tris Cl; 1 mM EDTA (pH 8.0) (Teknova, T0223)) was added to the upper chamber of the filtration device with gentle mixing. The filtration device was allowed to sit for 5 minutes after which the device was inverted and the TE buffer containing the bisulfite treated DNA was collected.

The bisulfite treated unmethylated genomic DNA was resuspended at a concentration of 10 ng/μl.

Preparation of Methylated Genomic DNA

300 ng of methylated genomic DNA (3.3 μl of CpGenome universal methylated DNA (Serologicals P/N S7821)) was added to 44 μl of water and 5 μl of M-dilution buffer (Zymo Research) to form a bisulfite pre-reaction composition. The bisulfite pre-reaction composition was incubated at 37° C. for 15 minutes.

A bisulfite stock solution (approximately 3M bisulfite) was prepared by adding 210 μl of the M-dilution buffer (Zymo Research) and 750 μl of water to a vial provided by Zymo Research (Zymo Research), which contained Zymo CT conversion reagent. After adding the M-dilution buffer and the water, the bisulfite stock solution was vortexed periodically over a 10 minute period to dissolve the Zymo CT conversion reagent. After dissolving the Zymo CT conversion reagent, 100 μl of the bisulfite stock solution was removed and added to the bisulfite pre-reaction composition to form a bisulfite reaction composition. The bisulfite reaction composition was incubated for 15 hours at 50° C.

Following the incubation, 300 μl of water was added to the bisulfite reaction composition, and the resulting solution was transferred to the upper chamber of a Microcon 100 filtration device (Millipore), which was centrifuged at 2800 rpm for 18 minutes. The filtrate was removed and 350 μl of water was added to the upper chamber of the filtration device. The filtration device was centrifuged at 2800 rpm for 15 minutes. The filtrate was removed and another 350 μl of water was added to the upper chamber of the filtration device. The filtration device was centrifuged at 2800 rpm for 15 minutes. The filtrate was removed and 350 μl of 0.1 M NaOH was added to the upper chamber of the filtration device. After adding the NaOH, the filtration device was allowed to sit for 5 minutes, and was then centrifuged at 2800 rpm for 15 minutes. The filtrate was removed and 350 μl of water was added to the upper chamber of the filtration device. The filtration device was centrifuged at 2800 rpm for 15 minutes. The filtrate was removed and 50 μl of TE buffer (10 mM Tris Cl; 1 mM EDTA (pH 8.0) (Teknova, T0223)) was added to the upper chamber of the filtration device with gentle mixing. The filtration device was allowed to sit for 5 minutes after which the device was inverted and the TE buffer containing the bisulfite treated DNA was collected.

The bisulfite treated methylated genomic DNA was resuspended at a concentration of 10 ng/μl.

OLA-PCR reactions

Six different target DNA compositions were prepared by mixing bisulfite treated unmethylated genomic DNA with bisulfite treated methylated genomic DNA at six different concentrations. The six ratios of bisulfite treated unmethylated genomic DNA to bisulfite treated methylated genomic DNA are shown below:

Unmethylated (UnMe):Methylated (Me)
1:1
1:0.1
1:0.01
1:0.001
1:0
0:1

The following probes were designed for the following OLA reactions:

| | |
|---|---|
| BRCA ms Me P B-OLA: | |
| CGACGTAAACTCGCTAAAAC | (SEQ ID NO.: X) |
| TTACTCAGGACTCATCGTCGC | |
| BRCA bs Me B-OLA: | |
| CTCGTAGACTGCGTACCGATCCAAATAAA | (SEQ ID NO.: X) |
| TTAAAACTACGACTACG | |
| BRCA Blocking Probe: | |
| ACTACAACTACACAACATAAACTCAC | (SEQ ID NO.: X) |

The portions of the probes marked by italics are universal primer specific portions.

Six different OLA reaction compositions were prepared as follows:

1:1 OLA Reaction Composition

| | |
|---|---|
| Taq Ligase buffer 10X | 1 μl |
| Taq Ligase (4 U/μl) | 1 μl |
| Target DNA composition (1:1 UnMe:Me) | 1 μl |
| BRCA ms Me P B-OLA (0.05 μM) | 1 μl |
| BRCA bs Me B-OLA (0.05 μM) | 1 μl |
| BRCA Blocking Probe (0.5 μM) | 1 μl |
| Water | 4 μl |
| total volume | 10 μl |

1:0.1 OLA Reaction Composition

| | |
|---|---|
| Taq Ligase buffer 10X | 1 μl |
| Taq Ligase (4 U/μl) | 1 μl |
| Target DNA composition (1:0.1 UnMe:Me) | 1 μl |
| BRCA ms Me P B-OLA (0.05 μM) | 1 μl |
| BRCA bs Me B-OLA (0.05 μM) | 1 μl |
| BRCA Blocking Probe (0.5 μM) | 1 μl |
| Water | 4 μl |
| total volume | 10 μl |

1:0.01 OLA Reaction Composition

| | |
|---|---|
| Taq Ligase buffer 10X | 1 μl |
| Taq Ligase (4 U/μl) | 1 μl |
| Target DNA composition (1:0.01 UnMe:Me) | 1 μl |
| BRCA ms Me P B-OLA (0.05 μM) | 1 μl |
| BRCA bs Me B-OLA (0.05 μM) | 1 μl |
| BRCA Blocking Probe (0.5 μM) | 1 μl |
| Water | 4 μl |
| total volume | 10 μl |

1:0.001 OLA Reaction Composition

| | |
|---|---|
| Taq Ligase buffer 10X | 1 μl |
| Taq Ligase (4 U/μl) | 1 μl |
| Target DNA composition (1:0.001 UnMe:Me) | 1 μl |
| BRCA ms Me P B-OLA (0.05 μM) | 1 μl |
| BRCA bs Me B-OLA (0.05 μM) | 1 μl |
| BRCA Blocking Probe (0.5 μM) | 1 μl |
| Water | 4 μl |
| total volume | 10 μl |

1:0 OLA Reaction Composition

| | |
|---|---|
| Taq Ligase buffer 10X | 1 μl |
| Taq Ligase (4 U/μl) | 1 μl |
| Target DNA composition (1:0 UnMe:Me) | 1 μl |
| BRCA ms Me P B-OLA (0.05 μM) | 1 μl |
| BRCA bs Me B-OLA (0.05 μM) | 1 μl |
| BRCA Blocking Probe (0.5 μM) | 1 μl |
| Water | 4 μl |
| total volume | 10 μl |

0:1 OLA Reaction Composition

| | |
|---|---|
| Taq Ligase buffer 10X | 1 μl |
| Taq Ligase (4 U/μl) | 1 μl |
| Target DNA composition (0:1 UnMe:Me) | 1 μl |
| BRCA ms Me P B-OLA (0.05 μM) | 1 μl |
| BRCA bs Me B-OLA (0.05 μM) | 1 μl |
| BRCA Blocking Probe (0.5 μM) | 1 μl |
| Water | 4 μl |
| total volume | 10 μl |

Reaction compositions were incubated under thermal cycling conditions. The thermal cycling conditions were 95° C. for 5 seconds followed by 60° C. for 1 minute. Those cycling conditions were repeated 90 times.

Following incubation under thermal cycling conditions, a 1 μl aliquot of each OLA reaction composition was removed and was included in a PCR amplification reaction composition. The primers used in the PCR reaction were: PCR Primer A (5′ CTCGTAGACTGCGTACCGATC (SEQ ID NO.: X)) and PCR Primer B (5′ (FAM)-GCGACGATGAGTCCTGAGTAA (SEQ ID NO.: X)). The PCR reaction compositions were prepared as follows:

1:1 PCR Reaction Composition

| | |
|---|---|
| AmpliTaq Gold Master Mix | 10 μl |
| Aliquot of 1:1 OLA Reaction Composition | 1 μl |
| PCR Primer A (0.5 μM) | 1 μl |
| PCR Primer B (0.5 μM) | 1 μl |
| Water | 7 μl |
| total volume | 20 μl |

1:0.1 PCR Reaction Composition

| | |
|---|---|
| AmpliTaq Gold Master Mix | 10 μl |
| Aliquot of 1:0.1 OLA Reaction Composition | 1 μl |
| PCR Primer A (0.5 μM) | 1 μl |
| PCR Primer B (0.5 μM) | 1 μl |
| Water | 7 μl |
| total volume | 20 μl |

1:0.01 PCR Reaction Composition

| | |
|---|---|
| AmpliTaq Gold Master Mix | 10 μl |
| Aliquot of 1:0.01 OLA Reaction Composition | 1 μl |
| PCR Primer A (0.5 μM) | 1 μl |
| PCR Primer B (0.5 μM) | 1 μl |
| Water | 7 μl |
| total volume | 20 μl |

1:0.001 PCR Reaction Composition

| | |
|---|---|
| AmpliTaq Gold Master Mix | 10 μl |
| Aliquot of 1:0.001 OLA Reaction Composition | 1 μl |
| PCR Primer A (0.5 μM) | 1 μl |
| PCR Primer B (0.5 μM) | 1 μl |
| Water | 7 μl |
| total volume | 20 μl |

1:0 PCR Reaction Composition

| | |
|---|---|
| AmpliTaq Gold Master Mix | 10 μl |
| Aliquot of 1:0 OLA Reaction Composition | 1 μl |
| PCR Primer A (0.5 μM) | 1 μl |
| PCR Primer B (0.5 μM) | 1 μl |
| Water | 7 μl |
| total volume | 20 μl |

0:1 PCR Reaction Composition

| | |
|---|---|
| AmpliTaq Gold Master Mix | 10 μl |
| Aliquot of 0:1 OLA Reaction Composition | 1 μl |
| PCR Primer A (0.5 μM) | 1 μl |
| PCR Primer B (0.5 μM) | 1 μl |
| Water | 7 μl |
| total volume | 20 μl |

PCR reaction compositions were heated to 95° C. for 5 minutes, and were then incubated under thermal cycling conditions. The thermal cycling conditions were 95° C. for 5 seconds followed by 60° C. for 30 seconds. Those cycling conditions were repeated 30 times. After the PCR reaction compositions were incubated under thermal cycling conditions, those reaction compositions were maintained at 4° C.

### EXAMPLE 9

Preparation of Unmethylated Genomic DNA

300 ng of unmethylated genomic DNA (1 μl of Coriell genomic DNA) was added to 44 μl of water and 5 μl of M-dilution buffer (Zymo Research) to form a bisulfite pre-reaction composition. The bisulfite pre-reaction composition was incubated at 37° C. for 15 minutes.

A bisulfite stock solution (approximately 3M bisulfite) was prepared by adding 210 μl of the M-dilution buffer (Zymo Research) and 750 μl of water to a vial provided by Zymo Research (Zymo Research), which contained Zymo CT conversion reagent. After adding the M-dilution buffer and the water, the bisulfite stock solution was vortexed periodically over a 10 minute period to dissolve the Zymo CT conversion reagent. After dissolving the Zymo CT conversion reagent, 100 μl of the bisulfite stock solution was removed and added to the bisulfite pre-reaction composition to form a bisulfite reaction composition. The bisulfite reaction composition was incubated for 15 hours at 50° C.

Following the incubation, 300 μl of water was added to the bisulfite reaction composition, and the resulting solution was transferred to the upper chamber of a Microcon 100 filtration device (Millipore), which was centrifuged at 2800 rpm for 18 minutes. The filtrate was removed and 350 μl of water was added to the upper chamber of the filtration device. The filtration device was centrifuged at 2800 rpm for 15 minutes. The filtrate was removed and another 350 μl of water was added to the upper chamber of the filtration device. The filtration device was centrifuged at 2800 rpm for 15 minutes. The filtrate was removed and 350 μl of 0.1 M NaOH was added to the upper chamber of the filtration device. After adding the NaOH, the filtration device was allowed to sit for 5 minutes, and was then centrifuged at 2800 rpm for 15 minutes. The filtrate was removed and 350 μl of water was added to the upper chamber of the filtration device. The filtration device was centrifuged at 2800 rpm for 15 minutes. The filtrate was removed and 50 μl of TE buffer (10 mM Tris Cl; 1 mM EDTA (pH 8.0) (Teknova, T0223)) was added to the upper chamber of the filtration device with gentle mixing. The filtration device was allowed to sit for 5 minutes after which the device was inverted and the TE buffer containing the bisulfite treated DNA was collected.

The bisulfite treated unmethylated genomic DNA was resuspended at a concentration of 10 ng/μl.

Preparation of Methylated Genomic DNA

300 ng of methylated genomic DNA (3.3 μl of CpGenome universal methylated DNA (Serologicals P/N S7821)) was added to 44 μl of water and 5 μl of M-dilution buffer (Zymo Research) to form a bisulfite pre-reaction composition. The bisulfite pre-reaction composition was incubated at 37° C. for 15 minutes.

A bisulfite stock solution (approximately 3M bisulfite) was prepared by adding 210 μl of the M-dilution buffer (Zymo Research) and 750 μl of water to a vial provided by Zymo Research (Zymo Research), which contained Zymo CT conversion reagent. After adding the M-dilution buffer and the water, the bisulfite stock solution was vortexed periodically over a 10 minute period to dissolve the Zymo CT conversion reagent. After dissolving the Zymo CT conversion reagent, 100 μl of the bisulfite stock solution was removed and added to the bisulfite pre-reaction composition to form a bisulfite reaction composition. The bisulfite reaction composition was incubated for 15 hours at 50° C.

Following the incubation, 300 μl of water was added to the bisulfite reaction composition, and the resulting solution was transferred to the upper chamber of a Microcon 100 filtration device (Millipore), which was centrifuged at 2800 rpm for 18 minutes. The filtrate was removed and 350 μl of water was added to the upper chamber of the filtration device. The filtration device was centrifuged at 2800 rpm for 15 minutes. The filtrate was removed and another 350 μl of water was added to the upper chamber of the filtration device. The filtration device was centrifuged at 2800 rpm for 15 minutes. The filtrate was removed and 350 μl of 0.1 M NaOH was added to the upper chamber of the filtration device. After adding the NaOH, the filtration device was allowed to sit for 5 minutes, and was then centrifuged at 2800 rpm for 15 minutes. The filtrate was removed and 350 μl of water was added to the upper chamber of the filtration device. The filtration device was centrifuged at 2800 rpm for 15 minutes. The filtrate was removed and 50 μl of TE buffer (10 mM Tris Cl; 1 mM EDTA (pH 8.0) (Teknova, T0223)) was added to the upper chamber of the filtration device with gentle mixing. The filtration device was allowed to sit for 5 minutes after which the device was inverted and the TE buffer containing the bisulfite treated DNA was collected.

The bisulfite treated methylated genomic DNA was resuspended at a concentration of 10 ng/μl.

OLA-PCR reactions

Six different target DNA compositions were prepared by mixing bisulfite treated unmethylated genomic DNA with bisulfite treated methylated genomic DNA at six different concentrations. The six ratios of bisulfite treated unmethylated genomic DNA to bisulfite treated methylated genomic DNA are shown below:

Unmethylated (UnMe):Methylated (Me)
1:1
1:0.1
1:0.01
1:0.001
1:0
0:1

The following probes were designed for the following OLA reactions:

| | |
|---|---|
| P15 ms Me P B-OLA: | |
| CGACGCTAACCAAACCC | (SEQ ID NO.: X) |
| TTACTCAGGACTCATCGTCGC | |
| P15 bs Me B-OLA: | |
| CTCGTAGACTGCGTACCGATCCTAATCC | (SEQ ID NO.: X) |
| CCGCGCCG | |
| P15 Blocking Probe: | |
| CCCACACCACAACACTAACC | (SEQ ID NO.: X) |

The portions of the probes marked by italics are universal primer specific portions.

Six different OLA reaction compositions were prepared as follows:

1:1 OLA Reaction Composition

| | |
|---|---|
| Taq Ligase buffer 10× | 1 μl |
| Taq Ligase (4 U/μl) | 1 μl |
| Target DNA composition (1:1 UnMe:Me) | 1 μl |
| P15 ms Me P B-OLA (0.05 μM) | 1 μl |
| P15 bs Me B-OLA (0.05 μM) | 1 μl |
| P15 Blocking Probe (0.5 μM) | 1 μl |
| Water | 4 μl |
| total volume | 10 μl |

1:0.1 OLA Reaction Composition

| | |
|---|---|
| Taq Ligase buffer 10× | 1 μl |
| Taq Ligase (4 U/μl) | 1 μl |
| Target DNA composition (1:0.1 UnMe:Me) | 1 μl |
| P15 ms Me P B-OLA (0.05 μM) | 1 μl |
| P15 bs Me B-OLA (0.05 μM) | 1 μl |
| P15 Blocking Probe (0.5 μM) | 1 μl |
| Water | 4 μl |
| total volume | 10 μl |

1:0.01 OLA Reaction Composition

| | |
|---|---|
| Taq Ligase buffer 10× | 1 μl |
| Taq Ligase (4 U/μl) | 1 μl |
| Target DNA composition (1:0.01 UnMe:Me) | 1 μl |
| P15 ms Me P B-OLA (0.05 μM) | 1 μl |
| P15 bs Me B-OLA (0.05 μM) | 1 μl |
| P15 Blocking Probe (0.5 μM) | 1 μl |
| Water | 4 μl |
| total volume | 10 μl |

1:0.001 OLA Reaction Composition

| | |
|---|---|
| Taq Ligase buffer 10× | 1 μl |
| Taq Ligase (4 U/μl) | 1 μl |
| Target DNA composition (1:0.001 UnMe:Me) | 1 μl |
| P15 ms Me P B-OLA (0.05 μM) | 1 μl |
| P15 bs Me B-OLA (0.05 μM) | 1 μl |
| P15 Blocking Probe (0.5 μM) | 1 μl |
| Water | 4 μl |
| total volume | 10 μl |

1:0 OLA Reaction Composition

| | |
|---|---|
| Taq Ligase buffer 10× | 1 μl |
| Taq Ligase (4 U/μl) | 1 μl |
| Target DNA composition (1:0 UnMe:Me) | 1 μl |
| P15 ms Me P B-OLA (0.05 μM) | 1 μl |
| P15 bs Me B-OLA (0.05 μM) | 1 μl |
| P15 Blocking Probe (0.5 μM) | 1 μl |
| Water | 4 μl |
| total volume | 10 μl |

0:1 OLA Reaction Composition

| | |
|---|---|
| Taq Ligase buffer 10× | 1 μl |
| Taq Ligase (4 U/μl) | 1 μl |
| Target DNA composition (0:1 UnMe:Me) | 1 μl |
| P15 ms Me P B-OLA (0.05 μM) | 1 μl |
| P15 bs Me B-OLA (0.05 μM) | 1 μl |
| P15 Blocking Probe (0.5 μM) | 1 μl |
| Water | 4 μl |
| total volume | 10 μl |

Reaction compositions were incubated under thermal cycling conditions. The thermal cycling conditions were 95° C. for 5 seconds followed by 60° C. for 1 minute. Those cycling conditions were repeated 90 times.

Following incubation under thermal cycling conditions, a 1 μl aliquot of each OLA reaction composition was removed and was included in a PCR amplification reaction. The primers used in the PCR reaction were: PCR Primer A (5′ CTCGTAGACTGCGTACCGATC (SEQ ID NO.: X)) and PCR Primer B (5′ (FAM)-GCGACGATGAGTCCTGAGTAA (SEQ ID NO.: X)). The PCR reaction compositions were prepared as follows:

1:1 PCR Reaction Composition

| | |
|---|---|
| AmpliTaq Gold Master Mix | 10 μl |
| Aliquot of 1:1 OLA Reaction Composition | 1 μl |
| PCR Primer A (0.5 μM) | 1 μl |
| PCR Primer B (0.5 μM) | 1 μl |
| Water | 7 μl |
| total volume | 20 μl |

1:0.1 PCR Reaction Composition

| | |
|---|---|
| AmpliTaq Gold Master Mix | 10 μl |
| Aliquot of 1:0.1 OLA Reaction Composition | 1 μl |
| PCR Primer A (0.5 μM) | 1 μl |
| PCR Primer B (0.5 μM) | 1 μl |
| Water | 7 μl |
| total volume | 20 μl |

1:0.01 PCR Reaction Composition

| | |
|---|---|
| AmpliTaq Gold Master Mix | 10 μl |
| Aliquot of 1:0.01 OLA Reaction Composition | 1 μl |
| PCR Primer A (0.5 μM) | 1 μl |
| PCR Primer B (0.5 μM) | 1 μl |
| Water | 7 μl |
| total volume | 20 μl |

1:0.001 PCR Reaction Composition

| | |
|---|---|
| AmpliTaq Gold Master Mix | 10 μl |
| Aliquot of 1:0.001 OLA Reaction Composition | 1 μl |
| PCR Primer A (0.5 μM) | 1 μl |
| PCR Primer B (0.5 μM) | 1 μl |
| Water | 7 μl |
| total volume | 20 μl |

1:0 PCR Reaction Composition

| | |
|---|---|
| AmpliTaq Gold Master Mix | 10 μl |
| Aliquot of 1:0 OLA Reaction Composition | 1 μl |
| PCR Primer A (0.5 μM) | 1 μl |
| PCR Primer B (0.5 μM) | 1 μl |
| Water | 7 μl |
| total volume | 20 μl |

0:1 PCR Reaction Composition

| | |
|---|---|
| AmpliTaq Gold Master Mix | 10 μl |
| Aliquot of 0:1 OLA Reaction Composition | 1 μl |
| PCR Primer A (0.5 μM) | 1 μl |
| PCR Primer B (0.5 μM) | 1 μl |
| Water | 7 μl |
| total volume | 20 μl |

PCR reaction compositions were heated to 95° C. for 5 minutes, and were then incubated under thermal cycling conditions. The thermal cycling conditions were 95° C. for 5 seconds followed by 60° C. for 30 seconds. Those cycling conditions were repeated 30 times. After the PCR reaction compositions were incubated under thermal cycling conditions, those reaction compositions were maintained at 4° C.

### EXAMPLE 10

Preparation of Unmethylated Genomic DNA

300 ng of unmethylated genomic DNA (1 μl of Coriell genomic DNA) was added to 44 μl of water and 5 μl of M-dilution buffer (Zymo Research) to form a bisulfite pre-reaction composition. The bisulfite pre-reaction composition was incubated at 37° C. for 15 minutes.

A bisulfite stock solution (approximately 3M bisulfite) was prepared by adding 210 μl of the M-dilution buffer (Zymo Research) and 750 μl of water to a vial provided by Zymo Research (Zymo Research), which contained Zymo CT conversion reagent. After adding the M-dilution buffer and the water, the bisulfite stock solution was vortexed periodically over a 10 minute period to dissolve the Zymo CT conversion reagent. After dissolving the Zymo CT conversion reagent, 100 μl of the bisulfite stock solution was removed and added to the bisulfite pre-reaction composition to form a bisulfite reaction composition. The bisulfite reaction composition was incubated for 15 hours at 50° C.

Following the incubation, 300 μl of water was added to the bisulfite reaction composition, and the resulting solution was transferred to the upper chamber of a Microcon 100 filtration device (Millipore), which was centrifuged at 2800 rpm for 18 minutes. The filtrate was removed and 350 μl of water was added to the upper chamber of the filtration device. The filtration device was centrifuged at 2800 rpm for 15 minutes. The filtrate was removed and another 350 μl of water was added to the upper chamber of the filtration device. The filtration device was centrifuged at 2800 rpm for 15 minutes. The filtrate was removed and 350 μl of 0.1 M NaOH was added to the upper chamber of the filtration device. After adding the NaOH, the filtration device was allowed to sit for 5 minutes, and was then centrifuged at 2800 rpm for 15 minutes. The filtrate was removed and 350 μl of water was added to the upper chamber of the filtration device. The filtration device was centrifuged at 2800 rpm for 15 minutes. The filtrate was removed and 50 μl of TE buffer (10 mM Tris Cl; 1 mM EDTA (pH 8.0) (Teknova, T0223)) was added to the upper chamber of the filtration device with gentle mixing. The filtration device was allowed to sit for 5 minutes after which the device was inverted and the TE buffer containing the bisulfite treated DNA was collected.

The bisulfite treated unmethylated genomic DNA was resuspended at a concentration of 10 ng/μl.

Preparation of Methylated Genomic DNA

300 ng of methylated genomic DNA (3.3 μl of CpGenome universal methylated DNA (Serologicals P/N S7821)) was added to 44 μl of water and 5 μl of M-dilution buffer (Zymo Research) to form a bisulfite pre-reaction composition. The bisulfite pre-reaction composition was incubated at 37° C. for 15 minutes.

A bisulfite stock solution (approximately 3M bisulfite) was prepared by adding 210 μl of the M-dilution buffer (Zymo Research) and 750 μl of water to a vial provided by Zymo Research (Zymo Research), which contained Zymo CT conversion reagent. After adding the M-dilution buffer and the water, the bisulfite stock solution was vortexed periodically over a 10 minute period to dissolve the Zymo CT conversion reagent. After dissolving the Zymo CT conversion reagent, 100 μl of the bisulfite stock solution was removed and added to the bisulfite pre-reaction composition to form a bisulfite reaction composition. The bisulfite reaction composition was incubated for 15 hours at 50° C.

Following the incubation, 300 μl of water was added to the bisulfite reaction composition, and the resulting solution was transferred to the upper chamber of a Microcon 100 filtration device (Millipore), which was centrifuged at 2800 rpm for 18 minutes. The filtrate was removed and 350 μl of water was added to the upper chamber of the filtration device. The filtration device was centrifuged at 2800 rpm for 15 minutes. The filtrate was removed and another 350 μl of water was added to the upper chamber of the filtration device. The filtration device was centrifuged at 2800 rpm for 15 minutes. The filtrate was removed and 350 μl of 0.1 M NaOH was added to the upper chamber of the filtration device. After adding the NaOH, the filtration device was allowed to sit for 5 minutes, and was then centrifuged at 2800 rpm for 15 minutes. The filtrate was removed and 350 μl of water was added to the upper chamber of the filtration device. The filtration device was centrifuged at 2800 rpm for 15 minutes. The filtrate was removed and 50 μl of TE buffer (10 mM Tris Cl; 1 mM EDTA (pH 8.0) (Teknova, T0223)) was added to the upper chamber of the filtration device with gentle mixing. The filtration device was allowed to sit for 5 minutes after which the device was inverted and the TE buffer containing the bisulfite treated DNA was collected.

The bisulfite treated methylated genomic DNA was resuspended at a concentration of 10 ng/μl.

OLA-PCR reactions

Six different target DNA compositions were prepared by mixing bisulfite treated unmethylated genomic DNA with bisulfite treated methylated genomic DNA at six different concentrations. The six ratios of bisulfite treated unmethylated genomic DNA to bisulfite treated methylated genomic DNA are shown below:

Unmethylated (UnMe):Methylated (Me)
1:1
1:0.1
1:0.01
1:0.001
1:0
0:1

The following probes were designed for the following OLA reactions:

| | |
|---|---|
| RasSF ms Me P B-OLA: | |
| ACCCGCGCTTACTAAC | (SEQ ID NO.: X) |
| TTACTCAGGACTCATCGTCGC | |
| RasSF bs Me B-OLA: | |
| CTCGTAGACTGCGTACCGATCCCTATA | (SEQ ID NO.: X) |
| ACCCCGCCCG | |
| RasSF Blocking Probe: | |
| CCCCACCCAACCCACAC | (SEQ ID NO.: X) |

The portions of the probes marked by italics are universal primer specific portions.

Six different OLA reaction compositions were prepared as follows:

1:1 OLA Reaction Composition

| | |
|---|---|
| Taq Ligase buffer 10X | 1 μl |
| Taq Ligase (4 U/μl) | 1 μl |
| Target DNA composition (1:1 UnMe:Me) | 1 μl |
| RasSF ms Me P B-OLA (0.05 μM) | 1 μl |
| RasSF bs Me B-OLA (0.05 μM) | 1 μl |
| RasSF Blocking Probe (0.5 μM) | 1 μl |
| Water | 4 μl |
| total volume | 10 μl |

1:0.1 OLA Reaction Composition

| | |
|---|---|
| Taq Ligase buffer 10X | 1 μl |
| Taq Ligase (4 U/μl) | 1 μl |
| Target DNA composition (1:0.1 UnMe:Me) | 1 μl |
| RasSF ms Me P B-OLA (0.05 μM) | 1 μl |
| RasSF bs Me B-OLA (0.05 μM) | 1 μl |
| RasSF Blocking Probe (0.5 μM) | 1 μl |
| Water | 4 μl |
| total volume | 10 μl |

1:0.01 OLA Reaction Composition

| | |
|---|---|
| Taq Ligase buffer 10X | 1 μl |
| Taq Ligase (4 U/μl) | 1 μl |
| Target DNA composition (1:0.01 UnMe:Me) | 1 μl |
| RasSF ms Me P B-OLA (0.05 μM) | 1 μl |
| RasSF bs Me B-OLA (0.05 μM) | 1 μl |
| RasSF Blocking Probe (0.5 μM) | 1 μl |
| Water | 4 μl |
| total volume | 10 μl |

1:0.001 OLA Reaction Composition

| | |
|---|---|
| Taq Ligase buffer 10X | 1 μl |
| Taq Ligase (4 U/μl) | 1 μl |
| Target DNA composition (1:0.001 UnMe:Me) | 1 μl |
| RasSF ms Me P B-OLA (0.05 μM) | 1 μl |
| RasSF bs Me B-OLA (0.05 μM) | 1 μl |
| RasSF Blocking Probe (0.5 μM) | 1 μl |
| Water | 4 μl |
| total volume | 10 μl |

1:0 OLA Reaction Composition

| | |
|---|---|
| Taq Ligase buffer 10X | 1 μl |
| Taq Ligase (4 U/μl) | 1 μl |
| Target DNA composition (1:0 UnMe:Me) | 1 μl |
| RasSF ms Me P B-OLA (0.05 μM) | 1 μl |
| RasSF bs Me B-OLA (0.05 μM) | 1 μl |
| RasSF Blocking Probe (0.5 μM) | 1 μl |
| Water | 4 μl |
| total volume | 10 μl |

0:1 OLA Reaction Composition

| | |
|---|---|
| Taq Ligase buffer 10X | 1 μl |
| Taq Ligase (4 U/μl) | 1 μl |
| Target DNA composition (0:1 UnMe:Me) | 1 μl |
| RasSF ms Me P B-OLA (0.05 μM) | 1 μl |
| RasSF bs Me B-OLA (0.05 μM) | 1 μl |
| RasSF Blocking Probe (0.5 μM) | 1 μl |
| Water | 4 μl |
| total volume | 10 μl |

Reaction compositions were incubated under thermal cycling conditions. The thermal cycling conditions were 95° C. for 5 seconds followed by 60° C. for 1 minute. Those cycling conditions were repeated 90 times.

Following incubation under thermal cycling conditions, a 1 μl aliquot of each OLA reaction composition was removed and was included in a PCR amplification reaction. The primers used in the PCR reaction were: PCR Primer A (5′ CTCGTAGACTGCGTACCGATC (SEQ ID NO.: X)) and PCR Primer B (5′ (FAM)-GCGACGATGAGTCCTGAGTAA (SEQ ID NO.: X)). The PCR reaction compositions were prepared as follows:

1:1 PCR Reaction Composition

| | |
|---|---|
| AmpliTaq Gold Master Mix | 10 μl |
| Aliquot of 1:1 OLA Reaction Composition | 1 μl |
| PCR Primer A (0.5 μM) | 1 μl |
| PCR Primer B (0.5 μM) | 1 μl |
| Water | 7 μl |
| total volume | 20 μl |

1:0.1 PCR Reaction Composition

| | |
|---|---|
| AmpliTaq Gold Master Mix | 10 μl |
| Aliquot of 1:0.1 OLA Reaction Composition | 1 μl |
| PCR Primer A (0.5 μM) | 1 μl |
| PCR Primer B (0.5 μM) | 1 μl |
| Water | 7 μl |
| total volume | 20 μl |

1:0.01 PCR Reaction Composition

| | |
|---|---|
| AmpliTaq Gold Master Mix | 10 μl |
| Aliquot of 1:0.01 OLA Reaction Composition | 1 μl |
| PCR Primer A (0.5 μM) | 1 μl |
| PCR Primer B (0.5 μM) | 1 μl |
| Water | 7 μl |
| total volume | 20 μl |

1:0.001 PCR Reaction Composition

| | |
|---|---|
| AmpliTaq Gold Master Mix | 10 μl |
| Aliquot of 1:0.001 OLA Reaction Composition | 1 μl |
| PCR Primer A (0.5 μM) | 1 μl |
| PCR Primer B (0.5 μM) | 1 μl |
| Water | 7 μl |
| total volume | 20 μl |

1:0 PCR Reaction Composition

| | |
|---|---|
| AmpliTaq Gold Master Mix | 10 μl |
| Aliquot of 1:0 OLA Reaction Composition | 1 μl |
| PCR Primer A (0.5 μM) | 1 μl |
| PCR Primer B (0.5 μM) | 1 μl |
| Water | 7 μl |
| total volume | 20 μl |

0:1 PCR Reaction Composition

| | |
|---|---|
| AmpliTaq Gold Master Mix | 10 μl |
| Aliquot of 0:1 OLA Reaction Composition | 1 μl |
| PCR Primer A (0.5 μM) | 1 μl |
| PCR Primer B (0.5 μM) | 1 μl |
| Water | 7 μl |
| total volume | 20 μl |

PCR reaction compositions were heated to 95° C. for 5 minutes, and were then incubated under thermal cycling conditions. The thermal cycling conditions were 95° C. for 5 seconds followed by 60° C. for 30 seconds. Those cycling conditions were repeated 30 times. After the PCR reaction compositions were incubated under thermal cycling conditions, those reaction compositions were maintained at 4° C.

### EXAMPLE 11

Human gDNA is bisulfite treated using a published protocol (Boyd and Zon, Anal. Biochem. 326: 278-280, 2004; see also U.S. Provisional Patent Application Nos. 60/520,941; and 60/523,056; and U.S. patent application Ser. Nos. 10/926,530; 10/926,528, which published as U.S. Patent Application Publication No. US 2005-0089898 A1; 10/926,534, which published as U.S. Patent Application Publication No. US 2005-0079527 A1; and 10/926,531, which published as U.S. Patent Application Publication No. US 2005-0095623 A1). A tailed first primer pair comprising a tailed first primer and a tailed second primer is synthesized; the sequences of the tailed first primer and tailed second primer are:

(SEQ ID NO.: X)

CAGGAAACAGCTATGACC[CTACACCCAAATTTCCATTA];
and

(SEQ ID NO.: X)

TGTAAAACGACGGCCAGTTAGTTTAATGAGTTTAGGTTTTTT,

respectively. The target-specific portion of the tailed first primer is shown in brackets; the target-specific portion of the tailed second primer is shown in italics; and the respective tails, each comprising a primer-binding site, are shown underlined. In this exemplary tailed primer pair, the primer-binding sites comprise M13 sequences.

The bisulfite-treated gDNA nucleic acid target sequence being interrogated comprises the sequence: TAGTTTAATGAGTTTAGGTTTTTTCGATATGGTTCGGTTGGGTTCGTGTTTCGTTGGT TTTGGGCGTTAGTAAGCGCGGGTCGGGCGGGGTTATAGGGCGGGTTTCGATTTTA GCGTTTTTTTTAGGATTTAGATTGGGCGGCGGGAAGGAGTTGAGGAGAGTCGCG[TAATGGAAATTTGGGTGTAG] (SEQ ID NO.: X), wherein the first region (to which the target-specific portion of the tailed first primer anneals) is shown in brackets, the second region (the complement of which anneals with the target-specific portion of the tailed second primer) is shown in italics, and the potentially methylated target cytosines are shown underlined.

A amplification reaction composition comprising 1 μL AmpliTaq Gold 10× buffer, 0.8 dNTPs (2.5 mM each), 0.8 μL MgCl₂ (25 nM), 0.2 μL AmpliTaq Gold polymerase, 0.5 μL bisulfite treated gDNA (10 ng/μL), 6.2 μL water, 0.25 μL (5 μM) of the tailed first primer (5 μM) and 0.25 μL (5 μM) tailed second primer is formed in a capped MicroAmp® tube (N8010580, Applied Biosystems). The tube is capped with a MicroAmp® Tube Cap (N8010534, Applied Biosystems), placed in a MicroAmp® 96-well tray retainer (P/N 403081), and the tray is placed in a thermocycler. The reaction composition is heated to 95° C. for 11 minutes to activate the polymerase, then cycled thirty-five times between 97° C. for 5 seconds, 60° C. for 2 seconds (typically 5-10° C. higher than the calculated Tm of the respective complementary portions of the tailed primer pair), and 72° C. for 45 seconds, then cooled to 4° C., to generate an amplification product.

To remove unincorporated dNTPs and single-stranded primers, 1 μL Exo SAP-IT® reagent (USB Corporation, Cleveland, Ohio) is added per 10 μL cycled reaction composition. The tray is heated to 37° C. for 30 minutes, 80° C. for 15 minutes, then cooled to 4° C.

The amplification product includes the nucleic acid target sequence shown below with the underlined portion showing the region for which OLA probes are designed. The potentially methylated target cytosines associated with CpG islands are shown in grey boxes:

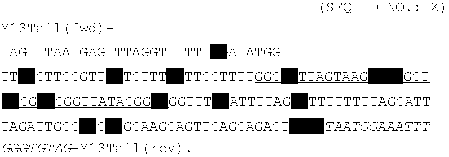

The following OLA probes and blocking probe are designed to detect the methylation state of one or more target nucleotides in a target nucleic acid sequence:

| | |
|---|---|
| RasSF Me P B-OLA | |
| ACCCGCGCTTACTAAC | (SEQ ID NO.: X) |
| RasSF Me FAM B-OLA | |
| (FAM)-CCTATAACCCCGCCCG | (SEQ ID NO.: X) |
| RasSF Blocking Probe | |
| CCCCACCCAACCCACAC | (SEQ ID NO.: X) |

The position of the OLA probes aligned to a target nucleic acid sequence is shown below. The sequence of the RasSF Me FAM B-OLA probe has been italicized to distinguish it from the RasSF Me P B-OLA probe. The gray boxes represent the position of potentially methylated cytosines. In the alignment shown below, none of the potentially methylated cytosines have been converted by bisulfite treatment to thymine, allowing the ligation probe set to hybridize with the target nucleic acid sequence without mismatches.

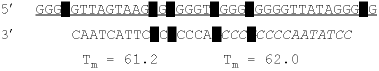

The position of the blocking probe aligned to a target nucleic acid sequence is shown below. The gray boxes represent the position of potentially methylated cytosines. In the sequence alignment shown below, the potentially methylated cytosines are converted to thymine, allowing the blocking probe to hybridize with the target nucleic acid sequence without mismatches.

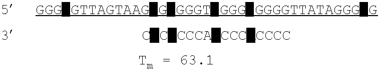

The following reaction composition comprising the RasSF Me P B-OLA and RasSF Me FAM B-OLA primers is prepared:

| | |
|---|---|
| Taq Ligase buffer 10X | 1 μl |
| Taq Ligase (4 U/μl) | 1 μl |
| Amplification Product (approx) | 1 μl |
| FAM-probe (RasSF Me FAM B-OLA 0.5 μM) | 1 μl |
| Phosphate probe (RasSF Me P B-OLA 0.5 μM) | 1 μl |
| Blocking Probe (RasSF Blocking Probe 2-10X of probe conc.) | 1 μl |
| Water | 4 μl |
| total volume | 10 μl |

Reaction compositions are incubated under thermal cycling conditions. The thermal cycling conditions are 97° C. for 5 seconds followed by 60° C. for 1 minute. Those cycling conditions are repeated 60 times.

For fluorescence analysis: Mix 1.2 μl of the product from the OLA reaction with 12 μl of Hi-Di Formamide (Applied Biosystems), heat at 95° C. for 5 minutes and analyze on the ABI Prism 310 (or 3100) Genetic Analyzer.

Although the disclosed teachings have been described with reference to various applications, methods, and compositions, it will be appreciated that various changes and modifications may be made without departing from the teachings herein. The foregoing examples are provided to better illustrate the present teachings and are not intended to limit the scope of the teachings herein. Certain aspects of the present teachings may be further understood in light of the following claims.

1. A method for determining the methylation state of a target nucleotide in at least one target nucleic acid sequence in a sample, comprising:
forming a ligation reaction composition comprising the sample, at least one blocking probe, and a ligation probe set for each target nucleic acid sequence, the ligation probe set comprising (a) a first probe, comprising a first target-specific portion; and (b) a second probe, comprising a second target-specific portion;
subjecting the ligation reaction composition to at least one cycle of ligation, under conditions effective to ligate together first and second probes that are hybridized adjacent to one another on the target nucleic acid sequence if the target nucleotide is methylated to form a ligation product;
wherein the blocking probe hybridizes to a portion of the target nucleic acid sequence comprising the target nucleotide if the target nucleotide is unmethylated; and wherein hybridization of the blocking probe to the portion of the target nucleic acid sequence blocks hybridization of the first probe, the second probe, or both the first probe and the second probe to the portion of the target nucleic acid sequence; and
detecting the presence or absence of the ligation product to determine the methylation state of the target nucleotide.

2. The method of claim 1, wherein the first probe comprises a terminal nucleotide that aligns opposite the target nucleotide if the first probe is hybridized to the target nucleic acid sequence, and the second probe comprises a terminal nucleotide that aligns opposite a nucleotide that is adjacent to the target nucleotide if the second probe is hybridized to the target nucleic acid sequence.

3. The method of claim 1, wherein the detecting comprises separation by a mobility dependent analysis technique.

4. The method of claim 1, wherein the first probe, the second probe, or both the first probe and the second probe comprises at least one mobility modifier.

5. The method of claim 1, wherein the at least one cycle of ligation comprises repeated cycles of ligation.

6. The method of claim 1, wherein the first probe, the second probe, or both the first probe and the second probe is labeled.

7. The method of claim 1, wherein the first probe, the second probe, or both the first probe and the second probe comprises an addressable support-specific portion.

8. The method of claim 1, wherein the first probe comprises a label that has a first detectable signal value when it is ligated to the second probe and has a second detectable signal value when it is not ligated to the second probe.

9. The method of claim 8, wherein the first probe comprises a signal moiety and the second probe comprises a quencher moiety, wherein the quencher moiety changes the detectable signal value from the signal moiety when the first and second probes are ligated together.

10. The method of claim 8, wherein the first probe comprises a signal moiety and the second probe comprises a donor moiety, wherein the donor moiety changes the detectable signal value from the signal moiety when the first and second probes are ligated together.

11. The method of claim 1, wherein at least one of the first probe, the second probe, or both the first probe and the second probe is labeled, and the method further comprises:
after subjecting the ligation reaction composition to at least one cycle of ligation, increasing stringency so that unligated probes are not hybridized to the target nucleic acid sequence;
substantially removing any unhybridized probes from the sample; and
detecting signal from the label.

12. The method of claim 1, wherein the blocking probe comprises at least one modified nucleotide.

13. The method of claim 12, wherein at least one of the at least one nucleotides comprises a modified guanine, and wherein the modified guanine will base pair with unmethylated cytosines but will not base pair with methylated cytosines.

14. The method of claim 1, wherein the target nucleotide is a cytosine.

15. The method of claim 1, wherein the first probe further comprises a 5′ primer-specific portion, wherein the 5′ primer-specific portion comprises a 5′ primer-specific sequence, and wherein the second probe further comprises a 3′ primer-specific portion, wherein the 3′ primer-specific portion comprises a 3′ primer-specific sequence; wherein at least one probe in each ligation probe set further comprises an addressable support-specific portion located between the primer-specific portion and the target-specific portion;
wherein the subjecting the ligation reaction composition to at least one cycle of ligation forms a test composition comprising the ligation product;
wherein after the test composition is formed,
forming an amplification reaction composition comprising:
a portion of the test composition;
at least one primer set, the primer set comprising (i) at least one first primer comprising the 5′ primer-specific sequence, and (ii) at least one second primer comprising a sequence complementary to the 3′ primer-specific sequence; and
a polymerase;
subjecting the amplification reaction composition to at least one cycle of amplification to generate at least one amplification product; and
wherein the detecting the presence or absence of the ligation product to determine the methylation state of the target nucleotide comprises detecting the addressable support specific portion of the at least one amplification product.

16. The method of claim 1, wherein the first probe further comprises a 5′ primer-specific portion, wherein the 5′ primer-specific portion comprises a 5′ primer-specific sequence, and wherein the second probe further comprises a 3′ primer-specific portion, wherein the 3′ primer-specific portion comprises a 3′ primer-specific sequence; wherein at least one probe in each ligation probe set further comprises an addressable support-specific portion located between the primer-specific portion and the target-specific portion; wherein the addressable support-specific portion comprises an addressable support-specific portion sequence,
wherein the subjecting the ligation reaction composition to at least one cycle of ligation forms a test composition comprising the ligation product; and
wherein after the test composition is formed:
forming an amplification reaction composition comprising:
a portion of the test composition;
a polymerase;
a labeled probe, wherein the labeled probe has a first detectable signal value when it is not hybridized to a complementary sequence, and wherein the labeled probe comprises the addressable support-specific portion sequence or comprises a sequence complementary to the addressable support-specific portion sequence; and
at least one primer set, the primer set comprising (i) at least one first primer comprising the 5′ primer-specific sequence, and (ii) at least one second primer comprising a sequence complementary to the 3′ primer-specific sequence;
subjecting the amplification reaction composition to at least one amplification reaction; and
wherein the detecting the presence or absence of the ligation product to determine the methylation state of the target nucleotide comprises detecting a second detectable signal value from the labeled probe either (a) during the amplification reaction, (b) after the amplification reaction, or (c) both during and after the amplification reaction;
wherein a threshold difference between the first detectable signal value and the second detectable signal value of the labeled probe indicates the presence of a methylated target nucleotide; and wherein no threshold difference between the first detectable signal value and the second detectable signal value of the labeled probe indicates the absence of a methylated target nucleotide.

17. The method of claim 16, wherein the labeled probe is a 5′ nuclease probe.

18. A method for determining the methylation state of a target nucleotide in at least one target nucleic acid sequence in a sample, comprising:
forming a ligation reaction composition comprising the sample, at least one blocking probe, and a ligation probe set for each target nucleic acid sequence, the ligation probe set comprising (a) a first probe, comprising a first target-specific portion; and (b) a second probe, comprising a second target-specific portion;
subjecting the ligation reaction composition to at least one cycle of ligation, under conditions effective to ligate together first and second probes that are hybridized adjacent to one another on the target nucleic acid sequence if the target nucleotide is unmethylated to form a ligation product;
wherein the blocking probe hybridizes to a portion of the target nucleic acid sequence comprising the target nucleotide if the target nucleotide is methylated; and wherein hybridization of the blocking probe to the portion of the target nucleic acid sequence blocks hybridization of the first probe, the second probe, or both the first probe and the second probe to the portion of the target nucleic acid sequence; and
detecting the presence or absence of the ligation product to determine the methylation state of the target nucleotide.

19. The method of claim 18, wherein the first probe comprises a terminal nucleotide that aligns opposite the target nucleotide if the first probe is hybridized to the target nucleic acid sequence, and the second probe comprises a terminal nucleotide that aligns opposite a nucleotide that is adjacent to the target nucleotide if the second probe is hybridized to the target nucleic acid sequence.

20. The method of claim 18, wherein the detecting comprises separation by a mobility dependent analysis technique.

21. The method of claim 18, wherein the first probe, the second probe, or both the first probe and the second probe comprises at least one mobility modifier.

22. The method of claim 18, wherein the at least one cycle of ligation comprises repeated cycles of ligation.

23. The method of claim 18, wherein the first probe, the second probe, or both the first probe and the second probe is labeled.

24. The method of claim 18, wherein the first probe, the second probe, or both the first probe and the second probe comprises an addressable support-specific portion.

25. The method of claim 18, wherein the first probe comprises a label that has a first detectable signal value when it is ligated to the second probe and has a second detectable signal value when it is not ligated to the second probe.

26. The method of claim 25, wherein the first probe comprises a signal moiety and the second probe comprises a quencher moiety, wherein the quencher moiety changes the detectable signal value from the signal moiety when the first and second probes are ligated together.

27. The method of claim 25, wherein the first probe comprises a signal moiety and the second probe comprises a donor moiety, wherein the donor moiety changes the detectable signal value from the signal moiety when the first and second probes are ligated together.

28. The method of claim 18, wherein at least one of the first probe, the second probe, or both the first probe and the second probe is labeled, and the method further comprises:
after subjecting the ligation reaction composition to at least one cycle of ligation, increasing stringency so that unligated probes are not hybridized to the target nucleic acid sequence;
substantially removing any unhybridized probes from the sample; and
detecting signal from the label.

29. The method of claim 18, wherein the at least one blocking probe comprises at least one modified nucleotide.

30. The method of claim 18, wherein the first probe further comprises a 5′ primer-specific portion, wherein the 5′ primer-specific portion comprises a 5′ primer-specific sequence, and wherein the second probe further comprises a 3′ primer-specific portion, wherein the 3′ primer-specific portion comprises a 3′ primer-specific sequence; wherein at least one probe in each ligation probe set further comprises an addressable support-specific portion located between the primer-specific portion and the target-specific portion;
wherein the subjecting the ligation reaction composition to at least one cycle of ligation forms a test composition comprising the ligation product;
wherein after the test composition is formed,
forming an amplification reaction composition comprising:
a portion of the test composition;
at least one primer set, the primer set comprising (i) at least one first primer comprising the 5′ primer-specific sequence, and (ii) at least one second primer comprising a sequence complementary to the 3′ primer-specific sequence; and
a polymerase;
subjecting the amplification reaction composition to at least one cycle of amplification to generate at least one amplification product; and
wherein the detecting the presence or absence of the ligation product to determine the methylation state of the target nucleotide comprises detecting the addressable support specific portion of the at least one amplification product.

31. The method of claim 18, wherein the first probe further comprises a 5′ primer-specific portion, wherein the 5′ primer-specific portion comprises a 5′ primer-specific sequence, and wherein the second probe further comprises a 3′ primer-specific portion, wherein the 3′ primer-specific portion comprises a 3′ primer-specific sequence; wherein at least one probe in each ligation probe set further comprises an addressable support-specific portion located between the primer-specific portion and the target-specific portion; wherein the addressable support-specific portion comprises an addressable support-specific portion sequence,
wherein the subjecting the ligation reaction composition to at least one cycle of ligation forms a test composition comprising the ligation product; and
wherein after the test composition is formed:
forming an amplification reaction composition comprising:
a portion of the test composition;
a polymerase;
a labeled probe, wherein the labeled probe has a first detectable signal value when it is not hybridized to a complementary sequence, and wherein the labeled probe comprises the addressable support-specific portion sequence or comprises a sequence complementary to the addressable support-specific portion sequence; and
at least one primer set, the primer set comprising (i) at least one first primer comprising the 5′ primer-specific sequence, and (ii) at least one second primer comprising a sequence complementary to the 3′ primer-specific sequence;
subjecting the amplification reaction composition to at least one amplification reaction; and
wherein the detecting the presence or absence of the ligation product to determine the methylation state of the target nucleotide comprises detecting a second detectable signal value from the labeled probe either (a) during the amplification reaction, (b) after the amplification reaction, or (c) both during and after the amplification reaction;
wherein a threshold difference between the first detectable signal value and the second detectable signal value of the labeled probe indicates the presence of an unmethylated target nucleotide; and wherein no threshold difference between the first detectable signal value and the second detectable signal value of the labeled probe indicates the absence of an unmethylated target nucleotide.

32. The method of claim 31, wherein the labeled probe is a 5′ nuclease probe.

33. A kit for determining the methylation state of a target nucleotide in at least one target nucleic acid sequence in a sample comprising:
at least one blocking probe; and
a ligation probe set for each target nucleic acid sequence, the ligation probe set comprising:
(a) a first probe, comprising a first target-specific portion, and
(b) a second probe, comprising a second target-specific portion;
wherein the first probe and the second probe in each ligation probe set are suitable for ligation together when hybridized adjacent to one another on a complementary target nucleic acid sequence; and
wherein the blocking probe is capable of hybridizing to a portion of the target nucleic acid sequence comprising the target nucleotide if the target nucleotide is unmethylated, and wherein hybridization of the blocking probe to the portion of the target nucleic acid sequence blocks hybridization of the first probe, the second probe, or both the first probe and the second probe to the portion of the target nucleic acid sequence.

34.-44. (canceled)

45. A method for determining the methylation state of a target nucleotide in at least one target nucleic acid sequence in a sample, comprising:
forming a test composition by incubating the at least one target nucleic acid sequence with a modifying agent that modifies an unmethylated target nucleotide to a modified target nucleotide, but does not modify a methylated target nucleotide to the modified target nucleotide, to obtain at least one test target nucleic acid sequence;
forming a ligation reaction composition comprising at least a portion of the test composition, at least one blocking probe, and a ligation probe set for each target nucleic acid sequence, the ligation probe set comprising (a) a first probe, comprising a first target-specific portion; and (b) a second probe, comprising a second target-specific portion, wherein the first probe and the second probe in each ligation probe set are suitable for ligation together when hybridized adjacent to one another on a complementary test target nucleic acid sequence, and wherein at least one of the first probe and the second probe of each ligation probe set comprises a test nucleotide that aligns opposite the target nucleotide if the probe is hybridized to the test target nucleic acid sequence, wherein the test nucleotide is complementary to the target nucleotide;
wherein the blocking probe hybridizes to a portion of the test target nucleic acid sequence comprising the modified target nucleotide if the target nucleotide has been modified to the modified target nucleotide; and wherein hybridization of the blocking probe to the portion of the test target nucleic acid sequence blocks hybridization of the first probe, the second probe, or both the first probe and the second probe to the portion of the test target nucleic acid sequence; and
subjecting the ligation reaction composition to at least one cycle of ligation, wherein adjacently hybridizing probes are ligated together to form a ligation product; and
detecting the presence or absence of the ligation product to determine the methylation state of the target nucleotide.

46. The method of claim 45, wherein the test nucleotide is a terminal nucleotide of the first probe, and the second probe comprises a terminal nucleotide that aligns opposite the nucleotide adjacent to the target nucleotide or the modified target nucleotide if the second probe is hybridized to the test target nucleic acid sequence.

47. The method of claim 45, wherein the detecting comprises separation by a mobility dependent analysis technique.

48. The method of claim 45, wherein the first probe, the second probe, or the first probe and the second probe comprise at least one mobility modifier.

49. The method of claim 45, wherein the at least one cycle of ligation comprises repeated cycles of ligation.

50. The method of claim 45, wherein the first probe, the second probe, or the first probe and the second probe are labeled.

51. The method of claim 45, wherein the first probe, the second probe, or the first probe and the second probe comprise an addressable support-specific portion.

52. The method of claim 45, wherein the first probe comprises a label that has a first detectable signal value when it is ligated to the second probe and has a second detectable signal value when it is not ligated to the second probe.

53. The method of claim 52, wherein the first probe comprises a signal moiety and the second probe comprises a quencher moiety, wherein the quencher moiety changes the detectable signal value from the signal moiety when the first and second probes are ligated together.

54. The method of claim 52, wherein the first probe comprises a signal moiety and the second probe comprises a donor moiety, wherein the donor moiety changes the detectable signal value from the signal moiety when the first and second probes are ligated together.

55. The method of claim 45, wherein at least one of the first probe, the second probe, or both the first probe and the second probe is labeled, and the method further comprises:
after subjecting the ligation reaction composition to at least one cycle of ligation, increasing stringency so that unligated probes are not hybridized to the target nucleic acid sequence;
substantially removing any unhybridized probes from the sample; and
detecting signal from the label.

56. The method of claim 45, wherein the modifying agent is bisulfite.

57. The method of claim 45, wherein the modifying agent that modifies an unmethylated target nucleotide to a modified target nucleotide converts the unmethylated target nucleotide to a converted nucleotide.

58. The method of claim 45, wherein the target nucleotide is cytosine.

59. The method of claim 57, wherein the target nucleotide is cytosine and the converted nucleotide is uracil.

60. The method of claim 45, wherein the first probe further comprises a 5′ primer-specific portion, wherein the 5′ primer-specific portion comprises a 5′ primer-specific sequence, and wherein the second probe further comprises a 3′ primer-specific portion, wherein the 3′ primer-specific portion comprises a 3′ primer-specific sequence; wherein at least one probe in each probe set further comprises an addressable support-specific portion located between the primer-specific portion and the target-specific portion;
wherein the subjecting the ligation reaction composition to at least one cycle of ligation forms a test composition comprising the ligation product;
wherein after the test composition is formed,
forming an amplification reaction composition comprising:
a portion of the test composition;
at least one primer set, the primer set comprising (i) at least one first primer comprising the 5′ primer-specific sequence, and (ii) at least one second primer comprising a sequence complementary to the 3′ primer-specific sequence; and
a polymerase;
subjecting the amplification reaction composition to at least one cycle of amplification to generate at least one amplification product; and
wherein the detecting the presence or absence of the ligation product to determine the methylation state of the target nucleotide comprises detecting the addressable support specific portion of the at least one amplification product.

61. The method of claim 45, wherein the first probe further comprises a 5′ primer-specific portion, wherein the 5′ primer-specific portion comprises a 5′ primer-specific sequence, and wherein the second probe further comprises a 3′ primer-specific portion, wherein the 3′ primer-specific portion comprises a 3′ primer-specific sequence; wherein at least one probe in each ligation probe set further comprises an addressable support-specific portion located between the primer-specific portion and the target-specific portion; wherein the addressable support-specific portion comprises an addressable support-specific portion sequence,
wherein the subjecting the ligation reaction composition to at least one cycle of ligation forms a test composition comprising the ligation product; and
wherein after the test composition is formed:
forming an amplification reaction composition comprising:
a portion of the test composition;
a polymerase;
a labeled probe, wherein the labeled probe has a first detectable signal value when it is not hybridized to a complementary sequence, and wherein the labeled probe comprises the addressable support-specific portion sequence or comprises a sequence complementary to the addressable support-specific portion sequence; and at least one primer set, the primer set comprising (i) at least one first primer comprising the 5′ primer-specific sequence, and (ii) at least one second primer comprising a sequence complementary to the 3′ primer-specific sequence;
subjecting the amplification reaction composition to at least one amplification reaction; and
wherein the detecting the presence or absence of the ligation product to determine the methylation state of the target nucleotide comprises detecting a second detectable signal value from the labeled probe either (a) during the amplification reaction, (b) after the amplification reaction, or (c) both during and after the amplification reaction;
wherein a threshold difference between the first detectable signal value and the second detectable signal value of the labeled probe indicates the presence of a methylated target nucleotide; and wherein no threshold difference between the first detectable signal value and the second detectable signal value of the labeled probe indicates the absence of a methylated target nucleotide.

62. The method of claim 61, wherein the labeled probe is a 5′ nuclease probe.

63. A method for determining the methylation state of a target nucleotide in at least one target nucleic acid sequence in a sample, comprising:
forming a test composition by incubating the at least one target nucleic acid sequence with a modifying agent that modifies an unmethylated target nucleotide to a modified target nucleotide, but does not modify a methylated target nucleotide to the modified target nucleotide, to obtain at least one test target nucleic acid sequence;
forming a ligation reaction composition comprising at least a portion of the test composition, at least one blocking probe, and a ligation probe set for each target nucleic acid sequence, the ligation probe set comprising (a) a first probe, comprising a first target-specific portion; and (b) a second probe, comprising a second target-specific portion, wherein the first probe and the second probe in each ligation probe set are suitable for ligation together when hybridized adjacent to one another on a complementary test target nucleic acid sequence, and wherein at least one of the first probe and the second probe of each ligation probe set comprises a test nucleotide that aligns opposite the modified target nucleotide if the probe is hybridized to the test target nucleic acid sequence, wherein the test nucleotide is complementary to the modified target nucleotide;
wherein the blocking probe hybridizes to a portion of the test target nucleic acid sequence comprising the target nucleotide if the target nucleotide has not been modified to the modified target nucleotide; and wherein hybridization of the blocking probe to the portion of the test target nucleic acid sequence blocks hybridization of the first probe, the second probe, or both the first probe and the second probe to the portion of the test target nucleic acid sequence; and
subjecting the ligation reaction composition to at least one cycle of ligation, wherein adjacently hybridizing probes are ligated together to form a ligation product; and
detecting the presence or absence of the ligation product to determine the methylation state of the target nucleotide.

64. The method of claim 63, wherein the test nucleotide is a terminal nucleotide of the first probe, and the second probe comprises a terminal nucleotide that aligns opposite the nucleotide adjacent to the target nucleotide or the modified target nucleotide if the second probe is hybridized to the test target nucleic acid sequence.

65. The method of claim 63, wherein the detecting comprises separation by a mobility dependent analysis technique.

66. The method of claim 63, wherein the first probe, the second probe, or the first probe and the second probe comprise at least one mobility modifier.

67. The method of claim 63, wherein the at least one cycle of ligation comprises repeated cycles of ligation.

68. The method of claim 63, wherein the first probe, the second probe, or the first probe and the second probe are labeled.

69. The method of claim 63, wherein the first probe, the second probe, or the first probe and the second probe comprise an addressable support-specific portion.

70. The method of claim 63, wherein the first probe comprises a label that has a first detectable signal value when it is ligated to the second probe and has a second detectable signal value when it is not ligated to the second probe.

71. The method of claim 70, wherein the first probe comprises a signal moiety and the second probe comprises a quencher moiety, wherein the quencher moiety changes the detectable signal value from the signal moiety when the first and second probes are ligated together.

72. The method of claim 70, wherein the first probe comprises a signal moiety and the second probe comprises a donor moiety, wherein the donor moiety changes the detectable signal value from the signal moiety when the first and second probes are ligated together.

73. The method of claim 63, wherein at least one of the first probe, the second probe, or both the first probe and the second probe is labeled, and the method further comprises:
after subjecting the ligation reaction composition to at least one cycle of ligation, increasing stringency so that unligated probes are not hybridized to the target nucleic acid sequence;
substantially removing any unhybridized probes from the sample; and
detecting signal from the label.

74. The method of claim 63, wherein the modifying agent is bisulfite.

75. The method of claim 63, wherein the modifying agent that modifies an unmethylated target nucleotide to a modified target nucleotide converts the unmethylated target nucleotide to a converted nucleotide.

76. The method of claim 63, wherein the target nucleotide is cytosine.

77. The method of claim 75, wherein the target nucleotide is cytosine and the converted nucleotide is uracil.

78. The method of claim 63, wherein the first probe further comprises a 5′ primer-specific portion, wherein the 5′ primer-specific portion comprises a 5′ primer-specific sequence, and wherein the second probe further comprises a 3′ primer-specific portion, wherein the 3′ primer-specific portion comprises a 3′ primer-specific sequence; wherein at least one probe in each ligation probe set further comprises an addressable support-specific portion located between the primer-specific portion and the target-specific portion;
wherein the subjecting the ligation reaction composition to at least one cycle of ligation forms a test composition comprising the ligation product;
wherein after the test composition is formed,
forming an amplification reaction composition comprising:
a portion of the test composition;
at least one primer set, the primer set comprising (i) at least one first primer comprising the 5′ primer-specific sequence, and (ii) at least one second primer comprising a sequence complementary to the 3′ primer-specific sequence; and
a polymerase;
subjecting the amplification reaction composition to at least one cycle of amplification to generate at least one amplification product; and
wherein the detecting the presence or absence of the ligation product to determine the methylation state of the target nucleotide comprises detecting the addressable support specific portion of the at least one amplification product.

79. The method of claim 63, wherein the first probe further comprises a 5′ primer-specific portion, wherein the 5′ primer-specific portion comprises a 5′ primer-specific sequence, and wherein the second probe further comprises a 3′ primer-specific portion, wherein the 3′ primer-specific portion comprises a 3′ primer-specific sequence; wherein at least one probe in each ligation probe set further comprises an addressable support-specific portion located between the primer-specific portion and the target-specific portion; wherein the addressable support-specific portion comprises an addressable support-specific portion sequence,
wherein the subjecting the ligation reaction composition to at least one cycle of ligation forms a test composition comprising the ligation product; and
wherein after the test composition is formed:
forming an amplification reaction composition comprising:
a portion of the test composition;
a polymerase;
a labeled probe, wherein the labeled probe has a first detectable signal value when it is not hybridized to a complementary sequence, and wherein the labeled probe comprises the addressable support-specific portion sequence or comprises a sequence complementary to the addressable support-specific portion sequence; and
at least one primer set, the primer set comprising (i) at least one first primer comprising the 5′ primer-specific sequence, and (ii) at least one second primer comprising a sequence complementary to the 3′ primer-specific sequence;
subjecting the amplification reaction composition to at least one amplification reaction; and
wherein the detecting the presence or absence of the ligation product to determine the methylation state of the target nucleotide comprises detecting a second detectable signal value from the labeled probe either (a) during the amplification reaction, (b) after the amplification reaction, or (c) both during and after the amplification reaction;
wherein a threshold difference between the first detectable signal value and the second detectable signal value of the labeled probe indicates the presence of an unmethylated target nucleotide; and wherein no threshold difference between the first detectable signal value and the second detectable signal value of the labeled probe indicates the absence of an unmethylated target nucleotide.

80. The method of claim 79, wherein the labeled probe is a 5′ nuclease probe.

81.-132. (canceled)

133. A method for detecting a first nucleotide at a test position in at least one first target nucleic acid sequence in a sample, wherein the sample comprises at least one second target nucleic acid sequence comprising a second different nucleotide at the test position, comprising:
forming a ligation reaction composition comprising:
the sample;
at least one blocking probe, comprising at least one modification that either:
(a) increases the affinity of the blocking probe for a nucleic acid sequence that is exactly complementary to the blocking probe sequence without any mismatches,
(b) decreases the affinity of the blocking probe for a nucleic acid sequence that differs by at least one nucleotide from a sequence that is complementary to the blocking probe without any mismatches, or
(c) both increases the affinity of the blocking probe for a nucleic acid sequence that is complementary to the blocking probe sequence without any mismatches and decreases the affinity of the blocking probe for a nucleic acid sequence that differs by at least one nucleotide from a sequence that is complementary to the blocking probe without any mismatches; and
a ligation probe set for the at least one first target nucleic acid sequence; the ligation probe set comprising:
(a) a first probe, comprising a first target-specific portion; and
(b) a second probe, comprising a second target-specific portion;
subjecting the ligation reaction composition to at least one cycle of ligation, under conditions effective to ligate together first and second probes that are hybridized adjacent to one another on the first target nucleic acid sequence if the first nucleotide is present at the test position to form a ligation product;
wherein the blocking probe hybridizes to a portion of the second target nucleic acid sequence comprising the second different nucleotide at the test position; and wherein hybridization of the blocking probe to the portion of the second target nucleic acid sequence blocks hybridization of the first probe, the second probe, or the first probe and the second probe to the portion of the second target nucleic acid sequence; and
detecting the presence or absence of the ligation product to detect the first nucleotide at the test position in the at least one first target nucleic acid sequence.

134.-143. (canceled)

144. The method of claim 133, wherein the blocking probe is attached to at least one minor groove binder group.

145.-150. (canceled)

151. (canceled)


SEQUENCE LISTING (69 sequences)

SEQ ID NO: 1 - DNA, 17 bp, Artificial Sequence
  Description of Artificial Sequence Synthetic oligonucleotide
cgacgctaaccaaaccc

SEQ ID NO: 2 - DNA, 15 bp, Artificial Sequence
  Description of Artificial Sequence Synthetic oligonucleotide
ctaatccccgcgccg

SEQ ID NO: 3 - DNA, 20 bp, Artificial Sequence
  Description of Artificial Sequence Synthetic oligonucleotide
cccacaccacaacactaacc

SEQ ID NO: 4 - DNA, 17 bp, Artificial Sequence
  Description of Artificial Sequence Synthetic oligonucleotide
caacactaaccaaaccc

SEQ ID NO: 5 - DNA, 15 bp, Artificial Sequence
  Description of Artificial Sequence Synthetic oligonucleotide
ctaatccccacacca

SEQ ID NO: 6 - DNA, 30 bp, Artificial Sequence
  Description of Artificial Sequence Synthetic oligonucleotide
  This sequence may encompass 1 to 10 cag repeating units
cagcagcagcagcagcagcagcagcagcag

SEQ ID NO: 7 - DNA, 25 bp, Artificial Sequence
  Description of Artificial Sequence Synthetic oligonucleotide
taggttttttaggaaggagagagtg

SEQ ID NO: 8 - DNA, 22 bp, Artificial Sequence
  Description of Artificial Sequence Synthetic oligonucleotide
ctaaaaccccaactacctaaat

SEQ ID NO: 9 - DNA, 27 bp, Artificial Sequence
  Description of Artificial Sequence Synthetic oligonucleotide
tttagtaattttaggttagagggttat

SEQ ID NO: 10 - DNA, 22 bp, Artificial Sequence
  Description of Artificial Sequence Synthetic oligonucleotide
taactacaaccaaataaacccc

SEQ ID NO: 11 - DNA, 13 bp, Artificial Sequence
  Description of Artificial Sequence Synthetic oligonucleotide
acccgcccacccg

SEQ ID NO: 12 - DNA, 19 bp, Artificial Sequence
  Description of Artificial Sequence Synthetic oligonucleotide
ccccaaaacgaaactaacg

SEQ ID NO: 13 - DNA, 20 bp, Artificial Sequence
  Description of Artificial Sequence Synthetic oligonucleotide
acaaaactaacaacccaccc

SEQ ID NO: 14 - DNA, 13 bp, Artificial Sequence
  Description of Artificial Sequence Synthetic oligonucleotide
acccacccaccca

SEQ ID NO: 15 - DNA, 19 bp, Artificial Sequence
  Description of Artificial Sequence Synthetic oligonucleotide
ccccaaaacaaaactaaca

SEQ ID NO: 16 - DNA, 25 bp, Artificial Sequence
  Description of Artificial Sequence Synthetic oligonucleotide
ttagagtagagggtgaaggtttttt

SEQ ID NO: 17 - DNA, 26 bp, Artificial Sequence
  Description of Artificial Sequence Synthetic oligonucleotide
aacaaactaaataaccaatccaaaac

SEQ ID NO: 18 - DNA, 20 bp, Artificial Sequence
  Description of Artificial Sequence Synthetic oligonucleotide
cgacgtaaactcgctaaaac

SEQ ID NO: 19 - DNA, 25 bp, Artificial Sequence
  Description of Artificial Sequence Synthetic oligonucleotide
caaataaattaaaactacgactacg

SEQ ID NO: 20 - DNA, 26 bp, Artificial Sequence
  Description of Artificial Sequence Synthetic oligonucleotide
actacaactacacaacataaactcac

SEQ ID NO: 21 - DNA, 20 bp, Artificial Sequence
  Description of Artificial Sequence Synthetic oligonucleotide
caacataaactcactaaaac

SEQ ID NO: 22 - DNA, 25 bp, Artificial Sequence
  Description of Artificial Sequence Synthetic oligonucleotide
caaataaattaaaactacaactaca

SEQ ID NO: 23 - DNA, 24 bp, Artificial Sequence
  Description of Artificial Sequence Synthetic oligonucleotide
tagtttaatgagtttaggtttttt

SEQ ID NO: 24 - DNA, 20 bp, Artificial Sequence
  Description of Artificial Sequence Synthetic oligonucleotide
ctacacccaaatttccatta

SEQ ID NO: 25 - DNA, 16 bp, Artificial Sequence
  Description of Artificial Sequence Synthetic oligonucleotide
acccgcgcttactaac

SEQ ID NO: 26 - DNA, 16 bp, Artificial Sequence
  Description of Artificial Sequence Synthetic oligonucleotide
cctataaccccgcccg

SEQ ID NO: 27 - DNA, 17 bp, Artificial Sequence
  Description of Artificial Sequence Synthetic oligonucleotide
ccccacccaacccacac

SEQ ID NO: 28 - DNA, 16 bp, Artificial Sequence
  Description of Artificial Sequence Synthetic oligonucleotide
acccacacttactaac

SEQ ID NO: 29 - DNA, 16 bp, Artificial Sequence
  Description of Artificial Sequence Synthetic oligonucleotide
cctataaccccaccca

SEQ ID NO: 30 - DNA, 42 bp, Artificial Sequence
  Description of Artificial Sequence Synthetic oligonucleotide
ccgaaaacctactaaaaaaccttactcaggactcatcgtcgc

SEQ ID NO: 31 - DNA, 37 bp, Artificial Sequence
  Description of Artificial Sequence Synthetic oligonucleotide
ctcgtagactgcgtaccgatccttccgctatcccgcg

SEQ ID NO: 32 - DNA, 22 bp, Artificial Sequence
  Description of Artificial Sequence Synthetic oligonucleotide
actatcccacaccaaaaaccta

SEQ ID NO: 33 - DNA, 21 bp, Artificial Sequence
  Description of Artificial Sequence Synthetic oligonucleotide
ctcgtagactgcgtaccgatc

SEQ ID NO: 34 - DNA, 21 bp, Artificial Sequence
  Description of Artificial Sequence Synthetic oligonucleotide
gcgacgatgagtcctgagtaa

SEQ ID NO: 35 - DNA, 41 bp, Artificial Sequence
  Description of Artificial Sequence Synthetic oligonucleotide
cgaccgtaaccaaccaatcattactcaggactcatcgtcgc

SEQ ID NO: 36 - DNA, 35 bp, Artificial Sequence
  Description of Artificial Sequence Synthetic oligonucleotide
ctcgtagactgcgtaccgatcccgaccccgaaccg

SEQ ID NO: 37 - DNA, 20 bp, Artificial Sequence
  Description of Artificial Sequence Synthetic oligonucleotide
caaccccaaaccacaaccat

SEQ ID NO: 38 - DNA, 34 bp, Artificial Sequence
  Description of Artificial Sequence Synthetic oligonucleotide
acccgcccacccgttactcaggactcatcgtcgc

SEQ ID NO: 39 - DNA, 40 bp, Artificial Sequence
  Description of Artificial Sequence Synthetic oligonucleotide
ctcgtagactgcgtaccgatcccccaaaacgaaactaacg

SEQ ID NO: 40 - DNA, 41 bp, Artificial Sequence
  Description of Artificial Sequence Synthetic oligonucleotide
cgacgtaaactcgctaaaacttactcaggactcatcgtcgc

SEQ ID NO: 41 - DNA, 46 bp, Artificial Sequence
  Description of Artificial Sequence Synthetic oligonucleotide
ctcgtagactgcgtaccgatccaaataaattaaaactacgactacg

SEQ ID NO: 42 - DNA, 38 bp, Artificial Sequence
  Description of Artificial Sequence Synthetic oligonucleotide
cgacgctaaccaaacccttactcaggactcatcgtcgc

SEQ ID NO: 43 - DNA, 36 bp, Artificial Sequence
  Description of Artificial Sequence Synthetic oligonucleotide
ctcgtagactgcgtaccgatcctaatccccgcgccg

SEQ ID NO: 44 - DNA, 37 bp, Artificial Sequence
  Description of Artificial Sequence Synthetic oligonucleotide
acccgcgcttactaacttactcaggactcatcgtcgc

SEQ ID NO: 45 - DNA, 37 bp, Artificial Sequence
  Description of Artificial Sequence Synthetic oligonucleotide
ctcgtagactgcgtaccgatccctataaccccgcccg

SEQ ID NO: 46 - DNA, 38 bp, Artificial Sequence
  Description of Artificial Sequence Synthetic oligonucleotide
caggaaacagctatgaccctacacccaaatttccatta

SEQ ID NO: 47 - DNA, 42 bp, Artificial Sequence
  Description of Artificial Sequence Synthetic oligonucleotide
tgtaaaacgacggccagttagtttaatgagtttaggtttttt

SEQ ID NO: 48 - DNA, 187 bp, Artificial Sequence
  Description of Artificial Sequence Synthetic oligonucleotide
tagtttaatgagtttaggttttttcgatatggttcggttgggttcgtgtttcgttggttttgggcgttagtaagcgcgggtcgggcggggttatagggcgggtttcgattttagcgttttttttaggatttagattgggcggcgggaaggagttgaggagagtcgcgtaatggaaatttgggtgtag

SEQ ID NO: 49 - DNA, 187 bp, Artificial Sequence; identical to SEQ ID NO: 48
  Description of Artificial Sequence Synthetic oligonucleotide

SEQ ID NO: 50 - DNA, 39 bp, Artificial Sequence
  Description of Artificial Sequence Synthetic oligonucleotide
gggcgttagtaagcgcgggtcgggcggggttatagggcg

SEQ ID NO: 51 - DNA, 39 bp, Artificial Sequence
  Description of Artificial Sequence Synthetic oligonucleotide
gggcgttagtaagtgtgggttgggtggggttatagggcg

SEQ ID NO: 52 - DNA, 28 bp, Artificial Sequence
  Description of Artificial Sequence Synthetic oligonucleotide
ggcgctgcccaacgcaccgaatagttac

SEQ ID NO: 53 - DNA, 28 bp, Artificial Sequence
  Description of Artificial Sequence Synthetic oligonucleotide
  modified_base (3): methylated cytosine
  modified_base (13): methylated cytosine
  modified_base (18): methylated cytosine
  modified_base (28): methylated cytosine
ggcgctgcccaacgcaccgaatagttac

SEQ ID NO: 54 - DNA, 12 bp, Artificial Sequence
  Description of Artificial Sequence Synthetic oligonucleotide
ttgggcagcgcc

SEQ ID NO: 55 - DNA, 16 bp, Artificial Sequence
  Description of Artificial Sequence Synthetic oligonucleotide
gtaactattcggtgcg

SEQ ID NO: 56 - DNA, 28 bp, Artificial Sequence
  Description of Artificial Sequence Synthetic oligonucleotide
  modified_base (1): modified guanine
  modified_base (11): modified guanine
  modified_base (16): modified guanine
  modified_base (26): modified guanine
gtaactattcggtgcgttgggcagcgcc

SEQ ID NO: 57 - DNA, 28 bp, Artificial Sequence
  Description of Artificial Sequence Synthetic oligonucleotide
gtaactattcggtgcgttgggcagcgcc

SEQ ID NO: 58 - DNA, 28 bp, Artificial Sequence
  Description of Artificial Sequence Synthetic oligonucleotide
ggcgttgtttaacgtatcgaatagttac

SEQ ID NO: 59 - DNA, 28 bp, Artificial Sequence
  Description of Artificial Sequence Synthetic oligonucleotide
ggtgttgtttaatgtattgaatagttat

SEQ ID NO: 60 - DNA, 12 bp, Artificial Sequence
  Description of Artificial Sequence Synthetic oligonucleotide
ttaaacaacgcc

SEQ ID NO: 61 - DNA, 16 bp, Artificial Sequence
  Description of Artificial Sequence Synthetic oligonucleotide
gtaactattcgatacg

SEQ ID NO: 62 - DNA, 28 bp, Artificial Sequence
  Description of Artificial Sequence Synthetic oligonucleotide
ataactattcaatacattaaacaacacc

SEQ ID NO: 63 - DNA, 28 bp, Artificial Sequence
  Description of Artificial Sequence Synthetic oligonucleotide
gtaactattcgatacgttaaacaacgcc

SEQ ID NO: 64 - DNA, 27 bp, Artificial Sequence
  Description of Artificial Sequence Synthetic oligonucleotide
tcctgagaaatcccaactgatagtatt

SEQ ID NO: 65 - DNA, 27 bp, Artificial Sequence
  Description of Artificial Sequence Synthetic oligonucleotide
tcctgagaaatcctaactgatagtatt

SEQ ID NO: 66 - DNA, 13 bp, Artificial Sequence
  Description of Artificial Sequence Synthetic oligonucleotide
ggatttctcagga

SEQ ID NO: 67 - DNA, 14 bp, Artificial Sequence
  Description of Artificial Sequence Synthetic oligonucleotide
aatactatcagtta

SEQ ID NO: 68 - DNA, 14 bp, Artificial Sequence
  Description of Artificial Sequence Synthetic oligonucleotide
atcagttagggatt

SEQ ID NO: 69 - DNA, 27 bp, Artificial Sequence
  Description of Artificial Sequence Synthetic oligonucleotide
aatactatcagttaggatttctcagga